(19) United States
(12) Patent Application Publication
(10) Pub. No.: US 2005/0020587 A1
(43) Pub. Date: Jan. 27, 2005

(54) Nicotinamide derivatives useful as PDE4 inhibitors

(75) Inventors: Simon Bailey, San Diego, CA (US); Christopher Gordon Barber, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/896,355

(22) Filed: Jul. 20, 2004

Related U.S. Application Data
(60) Provisional application No. 60/497,069, filed on Aug. 22, 2003.

(30) Foreign Application Priority Data
Jul. 25, 2003 (GB) 0317472.9

(51) Int. Cl. A61K 31/541; A61K 31/5377; A61K 31/4436; A61K 31/496; C 07D 4 9/14

(52) U.S. Cl. 514/227.5; 514/232.5; 514/253.01; 514/342; 544/60; 544/124; 544/360; 546/281.1

(57) ABSTRACT

This invention relates to nicotinamide derivatives of general formula (I): 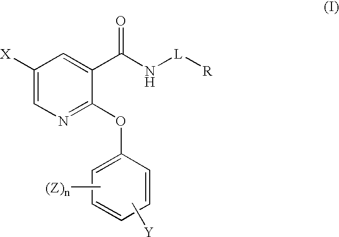 in which X, Y, n, Z, L and R have the meanings defined herein, and to processes for the preparation of, intermediates used in the preparation of, compositions containing and the uses of such derivatives.

This invention relates to nicotinamide derivatives of general formula: 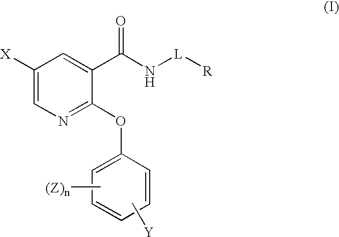
in which X, Z, Y, L, R and n have the meanings indicated below, and to processes for the preparation of, intermediates used in the preparation of, compositions containing and the uses of such derivatives.

The 3′,5′-cyclic nucleotide phosphodiesterases (PDEs) comprise a large class of enzymes divided into at least eleven different families which are structurally, biochemically and pharmacologically distinct from one another. The enzymes within each family are commonly referred to as isoenzymes, or isozymes. A total of more than fifteen gene products is included within this class, and further diversity results from differential splicing and post-translational processing of those gene products. The present invention is primarily concerned with the four gene products of the fourth family of PDEs, i.e., PDE4A, PDE4B, PDE4C, and PDE4D. These enzymes are collectively referred to as being isoforms or subtypes of the PDE4 isozyme family.

The PDE4s are characterized by selective, high affinity hydrolytic degradation of the second messenger cyclic nucleotide, adenosine 3′,5′-cyclic monophosphate (cAMP), and by sensitivity to inhibition by rolipram. A number of selective inhibitors of the PDE4s have been discovered in recent years, and beneficial pharmacological effects resulting from that inhibition have been shown in a variety of disease models (see, e.g., Torphy et al., Environ. Health Perspect. ,1994, 102 Suppl. 10, p. 79-84; Duplantier et al., J. Med. Chem., 1996, 39, p. 120-125 ; Schneider et al., Pharmacol. Biochem. Behav., 1995, 50, p. 211-217; Banner and Page, Br. J. Pharmacol., 1995, 114, p. 93-98 ; Barnette et al., J. Pharmacol. Exp. Ther., 1995, 273, p. 674-679; Wright et al., Can. J. Physiol. Pharmacol., 1997, 75, p. 1001-1008 ; Manabe et al., Eur. J. Pharmacol., 1997, 332, p. 97-107 and Ukita et al., J. Med. Chem., 1999, 42, p. 1088-1099). Accordingly, there continues to be considerable interest in the art with regard to the discovery of further selective inhibitors of PDE4s.

Successful results have already been obtained in the art with the discovery and development of selective PDE4 inhibitors. In vivo, PDE4 inhibitors reduce the influx of eosinophils to the lungs of allergen-challenged animals while also reducing the bronchoconstriction and elevated bronchial responsiveness occurring after allergen challenge. PDE4 inhibitors also suppress the activity of immune cells (including CD4⁺ T-lymphocytes, monocytes, mast cells, and basophils), reduce pulmonary edema, inhibit excitatory nonadrenergic noncholinergic neurotransmission (eNANC), potentiate inhibitory nonadrenergic noncholinergic neurotransmission (iNANC), reduce airway smooth muscle mitogenesis, and induce bronchodilation. PDE4 inhibitors also suppress the activity of a number of inflammatory cells associated with the pathophysiology of COPD, including monocytes/macrophages, CD4⁺ T-lymphocytes, eosinophils and neutrophils. PDE4 inhibitors also reduce vascular smooth muscle mitogenesis and potentially interfere with the ability of airway epithelial cells to generate pro-inflammatory mediators. Through the release of neutral proteases and acid hydrolases from their granules, and the generation of reactive oxygen species, neutrophils contribute to the tissue destruction associated with chronic inflammation, and are further implicated in the pathology of conditions such as emphysema. Therefore, PDE4 inhibitors are particularly useful for the treatment of a great number of inflammatory, respiratory and allergic diseases, disorders or conditions and for wounds and some of them are in clinical development mainly for treatment of asthma, COPD, bronchitis and emphysema.

The effects of PDE4 inhibitors on various inflammatory cell responses can be used as a basis for profiling and selecting inhibitors for further study. These effects include elevation of cAMP and inhibition of superoxide production, degranulation, chemotaxis, and tumor necrosis factor alpha (TNFa) release in eosinophils, neutrophils and monocytes.

Some nicotinamide derivatives having a PDE4 inhibitory activity have already been synthetized. For example, the patent application WO 98/45268 discloses nicotinamide derivatives having activity as selective inhibitors of PDE4D isozyme.

The patent applications WO 01/57036 and WO 03/068235 also disclose nicotinamide derivatives which are PDE4 inhibitors useful in the treatment of various inflammatory allergic and respiratory diseases and conditions.

However, there is still a huge need for additional PDE4 inhibitors that are good drug candidates. In particular, preferred compounds should bind potently to the PDE4 enzyme whilst showing little affinity for other receptors and enzymes. They should also possess favourable pharmacokinetic and metabolic activities, be non-toxic and demonstrate few side effects. Furthermore, it is also desirable that the ideal drug candidate will exist in a physical form that is stable and easily formulated.

The present invention therefore provides new nicotinamide derivatives of formula (I): 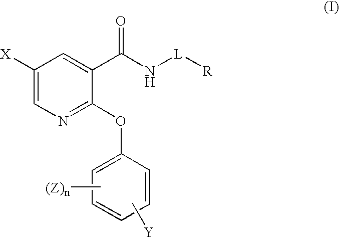
wherein:
X is hydrogen, methyl or halo,
Y is attached to the 3- or 4-position on the phenyl ring, and is S(O)ₚR¹, wherein R¹ is (C₁-C₄) alkyl optionally substituted by (C₃-C₆)cycloalkyl and p is 0, 1 or 2,
n is 1 or 2,
Z is selected from hydrogen, (C₁-C₃)alkyl, halo and (C₁-C₃)alkoxy, each Z being independently selected when n is 2,
or Y and (Z)ₙ, when attached to adjacent carbon atoms at the 3- or 4-position on the phenyl ring, are taken together with the carbon atoms to which they are attached to form a dihydrothienyl or dihydro-1,4-oxathiinyl ring,
L is a 5- or 6-membered heterocyclic ring containing one or two nitrogen ring atoms, which ring is optionally substituted by OH, (C₁-C₃)alkyl (optionally substituted by OH or by (C₁-C₃)alkoxy), halo or by (C₁-C₃)alkoxy,
R is H, (C₁-C₆)alkyl (optionally substituted by OH or (C₁-C₃)alkoxy), SO₂(C₁-C₃)alkyl), or COR²,
R² is selected from the group consisting of:
   (C₁-C₆)alkyl (optionally substituted by (C₁-C₃)alkoxy, OH, NR³R⁴, a 5- or 6-membered heterocyclic ring containing 1, 2 or 3 hetero ring atoms independently selected from N, O and S, OC(O)(C₁-C₃)alkyl) or S(O)ₚ(C₁-C₃)alkyl),
   (C₃-C₆)cycloalkyl,
   a 5- or 6-membered heterocyclic ring containing 1, 2 or 3 hetero ring atoms independently selected from N, O and S, which ring is optionally substituted by (C₁-C₃)alkyl (optionally substituted by OH), halo, ═O, OH, or by (C₁-C₃)alkoxy and
   phenyl, optionally substituted by 1, 2 or 3 substituents independently selected from OH, halo, (C₁-C₃)alkyl, (C₁-C₃)alkoxy, CO₂H, CO₂(C₁-C₃)alkyl) and OC(═O)(C₁-C₃)alkyl,
R³ and R⁴ are each independently selected from H, (C₁-C₃)alkyl and C(O)(C₁-C₃)alkyl),
and the pharmaceutically acceptable salts and solvates thereof,
with the proviso that the nicotinamide derivative is not
   (i) 5-methyl-2-(3-methylsulphonyl)-N-(pyrazin-5-yl)nicotinamide;
   (ii) 5-methyl-2-(3-ethylsulphonyl)-N-(pyrazin-5-yl)nicotinamide;
   (iii) 2-(3-methylsulphonyl)-N-(pyrazin-5-yl)nicotinamide; or
   (iv) 2-(3-ethylsulphonyl)-N-(pyrazin-5-yl)nicotinamide.

It has been found that these nicotinamide derivatives are inhibitors of PDE4 isoenzymes, particularly useful for the treatment of inflammatory, respiratory and allergic diseases and conditions or for wounds by showing excellent therapeutic utility and therapeutic index.

Preferably X is F and/or p is 0.

According to an aspect of the invention, Y is attached to the 3-position on the phenyl ring, and/or Y is S(O)ₚCH₃, S(O)ₚC₂H₅ or S(O)ₚCH₂(cyclopropyl). More preferably, Y is SCH₃, SC₂H₅ or SCH₂(cyclopropyl). Still more preferably, Y is SCH₃. Yet more preferably Y is 3-SCH₃.

According to another aspect of the invention, when (Z)ₙ is not H or F, it is attached to the 3-, 4-, and/or 5-position on the phenyl ring. Preferably, (Z)ₙ is H.

According to further aspect of the invention, L is a piperidine, pyrrolidine, pyrazine, pyridine or pyrimidine ring, which ring is optionally substituted by OH, methoxy, hydroxymethyl, ethoxy, or methyl. More preferably, L is piperidin-1,3-ylene, piperidin-1,4-ylene, pyrazin-5,1-ylene, 3-hydroxypyridin-6,4-ylene, pyridin-4,2-ylene, pyridin-2,6-ylene, pyridin-4,6-ylene, pyridin-3,6-ylene, 3-methoxypyridin-6,4-ylene, 2-methoxypyridin-5,3-ylene, 2-methoxypyridin-3,5-ylene, 3-ethoxypyridin-2,6-ylene, 3-hydroxymethylpyridin-2,6-ylene, 2-methylpyridin-3,6-ylene, 3-methylpyridin-4,6-ylene, 4-hydroxypirimidin-2,5-ylene or 4-hydroxypyrimidin-5,2-ylene, where the first number of the linkage indicates the attachment to the NH of the nicotinamide moiety, and the second number of the linkage is attached to the R moiety. Still more preferably, L is piperidin-1,4-ylene, pyrazin-5,1-ylene, 3-hydroxypyridin-6,4-ylene, pyridin-4,2-ylene, pyridin-2,6-ylene, pyrid in-4,6-ylene, pyridin-3,6-ylene, 2-methoxypyridin-3,5-ylene, 3-hydroxymethylpyridin-2,6-ylene or 3-methylpyridin-4,6-ylene, where the first number of the linkage indicates the attachment to the NH of the nicotinamide moiety, and the second number of the linkage is attached to the R moiety. Yet more preferably, L is piperidin-1,4-ylene, where the first number of the linkage indicates the attachment to the NH of the nicotinamide moiety, and the second number of the linkage is attached to the R moiety.

According to another aspect of the invention, R is attached to a nitrogen atom on the ring L. More preferably, R is H, (C₁-C₃)alkyl, SO₂(C₁-C₃)alkyl), or COR², wherein R² is (C₁-C₃)alkyl (optionally substituted by (C₁-C₃)alkoxy, OH, NR³R⁴ or a 5- or 6-membered heterocyclic ring containing 1, 2 or 3 hetero ring atoms independently selected from N, O and S),
cyclopropyl,
a 5- or 6-membered heterocyclic ring containing 1, 2 or 3 hetero ring atoms independently selected from N, O and S, which ring is optionally substituted by (C₁-C₃)alkyl (optionally substituted by OH), halo, ═O, OH, or by (C₁-C₃)alkoxy, or
phenyl, optionally substituted by 1, 2 or 3 substituents independently selected from OH, halo, (C₁-C₃)alkyl, (C₁-C₃)alkoxy, CO₂H, CO₂(C₁-C₃)alkyl) and OC(═O)(C₁-C₃)alkyl.

Still more preferably, R is H, CH₃, C₂H₅, COCH₃, SO₂CH₃, COC₂H₄(pyridyl), COC₂H₅, CO(cyclopropyl), COCH₂OH, CO(2-hydroxy-4-methylphenyl), CO(2-hydroxy-4-methoxyphenyl) or CO(2-hydroxyphenyl). Yet more preferably, R is H, COCH₃ or SO₂CH₃. Most preferably R is H or COCH₃.

In the here above general formula (1), halo denotes a halogen atom selected from the group consisting of fluoro, chloro, bromo and iodo in particular fluoro or chloro.

(C₁-C₃)alkyl, (C₁-C₄)alkyl or (C₁-C₆)alkyl radicals denote a straight-chain or branched group containing respectively 1 to 3, 1 to 4 and 1 to 6 carbon atoms. This also applies if they carry substituents or occur as substituents of other radicals, for example in (C₁-C₄)alkoxy radicals, (C₁-C₄)thioalkyl radicals, (C₁-C₄)haloalkyl radicals, hydroxy(C₁-C₄)alkyl radicals, C(═O)O(C₁-C₄)alkyl radicals etc. Examples of suitable (C₁-C₃)alkyl, (C₁-C₄)alkyl and (C₁-C₆)alkyl radicals are methyl, ethyl, n-propyl, iso-propyl, n-butyl, iso-butyl, sec-butyl, tert-butyl, pentyl and hexyl. Examples of suitable (C₁-C₄)alkoxy radicals are methoxy, ethoxy, n-propyloxy, iso-propyloxy, n-butyloxy, iso-butyloxy, sec-butyloxy and tert-butyloxy. Examples of suitable (C₁-C₄)thioalkyl radicals are thiomethyl, thioethyl, thio-n-propyl, thio-iso-propyl, thio-n-butyl, thio-iso-butyl, thio-sec-butyl and thio-tert-butyl.

(C₃-C₆)cycloalkyl radicals represent 3-membered to 6-membered saturated monocyclic rings. Examples of suitable (C₃-C₆)cycloalkyl radicals are in particular cyclopropyl, cyclobutyl, cyclopentyl and cyclohexyl. These radicals can be optionally substituted.

In the hereabove general formula (1), a heterocyclic ring is a radical of a monocyclic or polycyclic aromatic system having 5 to 14 ring members, which contains 1, 2 or 3 heteroatom(s) depending in number and quality of the total number of ring members, selected from nitrogen (N), oxygen (O) and sulphur (S). If several heteroatoms are contained, these can be identical or different. Heterocyclic rings can also be unsubstituted, monosubstituted or polysubstituted, as indicated in the definition of R2 hereabove for general formula (1) according to the present invention. Examples of suitable heterocyclic radicals are the radicals derivated from piperidine, pyrrolidine, pyrazine, pyridine or pyrimidine ring, pyrrole, furan, furazan, thiophene, imidazole, pyrazole, oxazole, isoxazole, thiazole, isothiazole, tetrazole, triazine, pyridine, pyrazine, pyrimidine, pyridazine, indolizine, indole, isoindole, indazole, purine, naphthyridine, phthalazine, quinoline, isoquinoline, quinoxaline, quinazoline, cinnoline, and benzo-fused derivatives of these heteroaryls, such as for example benzofuran, benzothiophene, benzoxazole, and benzothiazole. Particularly preferred are the heteroaryl radicals selected from pyrrolyl, pyrazolyl, 1,2,3-triazolyl, 1,2,4-triazolyl, tetrazolyl, oxazolyl, isoxazolyl, thiazolyl, isothiazolyl, 1,2,4-oxadiazolyl, 1,3,4-oxadiazolyl, furanyl, thienyl, pyridinyl, pyridazinyl, pyrimidinyl, and pyrazinyl.

In the general formula (1) according to the present invention, when a radical is mono- or poly-substituted, said substituent(s) can be located at any desired position(s).

Also, when a radical is polysubstituted, said substituents can be identical or different, unless otherwise stated.

The nicotinamide derivatives of the formula (1) can be prepared using conventional procedures such as by the following illustrative methods in which X, L, Y, Z and R are as previously defined for the nicotinamide derivatives of the formula (1) unless otherwise stated.

In the Methods below, unless otherwise specified, the substituents are as defined above with reference to the compounds of formula (I) above.

Method A

The compounds of formula (I) may be made by reaction of the corresponding nicotinic acid (II) with a compound of formula NH₂—L—R 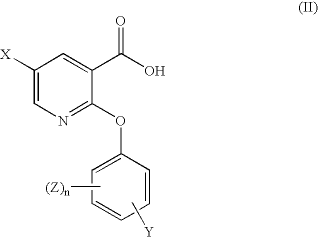
suitably in the presence of an acid/amine, or peptide, coupling agent. The reaction may suitably be carried by reaction of the acid with carbonyldiimidazole in a suitable inert solvent such as dichloromethane, followed by addition of the compound NH₂—L—R, suitably in the presence of a base such as 4-dimethylaminopyridine, as exemplified in Examples 1-4 below.

An alternative method starting from acids (II) is to use a suitable diimide such as 1-(3-dimethylaminopropyl)-3-ethylcarbodiimide in conjunction with an agent such as 1-hydroxybenzotriazole. The acid (II) may be added to the mixture in an inert solvent such as dichloromethane, followed by addition of the amine NH₂—L—R. This reaction type, reagents, conditions, solvents and variations thereof are exemplified in Examples 57-69 below.

Other suitable regimes for the transformation (II) to (I) may be found in the volume by Larock, supra, pp972-976.

The acids of formula (II) and amines of formula NH₂—L—R may be commercially available, or can be made using the methods described herein, including in the Preparations below, the art mentioned herein, or routine adaptation thereof.

Method B

Compounds of formula (I) may be made by a coupling reaction of a chloro-compound of formula (III) with a phenol of formula (IV).

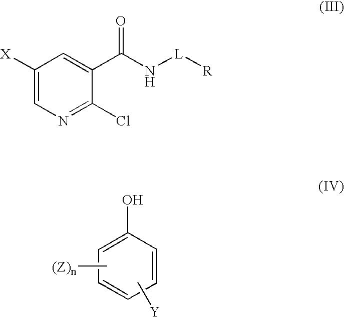

Suitably the phenol (IV) and chloro-compound (III) are mixed with caesium carbonate in an inert solvent such as toluene:N-methylpyrrolidine at ambient temperature, followed by addition of copper (I) iodide and then heating to a suitable temperature such as 110° C.

This reaction type, reagents, conditions, solvents and variations thereof are exemplified in Examples 40-55 below.

The chloro-compounds (III) and the phenols (IV) may be commercially available, or can be made using the methods described herein, including in the Preparations below, the art mentioned herein, or routine adaptation thereof.

Certain compounds of formula (I) may be transformed into other compounds of formula (I) by suitable functional group interconversion (FGI) of a type well-known to those skilled in the art.

For examples where the compound of formula (I) contains an ester or acid moiety, these can be interconverted readily by known hydrolysis or esterification methods respectively.

For examples where compounds of formula (I) where R is H are transformed into compounds of formula (I) where R is not H, see Examples 5, 9-39 (R=carbonyl-linked moiety), 6-8 (R=alkyl), 56 (R=SO₂(alkyl)).

For examples of compounds of formula (I) where the Y moiety is a thioalkyl moiety is oxidised to a compound of formula (I) where Y is a sulphoxyl or sulphonyl moiety, see Examples 71, 72.

Many other FGIs of compounds of formula (I) into other compounds of formula (I) are possible using methods exemplified in the Examples and Preparations sections below, and standard FGI chemistry known in the art.

It will be apparent to those skilled in the art that other protection and subsequent deprotection regimes during synthesis of a compound of the invention may be achieved by conventional techniques, for example as described in the volumes by Greene and Wuts, and Kocienski, supra, some of which are mentioned specifically herein.

All of the above reactions and the preparations of novel starting materials using in the preceding methods are conventional and appropriate reagents and reaction conditions for their performance or preparation as well as procedures for isolating the desired products will be well-known to those skilled in the art with reference to literature precedents and the examples and preparations hereto.

For some of the steps of the here above described process of preparation of the nicotinamide derivatives of formula (1), it can be necessary to protect the potential reactive functions that are not wished to react. In such a case, any compatible protecting radical can be used. In particular methods such as those described by T. W. GREENE (Protective Groups in Organic Synthesis, A. Wiley-Interscience Publication, 1981) or by McOMIE (Protective Groups in Organic Chemistry, Plenum Press, 1973), can be used.

Also, the nicotinamide derivatives of formula (1) as well as intermediate for the preparation thereof can be purified according to various well-known methods, such as for example crystallization or chromatography.

According to a first aspect, particularly preferred are nicotinamide derivatives of the formula (1) in which X is F. Further preferred are nicotinamide derivatives wherein p is 0.

Y is preferably attached to the 3-position on the phenyl ring, and represents in particular S(O)ₚCH₃, S(O)ₚC₂H₅ or S(O)ₚCH₂(cyclopropyl), preferably SCH₃, SC₂H₅ or SCH₂(cyclopropyl), more preferably SCH₃, most preferably 3-SCH₃.

When (Z)ₙ is not H or F, it is preferably attached to the 3-, 4-, and/or 5-position on the phenyl ring. (Z)ₙ is preferably H.

L is preferably a piperidine, pyrrolidine, pyrazine, pyridine or pyrimidine ring, which ring is optionally substituted by OH, methoxy, hydroxymethyl, ethoxy, or methyl, in particular piperidin-1,3-ylene, piperidin-1,4-ylene, pyrazin-5,1-ylene, 3-hydroxypyridin-6,4-ylene, pyridin-4,2-ylene, pyridin-2,6-ylene, pyridin-4,6-ylene, pyridin-3,6-ylene, 3-methoxypyridin-6,4-ylene, 2-methoxypyridin-5,3-ylene, 2-methoxypyridin-3,5-ylene, 3-ethoxypyridin-2,6-ylene, 3-hydroxymethylpyridin-2,6-ylene, 2-methylpyridin-3,6-ylene, 3-methylpyridin-4,6-ylene, 4-hydroxypirimidin-2,5-ylene or 4-hydroxypyrimidin-5,2-ylene, where the first number of the linkage indicates the attachment to the NH of the nicotinamide moiety, and the second number of the linkage is attached to the R moiety.

More preferably L is piperidin-1,4-ylene, pyrazin-5,1-ylene, 3-hydroxypyridin-6,4-ylene, pyridin-4,2-ylene, pyridin-2,6-ylene, pyridin-4,6-ylene, pyridin-3,6-ylene, 2-methoxypyridin-3,5-ylene, 3-hydroxymethyl pyridin-2,6-ylene or 3-methylpyridin-4,6-ylene, where the first number of the linkage indicates the attachment to the NH of the nicotinamide moiety, and the second number of the linkage is attached to the R moiety, most preferably L is piperidin-1,4-ylene, where the first number of the linkage indicates the attachment to the NH of the nicotinamide moiety, and the second number of the linkage is attached to the R moiety.

R is preferably attached to a nitrogen atom on the ring L and represents R H, (C₁-C₃)alkyl, SO₂(C₁-C₃)alkyl), or COR²,
wherein R² is (C₁-C₃)alkyl (optionally substituted by (C₁-C₃)alkoxy, OH, NR³R⁴ or a 5- or 6-membered heterocyclic ring containing 1, 2 or 3 hetero ring atoms independently selected from N, O and S),
cyclopropyl,
a 5- or 6-membered heterocyclic ring containing 1, 2 or 3 hetero ring atoms independently selected from N, O and S, which ring is optionally substituted by (C₁-C₃)alkyl (optionally substituted by OH), halo, ═O, OH, or by (C₁-C₃)alkoxy, or phenyl, optionally substituted by 1, 2 or 3 substituents independently selected from OH, halo, (C₁-C₃)alkyl, (C₁-C₃)alkoxy, CO₂H, CO₂(C₁-C₃)alkyl) and OC(═O)(C₁-C₃)alkyl.

More preferably R is H, CH₃, C₂H₅, COCH₃, SO₂CH₃, COC₂H₄(pyridyl), COC₂H₅, CO(cyclopropyl), COCH₂OH, CO(2-hydroxy4-methylphenyl), CO(2-hydroxy-4-methoxyphenyl) or CO(2-hydroxyphenyl), in particular H, COCH₃ or SO₂CH₃. Most preferably R is H or COCH₃.

Particularly preferred examples of the nicotinamide derivatives of the formula (1) are as described in the Examples section hereafter.

The nicotinamide derivatives of formula (1) may also be optionally transformed in pharmaceutically acceptable salts. In particular, these pharmaceutically acceptable salts of the nicotinamide derivatives of the formula (1) include the acid addition and the base salts (including disalts) thereof.

Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodie, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, 1-hydroxy-2-naphtoate, 3-hydroxy-2-naphthoate and tosylate saltes.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see Stahl and Wermuth, Handbook of Pharmaceutical Salts: Properties, Selection and Use, Wiley-VCH, Weinheim, Germany (2002).

A pharmaceutically acceptable salt of a nicotinamide derivative of the formula (1) may be readily prepared by mixing together solutions of the nicotinamide derivative of formula (1) and the desired acid or base, as appropriate. The salt may precipitate from solution and be collected by filtration or may be recovered by evaporation of the solvent.

Pharmaceutically acceptable solvates in accordance with the invention include hydrates and solvates wherein the solvent of crystallization may be isotopically substituted, e.g. D2O, d₆-acetone, d₆-DMSO.

Also within the scope of the invention are clathrates, drug-host inclusion complexes wherein, in contrast to the aforementioned solvates, the drug and host are are present in non-stoichiometric amounts. For a review of such complexes, see J Pharm Sci, 64 (8), 1269-1288 by Haleblian (August 1975).

Hereinafter all references to nicotinamide derivatives of formula (1) include references to salts thereof and to solvates and clathrates of compounds of formula (1) and salts thereof.

The invention includes all polymorphs of the nicotinamide derivatives of formula (1).

Also within the scope of the invention are so-called “prodrugs” of the nicotinamide derivatives of formula (1). Thus certain derivatives of nicotinamide derivatives of formula (1) which have little or no pharmacological activity themselves can, when metabolised upon administration into or onto the body, give rise to nicotinamide derivatives of formula (1) having the desired activity. Such derivatives are referred to as “prodrugs”.

Prodrugs in accordance with the invention can, for example, be produced by replacing appropriate functionalities present in the nicotinamide derivatives of formula (1) with certain moieties known to those skilled in the art as “pro-moieties” as described, for example, in “Design of Prodrugs” by H Bundgaard (Elsevier, 1985).

Finally, certain nicotinamide derivatives of formula (1) may themselves act as prodrugs of other nicotinamide derivatives of formula (1).

Nicotinamide derivatives of formula (1) containing one or more asymmetric carbon atoms can exist as two or more optical isomers. Where a nicotinamide derivative of formula (1) contains an alkenyl or alkenylene group, geometric cis/trans (or Z/E) isomers are possible, and where the nicotinamide derivative contains, for example, a keto or oxime group, tautomeric isomerism (‘tautomerism’) may occur. It follows that a single nicotinamide derivative may exhibit more than one type of isomerism.

Included within the scope of the present invention are all optical isomers, geometric isomers and tautomeric forms of the nicotinamide derivatives of formula (1), including compounds exhibiting more than one type of isomerism, and mixtures of one or more thereof.

Cis/trans isomers may be separated by conventional techniques well known to those skilled in the art, for example, fractional crystallisation and chromatography.

Conventional techniques for the preparation/isolation of individual stereoisomers include the conversion of a suitable optically pure precursor, resolution of the racemate (or the racemate of a salt or derivative) using, for example, chiral HPLC, or fractional crystallisation of diastereoisomeric salts formed by reaction of the racemate with a suitable optically active acid or base, for example, tartaric acid.

The present invention also includes all pharmaceutically acceptable isotopic variations of a nicotinamide derivative of formula (1). An isotopic variation is defined as one in which at least one atom is replaced by an atom having the same atomic number, but an atomic mass different from the atomic mass usually found in nature.

Examples of isotopes suitable for inclusion in the nicotinamide derivatives of the invention include isotopes of hydrogen, such as ²H and ³H, carbon, such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the nicotinamide derivative of formula (1) isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the nicotinamide derivatives of formula (1), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the nicotinamide derivatives of formula (1) can generally be prepared by conventional techniques known to those skilled in the art or by processes analogous to those described in the accompanying Examples and Preparations using appropriate isotopic variations of suitable reagents.

According to a further aspect, the present invention concerns mixtures of nicotinamide derivatives of the formula (1), as well as mixtures with or of their pharmaceutically acceptable salts, solvates, polymorphs, isomeric forms and/or isotope forms.

According to the present invention, all the here above mentioned forms of the nicotinamide derivatives of formula (1) except the pharmaceutically acceptable salts (i.e. said solvates, polymorphs, isomeric forms and isotope forms), are defined as “derived forms” of the nicotinamide derivatives of formula (1) in what follows.

The nicotinamide derivatives of formula (1), their pharmaceutically acceptable salts and/or derived forms, are valuable pharmaceutical active compounds, which are suitable for the therapy and prophylaxis of numerous disorders in which the PDE4 enzymes are involved, in particular the inflammatory disorders, allergic disorders, respiratory diseases and wounds.

The nicotinamide derivatives of formula (1) and their pharmaceutically acceptable salts and derived forms as mentioned above can be administered according to the invention to animals, preferably to mammals, and in particular to humans, as pharmaceuticals for therapy or prophylaxis. They can be administered per se, in mixtures with one another or in combination with other drugs, or in the form of pharmaceutical preparations which permit enteral (gastric) or parenteral (non-gastric) administration and which as active constituent contain an efficacious dose of at least one nicotinamide derivative of the formula (1), its pharmaceutically acceptable salts and/or derived forms, in addition to customary pharmaceutically innocuous excipients and/or additives. The term “excipient” is used herein to describe any ingredient other than the compound of the invention. The choice of excipient will to a large extent depend on the particular mode of administration.

The nicotinamide derivatives of formula (1), their pharmaceutically acceptable salts and/or derived forms may be freeze-dried, spray-dried, or evaporatively dried to provide a solid plug, powder, or film of crystalline or amorphous material. Microwave or radio frequency drying may be used for this purpose.

Oral Administration

The nicotinamide derivatives of formula (1) their pharmaceutically acceptable salts and/or derived forms of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The nicotinamide derivatives of formula (1), their pharmaceutically acceptable salts and/or derived forms of the invention may also be used in fast-dissolving, fast-disintegrating dosage forms such as those described in Expert Opinion in Therapeutic Patents, 11 (6), 981-986 by Liang and Chen (2001).

The composition of a typical tablet in accordance with the invention may comprise:

| Ingredient | % w/w |
|---|---|
| Nicotinamide derivative of formula (1) | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

Parenteral Administration

The nicotinamide derivatives of formula (1), their pharmaceutically acceptable salts and/or derived forms of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of nicotinamide derivatives of formula (1) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

Topical Administration

The nicotinamide derivatives of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus nicotinamide derivatives of formula (1) may be formulated in a more solid form for administration as an implanted depot providing long-term release of the active compound.

Inhaled/intranasal Administration

The nicotinamide derivatives of formula (1) can also be administered intranasally or by inhalation, typically in the form of a dry powder (either alone, as a mixture, for example, in a dry blend with lactose in anhydrous or monohydrate form, preferably monohydrate, mannitol, dextran, glucose, maltose, sorbitol, xylitol, fructose, sucrose or trehalose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 20 mg of the nicotinamide derivative of formula (1) per actuation and the actuation volume may vary from 1 μl to 100 μl. A typical formulation may comprise a nicotinamide derivative of formula (1), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the nicotinamide derivative of formula (1), a suitable powder base such as lactose or starch and a performance modifier such as I-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff” containing from 1 μg to 4000 μg of the nicotinamide derivative of formula (1). The overall daily dose will typically be in the range 1 μg to 20 mg which may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release. Sustained or controlled release can be obtained by using for example poly(D,L-lactic-co-glycolic acid).

Flavouring agents, such as methol and levomethol and/or sweeteners such as saccharing or saccharin sodium can be added to the formulation.

Rectal/Intravaginal Administration

The nicotinamide derivatives of formula (1) may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

Ocular/andial Administration

The nicotinamide derivatives of formula (1) may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

Enabling Technologies

The nicotinamide derivatives of formula (1) may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Dosage

For administration to human patients, the total daily dose of the nicotinamide derivatives of formula (1) is typically in the range 0.001 mg/kg to 100 mg/kg depending, of course, on the mode of administration. The total daily dose may be administered in single or divided doses. The physician will readily be able to determine doses for subjects depending on age, weight, health state and sex or the patient as well as the severity of the disease.

According to another embodiment of the present invention, the nicotinamide derivatives of the formula (1), their pharmaceutically acceptable salts and/or their derived forms, can also be used as a combination with one or more additional therapeutic agents to be co-administered to a patient to obtain some particularly desired therapeutic end result. The second and more additional therapeutic agents may also be a nicotinamide derivatives of the formula (1), their pharmaceutically acceptable salts and/or their derived forms, or one or more PDE4 inhibitors known in the art. More typically, the second and more therapeutic agents will be selected from a different class of therapeutic agents.

As used herein, the terms “co-administration”, “co-administered” and “in combination with”, referring to the nicotinamide derivatives of formula (1) and one or more other therapeutic agents, is intended to mean, and does refer to and include the following:
simultaneous administration of such combination of nicotinamide derivative(s) and therapeutic agent(s) to a patient in need of treatment, when such components are formulated together into a single dosage form which releases said components at substantially the same time to said patient,
substantially simultaneous administration of such combination of nicotinamide derivative(s) and therapeutic agent(s) to a patient in need of treatment, when such components are formulated apart from each other into separate dosage forms which are taken at substantially the same time by said patient, whereupon said components are released at substantially the same time to said patient,
sequential administration of such combination of nicotinamide derivative(s) and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of nicotinamide derivative(s) and therapeutic agent(s) to a patient in need of treatment, when such components are formulated together into a single dosage form which releases said components in a controlled manner whereupon they are concurrently, consecutively, and/or overlappingly administered at the same and/or different times by said patient.

Suitable examples of other therapeutic agents which may be used in combination with the nicotinamide derivatives of the formula (1), their pharmaceutically acceptable salts and/or their derived forms include, but are by no mean limited to:
(a) 5-Lipoxygenase (5-LO) inhibitors or 5-lipoxygenase activating protein (FLAP) antagonists,
(b) Leukotriene antagonists (LTRAs) including antagonists of LTB4, LTC4, LTD4, and LTE4,
(c) Histaminic receptor antagonists including H1, H3 and H4 antagonists,
(d) α1- and α2-adrenoceptor agonist vasoconstrictor sympathomimetic agents for decongestant use,
(e) Muscarinic M3 receptor antagonists or anticholinergic agents,
(f) β2-adrenoceptor agonists,
(g) Theophylline,
(h) Sodium cromoglycate,
(i) COX-1 inhibitors (NSAIDs) and COX-2 selective inhibitors,
(j) Oral or inhaled Glucocorticosteroids,
(k) Monoclonal antibodies active against endogenous inflammatory entities,
(l) Anti-tumor necrosis factor (anti-TNF-a) agents,
(m) Adhesion molecule inhibitors including VLA-4 antagonists,
(n) Kinin-B1- and B2-receptor antagonists,
(o) Immunosuppressive agents,
(p) Inhibitors of matrix metalloproteases (MMPs),
(q) Tachykinin NK1, NK2 and NK3 receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFkb pathway, e.g. IKK inhibitors,
(w) Agents that can be classed as mucolytics or anti-tussive,
(x) antibiotics, and
(y) p38 MAP kinase inhibitors

According to the present invention, combination of the nicotinamide derivatives of formula (1) with:
muscarinic M3 receptor agonists or anticholinergic agents including in particular ipratropium salts, namely bromide, tiotropium salts, namely bromide, oxitropium salts, namely bromide, perenzepine, and telenzepine,
β2-adrenoceptor agonists including albutarol, salbutamol, formoterol and salmeterol,
p38 MAP kinase inhibitors,
H3 antagonists,
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, and mometasone furoate,
or adenosine A2a receptor agonists,
   are preferred.

It is to be appreciated that all references herein to treatment include curative, palliative and prophylactic treatment. The description which follows concerns the therapeutic applications to which the nicotinamide derivatives of formula (1) may be put.

The nicotinamide derivatives of formula (1) inhibit the PDE4 isozyme and thereby have a wide range of therapeutic applications, as described further below, because of the essential role, which the PDE4 family of isozymes plays in the physiology of all mammals. The enzymatic role performed by the PDE4 isozymes is the intracellular hydrolysis of adenosine 3′,5′-monophosphate (cAMP) within pro-inflammatory leukocytes. cAMP, in turn, is responsible for mediating the effects of numerous hormones in the body, and as a consequence, PDE4 inhibition plays a significant role in a variety of physiological processes. There is extensive literature in the art describing the effects of PDE inhibitors on various inflammatory cell responses, which in addition to cAMP increase, include inhibition of superoxide production, degranulation, chemotaxis and tumor necrosis factor (TNF) release in eosinophils, neutrophils and monocytes.

Therefore, a further aspect of the present invention relates to the use of the nicotinamide derivatives of formula (1), their pharmaceutically acceptable salts and/or derived forms, in the treatment of diseases, disorders, and conditions in which the PDE4 isozymes are involved. More specifically, the present invention also concerns the use of the nicotinamide derivatives of formula (1), their pharmaceutically acceptable salts and/or derived forms, in the treatment of diseases, disorders, and conditions selected from the group consisting of:
asthma of whatever type, etiology, or pathogenesis, in particular asthma that is a member selected from the group consisting of atopic asthma, non-atopic asthma, allergic asthma, atopic bronchial IgE□mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy
pneumoconiosis of whatever type, etiology, or pathogenesis, in particular pneumoconiosis that is a member selected from the group consisting of aluminosis or bauxite workers' disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,
seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,
rheumatoid arthritis of whatever type, etiology, or pathogenesis, in particular rheumatoid arthritis that is a member selected from the group consisting of acute arthritis, acute gouty arthritis, chronic inflammatory arthritis, degenerative arthritis, infectious arthritis, Lyme arthritis, proliferative arthritis, psoriatic arthritis and vertebral arthritis,
a gout, and fever and pain associated with inflammation,
an eosinophil-related disorder of whatever type, etiology, or pathogenesis, in particular an eosinophil-related disorder that is a member selected from the group consisting of eosinophilia, pulmonary infiltration eosinophilia, Loffier's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrotizing vasculitis,
atopic dermatitis, allergic dermatitis, contact dermatitis, or allergic or atopic eczema,
urticaria of whatever type, etiology, or pathogenesis, in particular urticaria that is a member selected from the group consisting of immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria and papular urticaria,
conjunctivitis of whatever type, etiology, or pathogenesis, in particular conjunctivitis that is a member selected from the group consisting of actinic conjunctivitis, acute catarrhal conjunctivitis, acute contagious conjunctivitis, allergic conjunctivitis, atopic conjunctivitis, chronic catarrhal conjunctivitis, purulent conjunctivitis and vernal conjunctivitis,
uveitis of whatever type, etiology, or pathogenesis, in particular uveitis that is a member selected from the group consisting of inflammation of all or part of the uvea, anterior uveitis, iritis, cyclitis, iridocyclitis, granulomatous uveitis, nongranulomatous uveitis, phacoantigenic uveitis, posterior uveitis, choroiditis; and chorioretinitis,
psoriasis;
multiple sclerosis of whatever type, etiology, or pathogenesis, in particular multiple sclerosis that is a member selected from the group consisting of primary progressive multiple sclerosis and relapsing remitting multiple sclerosis,
autoimmune/inflammatory diseases of whatever type, etiology, or pathogenesis, in particular an autoimmune/inflammatory disease that is a member selected from the group consisting of autoimmune hematological disorders, hemolytic anemia, aplastic anemia, pure red cell anemia, idiopathic thrombocytopenic purpura, systemic lupus erythematosus, polychondritis, scleroderma, Wegner's granulomatosis, dermatomyositis, chronic active hepatitis, myasthenia gravis, Stevens-Johnson syndrome, idiopathic sprue, autoimmune inflammatory bowel diseases, ulcerative colitis, endocrin opthamopathy, Grave's disease, sarcoidosis, alveolitis, chronic hypersensitivity pneumonitis, primary biliary cirrhosis, juvenile diabetes or diabetes mellitus type I, keratoconjunctivitis sicca, epidemic keratoconjunctivitis, diffuse interstitial pulmonary fibrosis or interstitial lung fibrosis, idiopathic pulmonary fibrosis, cystic fibrosis, glomerulonephritis with and without nephrotic syndrome, acute glomerulonephritis, idiopathic nephrotic syndrome, minimal change nephropathy, inflammatory/hyperproliferative skin diseases, benign familial pemphigus, pemphigus erythematosus, pemphigus foliaceus, and pemphigus vulgaris,
prevention of allogeneic graft rejection following organ transplantation,
inflammatory bowel disease (IBD) of whatever type, etiology, or pathogenesis, in particular inflammatory bowel disease that is a member selected from the group consisting of collagenous colitis, colitis polyposa, transmural colitis, ulcerative colitis and Crohn's disease (CD),
septic shock of whatever type, etiology, or pathogenesis, in particular septic shock that is a member selected from the group consisting of renal failure, acute renal failure, cachexia, malarial cachexia, hypophysial cachexia, uremic cachexia, cardiac cachexia, cachexia suprarenalis or Addison's disease, cancerous cachexia and cachexia as a consequence of infection by the human immunodeficiency virus (HIV),
liver injury,
pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension,
bone loss diseases, primary osteoporosis and secondary osteoporosis,
central nervous system disorders of whatever type, etiology, or pathogenesis, in particular a central nervous system disorder that is a member selected from the group consisting of depression, Alzheimers disease, Parkinson's disease, learning and memory impairment, tardive dyskinesia, drug dependence, arteriosclerotic dementia and dementias that accompany Huntington's chorea, Wilson's disease, paralysis agitans, and thalamic atrophies,
infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex,
yeast and fungus infections wherein said yeast and fungi are sensitive to upregulation by TNF-α or elicit TNF-α production in their host, e.g., fungal meningitis, particularly when administered in conjunction with other drugs of choice for the treatment of systemic yeast and fungus infections, including but are not limited to, polymixins, e.g. Polymycin B, imidazoles, e.g. clotrimazole, econazole, miconazole, and ketoconazole, triazoles, e.g. fluconazole and itranazole as well as amphotericins, e.g. Amphotericin B and liposomal Amphotericin B,
ischemia-reperfusion injury, ischemic heart disease, autoimmune diabetes, retinal autoimmunity, chronic lymphocytic leukemia, HIV infections, lupus erythematosus, kidney and ureter disease, urogenital and gastrointestinal disorders and prostate diseases,
reduction of scar formation in the human or animal body, such as scar formation in the healing of acute wounds, and
psoriasis, other dermatological and cosmetic uses, including antiphlogistic, skin-softening, skin elasticity and moisture-increasing activities.

According to one aspect the present invention relates in particular to the treatment of a respiratory disease, such as adult respiratory distress syndrome (ARDS), bronchitis, chronic obstructive pulmonary disease (COPD), cystic fibrosis, asthma, emphysema, bronchiectasis, chronic sinusitis and rhinitis.

According to another aspect the present invention relates in particular to the treatment of gastrointestinal (GI) disorders, in particular inflammatory bowel diseases (IBD) such as Crohn's disease, ileitis, collagenous colitis, colitis polyposa, transmural colitis and ulcerative colitis.

According to a further aspect the present invention relates also to the reduction of scars formation.

A still further aspect of the present invention also relates to the use of the nicotinamide derivatives of formula (1), their pharmaceutically acceptable salts and/or derived forms, for the manufacture of a drug having a PDE4 inhibitory activity. In particular, the present inventions concerns the use of the nicotinamide derivatives of formula (1), their pharmaceutically acceptable salts and/or derived forms, for the manufacture of a drug for the treatment of inflammatory, respiratory, allergic and scar-forming diseases, disorders, and conditions, and more precisely for the treatment of diseases, disorders, and conditions that are listed above.

As a consequence, the present invention provides a particularly interesting method of treatment of a mammal, including a human being, with a PDE4 inhibitor including treating said mammal with an effective amount of a nicotinamide derivative of formula (1), its pharmaceutically acceptable salts and/or derived forms. More precisely, the present invention provides a particularly interesting method of treatment of a mammal, including a human being, to treat an inflammatory, respiratory, allergic and scar-forming disease, disorder or condition, including treating said mammal with an effective amount of a nicotinamide derivative of formula (1), its pharmaceutically acceptable salts and/or derived forms.

The following examples illustrate the preparation of the nicotinamide derivatives of the formula (1):

### EXAMPLES AND PREPARATIONS

Melting points were determined using open glass capillary tubes and a Gallenkamp melting point apparatus and are uncorrected. Nuclear magnetic resonance (NMR) data were obtained using Varian Unity Inova-400, Varian Unity Inova-300 or Bruker AC300 spectrometers and are quoted in parts per million from tetramethylsilane. Mass spectral (MS) data were obtained on a Finnigan Mat. TSQ 7000 or a Fisons Instruments Trio 1000. The calculated and observed ions quoted refer to the isotopic composition of lowest mass. Infra red (IR) spectra were measured using a Nicolet Magna 550 Fourier transform infra-red spectrometer. Flash chromatography refers to column chromatography on silica gel (Kieselgel 60, 230-400 mesh, from E. Merck, Darmstadt. Kieselgel 60 F₂₅₄ plates from E. Merck were used for TLC, and compounds were visualised using UV light, 5% aqueous potassium permanganate or Dragendorff's reagent (oversprayed with aqueous sodium nitrite). Thermal analyses by Differential Scanning Calorimetry (DSC) and ThermoGravimetric Analysis (TGA) were obtained using Perkin Elmer DSC7 and TGA7. Moisture sorption characteristics were recorded using Surface Measurement Systems Ltd. Automated Water Sorption Analyser DVS 1. Water content was determined on a Mitsubishi CA100 (Coulometric Karl Fisher Titrator). Powder X-ray diffraction (PXRD) pattern was determined using a Siemens D5000 powder X-ray diffractometer fitted with an automatic sample changer, a theta-theta goniometer, automatic beam divergence slits, a secondary monochromator and a scintillation counter. Other measurements were taken using standard equipment. Hexane refers to a mixture of hexanes (hplc grade) b.p. 65-70° C. “Ether” and “Et₂O” refers to diethyl ether. Acetic acid refers to glacial acetic acid. 1-Hydroxy-7-aza-1H-1,2,3-benzotriazole (HOAt). “HOBt” is 1-hydroxy-1H-1,2,3-benzotriazole. N-[(dimethylamino)-1H-1,2,3-triazolo[4,5-b]pyridin-1-ylmethylene]-N-methylmethaninium hexafluorophosphate N-oxide (HATU) and 7-azabenzotriazol-1-yloxytris(pyrrolidino)phosphonium hexafluorophosphate (PyAOP) were purchased from PerSeptive Biosystems U.K. Ltd. “DIPE” refers to diisopropyl ether. Reverse-phase silica gel for flash chromatography was obtained from Fluka (Fluka 100, C₁₈, 40-63μ). “DCM” is dichloromethane. “THF” is tetrahydrofuran. “WSCDI” is 1-(3-dimethylaminopropyl)-3-ethylcarbodiimide hydrochloride. EtOAc” is ethyl acetate. “MeOH” is methanol. “DMSO” is dimethylsulphoxide. “ACE-Cl” is 1-chloroethyl chloroformate. “NMM” is N-methylmorpholine. “Pentane” refers to High Performance Liquid Chromatography (HPLC) grade n-pentane (b.pt.35-37° C.). Nomenclature has been allocated using the commercially available ACD program. Standard abbreviations are used throughout, e.g. “Me” is methyl, “Et” is ethyl, “Pr” is propyl, “Ph” is phenyl, etc.

^{a}HPLC autopurification performed using 2 columns—Phenomenex LUNA C8 150×21.2 mm, 10 μm and Phenomenex MAGELLEN C18 150×21.2 mm, 5 μm, eluting with a gradient system of organic solvent [ammonium acetate (aq) 100 mM: acetonitrile (1:9)]: aqueous solvent [ammonium acetate (aq) 100 mM: acetonitrile (9:1)]

^{b}HPLC autopurification performed using 2 columns—Phenomenex LUNA C8 150×21.2 mm, 10 μm and Phenomenex MAGELLEN C18 150×21.2 mm, 5 μm, eluting with a gradient system of organic solvent (acetonitrile):aqueous solvent (0.1% aqueous trifluoroacetic acid)

Preparation 1

1-Methoxy-3-methylsulfanyl-benzene

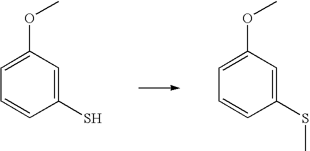

Methyl iodide (14.67 ml, 0.235 mol) was added dropwise to a solution of 3-methoxy-benzenethiol (30 g, 0.214 mol) and potassium carbonate (29.6 g, 0.214 mol) in acetone (400 ml) under nitrogen at 3° C. The reaction was allowed to warm to room temperature and the solvent was removed under reduced pressure. The residue was diluted with water (300 ml) and the aqueous layer was extracted with diethylether (3×200 ml). The combined organic extracts were dried over MgSO₄ and the solvent was removed under reduced pressure to give 1-Methoxy-3-methylsulfanyl-benzene (32.87 g) as a pale yellow liquid.

¹H NMR (400 MHz, CDCl₃): δ=7.17-7.21 (1H, t), 6.83-6.86 (1H, d), 6.81 (1H, s), 6.65-6.69 (1H, m), 3.80 (3H, s), 2.46 (3H, s) ppm.

Preparation 2

3-Methylsulfanyl-phenol

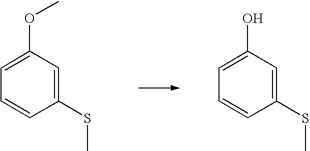

1-Methoxy-3-methylsulfanyl-benzene (32.87 g, 0.213 mol) was dissolved in a mixture of 30% hydrogen bromide in acetic acid (96 ml) and 48% aqueous hydrobromic acid (24 ml). The reaction was heated to reflux, and stirred at this temperature under nitrogen for 5.5 h. After cooling to room temperature the reaction mixture was poured into water (600 ml) and extracted with diethylether (3×300 ml). The combined organic extracts were washed with water (2×150 ml), brine (100 ml), dried over MgSO₄ and the solvent was removed under reduced pressure. The residue was purified by flash column chromatography on silica gel eluting with a solvent gradient of ethyl acetate:pentane (1:19 changing to 3:37 then 1:9, by volume) and the product was azeotroped with toluene (2×100 ml) to give 3-Methylsulfanyl-phenol (19.3 g) as a crimson oil.

¹H NMR (400 MHz, CDCl₃): δ=7.10-7.20 (1H, t), 6.77-6.84 (1H, d), 6.74 (1H, s), 6.55-6.63 (1H, d), 4.65-4.83 (1H, brs), 2.43 (3H, s) ppm.

Preparation 3

5-Fluoro-2-(3-methylsulfanyl-phenoxy)-nicotinic acid ethyl ester

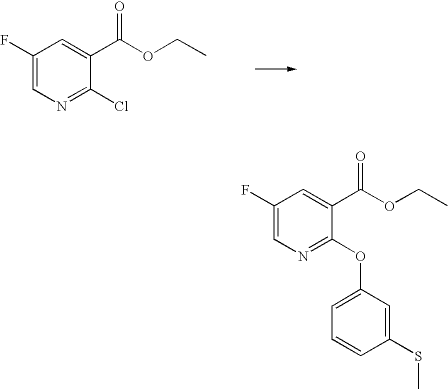

Ethyl 2-chloro-5-fluoro-nicotinate (J. Med. Chem. 1993, 36, 2676, M. Winn et. al.) (5.02 g, 24.7 mmol), 3-methylsulfanylphenol (3.5 g, 25 mmol) and caesium carbonate (8.13 g, 25 mmol) were suspended in dioxan (50 ml) and the reaction was heated to 100° C. and stirred at this temperature under nitrogen for 8 h. The reaction was cooled to room temperature, filtered and the solid washed with ethyl acetate (50 ml). The filtrate was concentrated under reduced pressure, and the residue was purified by flash column chromatography on silica gel eluting with pentane:ethyl acetate (90:10, by volume) and the product was triturated with pentane (10 ml) to give 5-fluoro-2-(3-methylsulfanyl-phenoxy)-nicotinic acid ethyl ester (3.46 g) as a white solid.

¹H NMR (400 MHz, CDCl₃): δ=8.14-8.16 (1H, d), 7.98-8.02 (1H, dd), 7.26-7.31 (1H, t), 7.07-7.11 (1H, d), 7.01 (1H, s), 6.87-6.91 (1H, d), 4.37-4.42 (2H, quart), 2.46 (3H, s), 1.37-1.41 (3H, t) ppm.

LRMS (electrospray): m/z [M+H]⁺ 308, [M+NH₄]⁺ 325, [2M+H]⁺ 615.

Anal. Found C, 58.29; H, 4.54; N, 4.53. C₁₅H₁₄FNO₃S requires C, 58.62; H, 4.54; N, 4.56%.

Preparation 4

5-Fluoro-2-(3-methylsulfanyl-phenoxy)-nicotinic acid

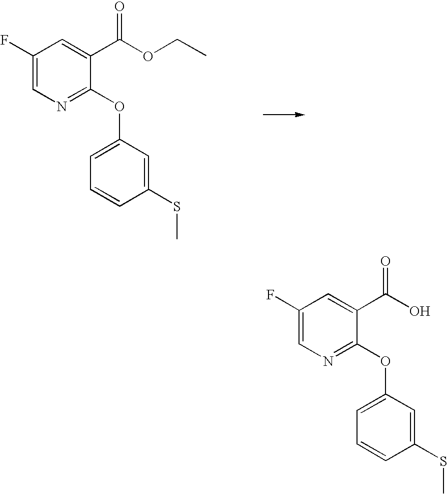

5-Fluoro-2-(3-methylsulfanyl-phenoxy)-nicotinic acid ethyl ester (3.07 g, 10 mmol) was dissolved in tetrahydrofuran (40 ml) and 1M lithium hydroxide solution (25 ml, 25 mmol) was added. The reaction was stirred at room temperature for 18 h and the tetrahydrofuran was removed under reduced pressure. 2N HCl (12.5 ml) was added and the resulting precipitate was isolated by filtration and washed with water (3×50 ml). The wet paste was dissolved in dichloromethane (50 ml), dried over MgSO₄ and the solvent was removed under reduced pressure. The residue was triturated with diisopropylether (15 ml) to give 5-fluoro-2-(3-methylsulfanyl-phenoxy)-nicotinic acid (2.63 g) as a white solid.

¹H NMR (400 MHz, CDCl₃): δ=8.18-8.24 (2H, m), 7.32-7.38 (1H, t), 7.13-7.18 (1H, d), 7.05 (1H, s), 6.89-6.94 (1H, d), 2.47 (3H, s) ppm.

LRMS (thermospray): m/z [M+H]⁺ 280.

Anal. Found C, 55.65; H, 3.57; N, 4.68. C₁₃H₁₀FNO₃S requires C, 55.90; H, 3.61; N, 5.02%.

Preparation 5

tert-butyl 1-acetyl-3-pyrrolidinylcarbamate

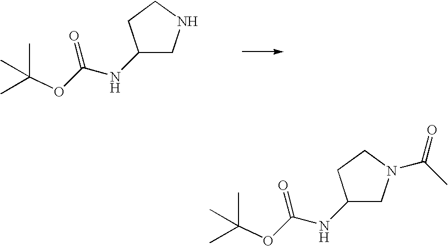

To an ice-cooled solution of racemic tert-butyl 3-pyrrolidinylcarbamate (10.0 g) in methylene chloride (100 ml) and triethylamine (9.0 ml) was added slowly, acetyl chloride (4.5 ml). The mixture was allowed to warm to RT and stirred for 14 h, diluted with methylene chloride (100 ml), washed with dil. hydrochloric acid (100 ml) and brine (100 ml). After drying over MgSO₄, the solvent removed in vacuo to afford the title compound as a pale brown syrup which crystallised upon standing (11.5 g).

¹H NMR (400 MHz, CDCl₃): δ=4.65-4.5 (1H, br m), 4.25-4.1 (1H, br m), 3.7-3.6 (1H, m), 3.55-3.4 (2H, m), 3.3-3.2 (1H, m), 2.2-2.0 (1H, m), 2.0 (3H, app. d), 2.0-1.7 (1H, m), 1.4 (9H, s) ppm.

LRMS (thermospray): m/z [M+H]⁺ 229, [M+NH₄]⁺ 246, [2M+H]⁺ 457.

Anal. Found C, 57.81; H, 8.85; N, 12.28. C₁₁H₂₀N₂O₃ requires C, 57.87; H, 8.83; N, 12.27%.

Preparation 6

tert-butyl 1-propionyl-3-pyrrolidinylcarbamate

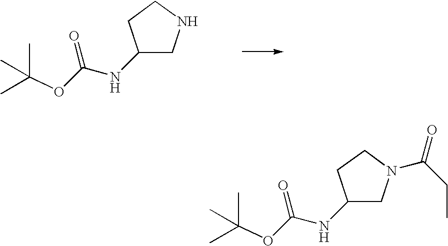

The title compound was prepared using the method of tert-butyl 1-acetyl-3-pyrrolidinylcarbamate using propionyl chloride. Trituration with diethyl ether afforded a white solid (22.25 g, 86%).

¹H NMR (400 MHz, CDCl₃): δ=4.65-4.55 (1H, br m), 4.25-4.1 (1H, br m), 3.7-3.6 (1H, m), 3.56-3.4 (2H, m), 3.35-3.2 (1H, m), 2.2 (2H, q), 2.2-2.0 (1H, m), 1.95-1.85 (0.5H, m), 1.8-1.7 (0.5H, m), 1.4 (9H, s), 1.2 (3H, app dt) ppm.

LRMS (thermospray): m/z [M+H]⁺ 243, [2M+H]⁺ 485.

Anal. Found C, 59.43; H, 9.17; N, 11.50. C₁₂H₂₂N₂O₃ requires C, 59.48; H, 9.15; N, 11.56%.

Preparation 7

tert-butyl 1-isobutyryl-3-pyrrolidinylcarbamate

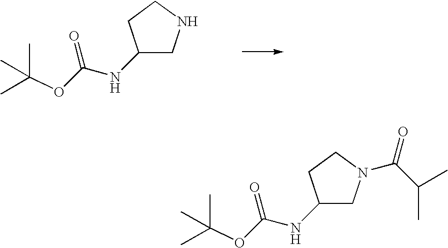

The title compound was prepared using the method of tert-butyl 1-acetyl-3-pyrrolidinylcarbamate using isobutyryl chloride. Trituration with diethyl ether afforded a white solid (24.05 g, 88%).

¹H NMR (400 MHz, CDCl₃): δ=4.55-4.5 (1H, br m), 4.2-4.15 (1H, br m), 3.7-3.6 (1H, m), 3.6-3.4 (2H, m), 3.35-3.3 (1H, m), 2.55-2.5 (1H, m), 2.2-2.0 (1H, m), 1.95-1.85 (0.5H, m), 1.8-1.65 (0.5H, m), 1.4 (9H, s), 1.1 (6H, app dt) ppm.

LRMS (thermospray): m/z [M+H]⁺ 257.

Anal. Found C, 60.90; H, 9.54; N, 10.95. C₁₃H₂₄N₂O₃ requires C, 60.91; H, 9.44; N, 10.93%.

Preparation 8

tert-butyl 1-(3-methylbutanoyl)-3-pyrrolidinylcarbamate

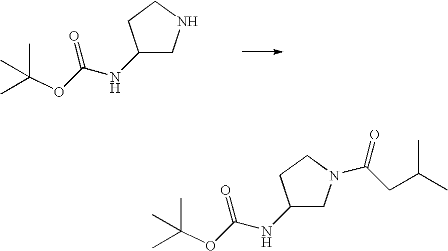

The title compound was prepared using the method of tert-butyl 1-acetyl-3-pyrrolidinylcarbamate using isovaleryl chloride. Trituration with diethyl ether afforded a white solid (24.62 g, 85%).

¹H NMR (400 MHz, CDCl₃): δ=4.65-4.5 (1H, br m), 4.2-4.1 (1H, br m), 3.7-3.6 (1H, m), 3.6-3.4 (2H, m), 3.3-3.2 (1H, m), 2.2-2.05 (4H, m), 1.95-1.85 (0.5H, m), 1.8-1.7 (0.5H, m), 1.4 (9H, s), 0.9 (6H, d) ppm.

LRMS (thermospray): m/z [M+H]⁺ 271.

Anal. Found C, 62.23; H, 9.88; N, 10.37. C₁₄H₂₆N₂O₃ requires C, 62.19; H, 9.69; N, 10.36%.

Preparation 9

1-acetyl-3-pyrrolidinamine

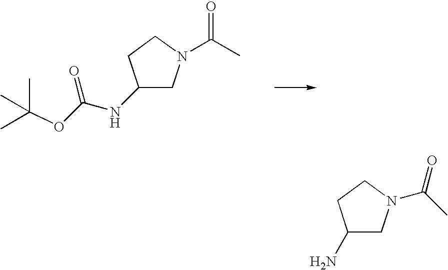

To a stirred solution of tert-butyl 1-acetyl-3-pyrrolidinylcarbamate (20.6 g, 90 mmol) in methylene chloride (50 ml) at RT was added trifluoroacetic acid (25 ml). After 14 h, the reaction mixture was concentrated in vacuo and azeotroped with toluene twice to yield a pale yellow oil. This was taken up in water (100 ml), basified with solid Na₂CO₃ and passed through a Dowex™ 50W8 ion exchange column eluting with water (1 l), then 5% aq. ammonium hydroxide (2 l). The appropriate fractions were freeze-dried and azeotroped with methylene chloride to afford a yellow oil (11.10 g, 86.7 mmol)

¹H NMR (400 MHz, CDCl₃): δ=3.55-3.5 (3H, m), 3.45-3.4 (1H, m), 3.15 (0.5H, q), 3.05 (0.5H, q), 2.15-2.0 (1H, m), 2.0 (3H, s), 1.8-1.6 (1H, m), 1.3-1.1 (2H, br s) ppm.

LRMS (thermospray): m/z [2M+H]⁺ 257.

Anal. Found C, 54.57; H, 9.41; N, 20.95. C₆H₁₂N₂O.0.2H₂O requires C, 54.69; H, 9.48; N, 21.26%.

Preparation 10

1-propionyl-3-pyrrolidinamine

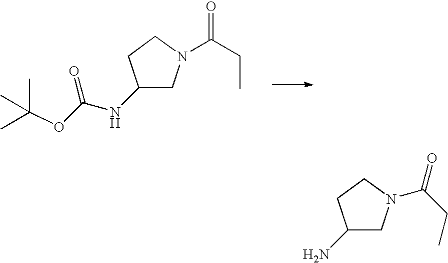

The title compound was prepared following the method of 1-acetyl-3-pyrrolidinamine as a colourless oil (9.0 g, 69%).

¹H NMR (400 MHz, CDCl₃): δ=3.65-3.5 (3H, m), 3.45-3.35 (1H, m), 3.15 (0.5H, q), 3.05 (0.5H, q), 2.2 (2H, q), 2.1-2.0 (1H, m), 1.75-1.65 (0.5H, m), 1.6-1.55 (0.5H, m), 1.4 (2H, br s), 1.1 (3H, t) ppm.

LRMS (thermospray): m/z [2M+H]⁺ 285.

Anal. Found C, 57.58; H, 10.12; N, 19.13. C₇H₁₄N₂O.0.2H₂O requires C, 57.66; H, 9.95; N, 19.21%.

Preparation 11

1-isobutyryl-3-pyrrolidinamine

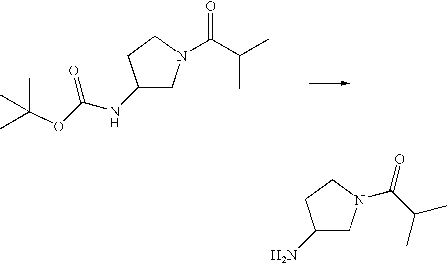

The title compound was prepared following the method of 1-acetyl-3-pyrrolidinamine as a brown oil (11.64 g, 89%).

¹H NMR (400 MHz, CDCl₃): δ=3.65-3.5 (3H, m), 3.5-3.4 (1H, m), 3.2-3.15 (1H, m), 2.65-2.5 (1H, m), 2.1-1.95 (1H, m), 1.75-1.65 (0.5H, m), 1.6-1.55 (0.5H, m), 1.2 (1H, br s), 1.1 (6H, dd) ppm.

LRMS (thermospray): m/z [M+H]⁺ 157, [2M+H]⁺ 313.

Anal. Found C, 59.90; H, 10.36; N, 17.33. C₈H₁₅N₂O.0.2H₂O requires C, 60.12; H, 10.34; N, 17.53%.

Preparation 12

1-(3-methylbutanoyl)-3-pyrrolidinamine

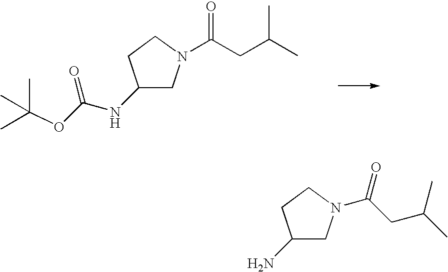

The title compound was prepared following the method of 1-acetyl-3-pyrrolidinamine as a brown oil (12.44 g, 84%).

¹H NMR (400 MHz, CDCl₃): δ=3.65-3.5 (3H, m), 3.5-3.4 (1H, m), 3.2-3.1 (0.5H, m), 3.1-3.0 (0.5H, m), 2.2-1.9 (4H, m), 1.75-1.65 (0.5H, m), 1.65-1.55 (0.5H, m), 1.2 (2H, br s), 0.9 (6H, d) ppm.

LRMS (thermospray): m/z [M+H]⁺ 171, [2M+H]⁺ 341.

Anal. Found C, 61.65; H, 10.64; N, 15.95. C₉H₁₇N₂O.0.3H₂O requires C, 61.54; H, 10.67; N, 15.95%.

Preparation 13

tert-butyl 4-{[(2-chloro-5-fluoro-3-pyridinyl)carbonyl]amino}-1-piperidinecarboxylate

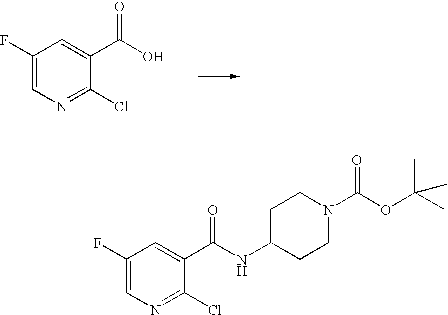

Carbonyl diimidazole (2.8 g, 17.3 mmol) was added to a solution of 2-chloro-5-fluoronicotinic acid (3.0 g, 17.1 mmol) in dimethylformamide (30 ml). The mixture was stirred for 1 h, then tert-butyl 4-aminopiperdinylcarbamate (3.8 g, 19.0 mmol) was added. The reaction was stirred under nitrogen at room temperature for 18 h, the solvent removed under reduced pressure and the residue was partitioned between 10% aqueous citric acid solution (200 m) and ethyl acetate (200 ml). The organic phase was washed with water and saturated sodium bicaronate solution, dried over MgSO₄ and the solvent was removed under reduced pressure to give the title compound (4.4 g) as a white solid.

¹H NMR (400 MHz, CDCl₃): δ=8.33-8.36 (1H, m), 7.84-7.88 (1H, m), 6.40-6.48 (1H, m), 3.92-4.20 (3H, m), 2.90-3.05 (2H, t), 1.97-2.07 (2H, m), 1.40-1.55 (11H, m) ppm.

LRMS (electrospray): m/z [M+Na]⁺ 380

Anal. Found C, 53.91; H, 6.01; N, 11.70. C₁₆H₂₁ClFN₃O₃ requires C, 53.71; H, 5.92; N, 11.74%.

Preparation 14

2-[3-(methylsulfanyl)benzyl]-N-(4-piperidinyl)nicotinamide

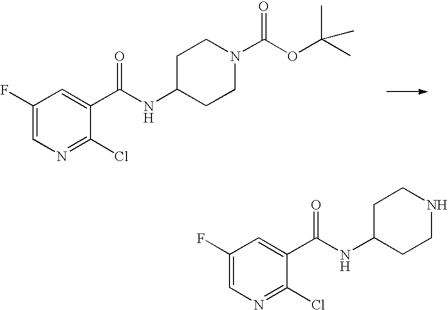

Methane sulphonic acid (145 μl, 2.24 mmol) was added to a solution of tert-butyl 4-[({2-[3-(methylsulfanyl)benzyl]-3-pyridinyl}carbonyl)amino]-1-piperidinecarboxylate (200 mg, 0.56 mmol) in dichloromethane (5 ml) at 0° C. The mixture was warmed to ambient temperature and stirred for 2 h. The solvent removed under reduced pressure to give 2-[3-(methylsulfanyl)benzyl]-N-(4-piperidinyl)nicotinamide which was used in the next step without further purification

¹H NMR (400 MHz, CDCl₃): δ=8.35-8.40 (1H, m), 7.84-7.90 (1H, m), 4.10-4.20 (1H, m), 3.08-3.20 (2H, t), 2.17-2.28 (2H, m), 1.78-1.92 (2H, m) ppm.

LRMS (electrospray): m/z [M+H]⁺ 258

Preparation 15

N-(1-acetyl-4-piperidinyl)-2-[3-(methylsulfanyl)benzyl]nicotinamide

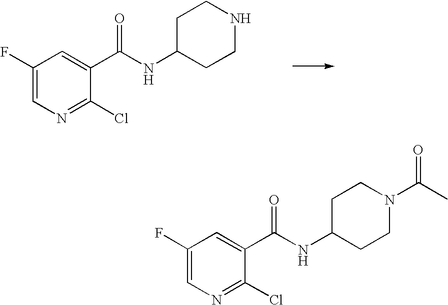

To a solution of 2-[3-(methylsulfanyl)benzyl]-N-(4-piperidinyl)nicotinamide, triethylamine (623 μl, 4.5 mmol) and 4-N,N-dimethylaminopyridine (5 mg) in dichloromethane (5 ml) was added acetyl chloride (60 μl, 0.84 mmol) at 0° C. The reaction mixture was warmed to ambient temperature then stirred for 2 h. The solvent was removed under reduced pressure and the residue was purified by flash column chromatography using dichloromethane:methanol:0.880 ammonia (98:2:0.2 to 91:9:0.9) as eluant to give N-(1-acetyl-4-piperidinyl)-2-[3-(methylsulfanyl)benzyl]nicotinamide (147 mg).

¹H NMR (400 MHz, CDCl₃): δ=8.30-8.35 (1H, m), 7.85-7.90 (1H, m), 6.45-6.55 (1H, m), 4.48-4.56 (1H, m), 4.16-4.26 (1H, m), 3.77-3.85 (1H, m), 3.20-3.30 (1H, t), 2.80-2.90 (1H, t), 2.03-2.25 (4H, m+s), 1.42-1.55 (2H, m) ppm.

LRMS (electrospray): m/z [M+Na]⁺ 322

Preparation 17

2-fluoro-4-(methylsulfanyl)phenol

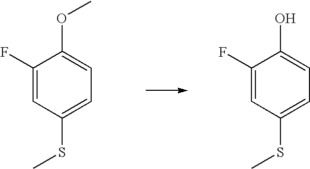

A stirred solution of 2-fluoro-1-methoxy-4-(methylsulfanyl)benzene (2.0 g, 11.6 mmol) in methylene chloride (80 ml) at 0° C. was treated slowly with boron tribromide (1M in CH₂Cl₂, 23 ml, 23 mmol), after which it was allowed to warm to RT. A further portion of BBr₃ was added (1M, 10 ml, 10 mmol) and the reaction mixture allowed to stand at RT overnight. After cooling to 0° C., the reaction mixture was cautiously quenched with a solution of diethanolamine (10 ml) in methylene chloride (40 ml), partioned between methylene chloride and 2N HCl, and the organic phase separated, and washed with water and brine. After drying over Na₂SO₄, the organics were concentrated in vacuo. The title compound was isolated following purification by flash column chromatography on silica gel eluting with a solvent gradient cyclohexane to 20% ethanol:cyclohexane to afford a brown oil (1.63 g, 10.3 mmol).

¹H NMR (400 MHz, CDCl₃): δ=7.1-6.9 (3H, m), 5.1 (1H, s), 2.4 (3H, s) ppm.

Preparation 18

2-[(cyclopropylmethyl)sulfanyl]phenol

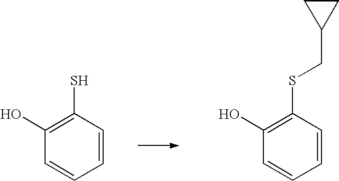

A solution of 2-hydroxythiophenol (25.65 g, 0.2 mol) in ethanol (30 ml) was added to a stirred solution of sodium hydroxide (8 g, 0.2 mol) in ethanol (100 ml) over 10 mins. After stirring for 20 mins at RT, cyclopropylmethylbromide (27.5 g, 0.2 mol) was added over 5 mins and the mixture heated to reflux for 15 mins. After cooling, the suspension was filtered to remove a white solid and the filtrate concentrated to a brown oil which was partioned between diethylether (100 ml) and water (100 ml) with aq HCl (5.6M, 10 ml). The ethereal phase was removed, dried over MgSO4 and condensed to a brown oil which distilled. The fraction boiling at 132-134° C. (13-14 mmHg) was collected to afford the title compound as a pale yellow oil (22.39 g, 0.12 mol).

Anal. Found C, 66.51; H, 6.75. C₁₀H₁₂OS requires C, 66.63; H, 6.71.

IR (film) 3380 cm⁻¹ (OH)

Preparation 19

(2-amino-3-pyridinyl)methanol

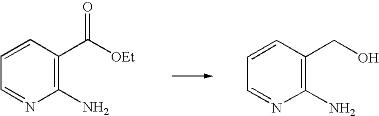

Lithium aluminium hydride (1M in THF, 43 ml, 43 mmol) in dry diethyl ether (65 ml) was treated with a slurry of ethyl 2-aminonicotinate (6.53 g, 39.3 mmol) in ether (50 ml) dropwise over ½ h maintaining T<15° C. The resultant heterogeneous mixture was stirred at RT for 1 h, cooled in ice, and quenched with ethyl acetate (caution: dropwise addition), then water. After removal of the solvent in vacuo, the solid was extracted with hot acetone to afford a mobile oil after evaporation. Crystallisation from diisopropyl ether gave the title compound as fine needles (3.75 g, 30.2 mmol).

¹H NMR (300 MHz, CDCl₃): δ=8.0 (1H, dd), 7.25 (1H, dd), 6.5 (1H, dt), 5.0 (2H, br s), 4.6 (2H, s), 2.0 (1H, br s) ppm.

LRMS (thermospray): m/z [M+H]⁺ 125.

Preparation 20

3-ethoxy-2-pyridinamine

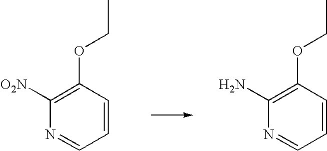

Hydrogenation of 3-ethoxy-2-nitropyridine (5.0 g, 30 mmol) in ethanol (150 ml) was effected over PtO₂ in a Parr bottle at 40° C. over hydrogen (50 psi) for 3 h. The catalyst was removed by filtration, and the solvent removed in vacuo to give a brown gum which was extracted with boiling pentane to afford the title compound as a white solid (3.1 g, 22 mmol).

mp 84° C.

### EXAMPLES

### Example 1

### N-(1-Acetyl-pyrrolidine-3-yl)-5-fluoro-2-(3-methylsulfanyl-phenoxy)-nicotinamide

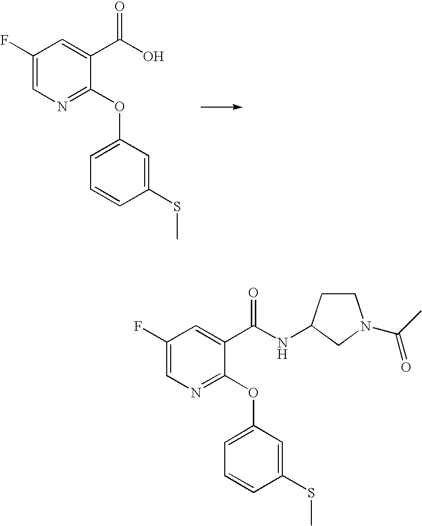

Carbonyldiimidazole (64 mg, 0.393 mmol) was added to a suspension of 5-fluoro-2-(3-methylsulfanyl-phenoxy)-nicotinic acid (100 mg, 0.358 mmol) in dichloromethane (1.5 ml) under nitrogen at room temperature. The resulting solution was stirred for 15 min after which a solution of 1-(3-amino-pyrrolidin-1-yl)-ethanone (51 mg, 0.393 mmol) and 4-dimethylaminopyridine (2 mg, 0.02 mmol) in dichloromethane (1.5 ml) was added. The reaction was stirred at room temperature for 4 h then quenched with sat. ammonium chloride solution (0.5 ml) and diluted with dichloromethane (5 ml). The mixture was then passed through a phase separation cartridge, and the solvent was removed from the organic phase under reduced pressure. The residue was purified by flash column chromatography on silica gel eluting with a solvent gradient of dichloromethane:methanol (99:1 changing to 98:2, by volume) to give N-(1-Acetyl-pyrrolidine-3-yl)-5-fluoro-2-(3-methylsulfanyl-phenoxy)-nicotinamide (50 mg) as an off-white solid which was a mixture of rotamers.

¹H NMR (400 MHz, CDCl₃): δ=8.28-8.33 (1H, m), 8.03-8.06 (1H, m), 7.97-8.01 (0.5H, d), 7.90-7.95 (0.5H, d), 7.30-7.37 (1H, m), 7.12-7.16 (1H, d), 6.95-6.99 (1H, m), 6.81-6.86 (1H, t), 4.62-4.72 (1H, m), 3.76-3.88 (1H, 2xm), 3.55-3.61 (1H, t), 3.48-3.55 (1H, t), 3.37-3.47 (1H, m), 2.47 (3H, s), 2.32-2.41 (0.5H, m), 2.21-2.32 (0.5H, m), 1.97-2.08 (3.5H, 2xs+m), 1.83-1.95 (0.5H, m) ppm.

LRMS (electrospray): m/z [M+Na]⁺ 412, [M−H]⁺ 388

### Example 2

### 5-Fluoro-2-(3-methylsulfanyl-phenoxy)-N-(1-propionyl-pyrrolidin-3-yl)-nicotinamide

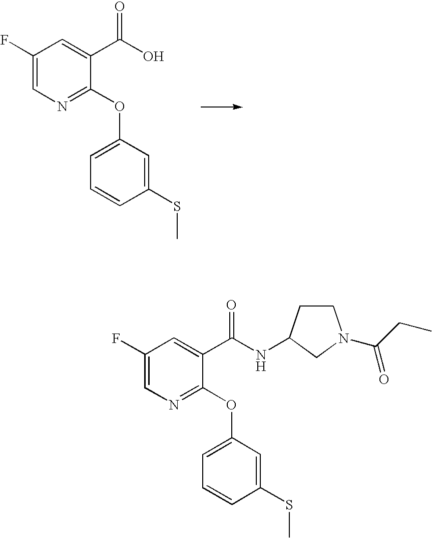

Carbonyldiimidazole (64 mg, 0.393 mmol) was added to a suspension of 5-fluoro-2-(3-methylsulfanyl-phenoxy)-nicotinic acid (100 mg, 0.358 mmol) in dichloromethane (1.5 ml) under nitrogen at room temperature. The resulting solution was stirred for 15 min after which a solution of 1-(3-amino-pyrrolidin-1-yl)-propionone (56 mg, 0.393 mmol) and 4-dimethylaminopyridine (2 mg, 0.02 mmol) in dichloromethane (1.5 ml) was added. The reaction was stirred at room temperature for 4 h then quenched with sat. ammonium chloride solution (0.5 ml) and diluted with dichloromethane (5 ml). The mixture was then passed through a phase separation cartridge, and the solvent was removed from the organic phase under reduced pressure. The residue was purified by flash column chromatography on silica gel eluting with a solvent gradient of dichloromethane:methanol (99:1 changing to 98:2, by volume) to give N-(1-Acetyl-pyrrolidine-3-yl)-5-fluoro-2-(3-methylsulfanyl-phenoxy)-nicotinamide (101 mg) as an off-white solid which was a mixture of rotamers.

¹H NMR (400 MHz, CDCl₃): δ=8.29-8.32 (1H, dd), 8.03-8.05 (1H, m), 7.95-8.00 (0.5H, d), 7.78-7.93 (0.5H, d), 7.29-7.36 (1H, m), 7.14-7.18 (1H, d), 6.96-6.99 (1H, m), 6.81-6.86 (1H, t), 4.63-4.71 (1H, m), 3.77-3.85 (1H, m), 3.56-3.61 (1H, t), 3.44-3.53 (1.5H, m), 3.37-3.43 (0.5H, m), 2.47 (3H, s), 2.30-2.41 (0.5H, m), 2.16-2.29 (2.5H, m), 2.00-2.12 (0.5H, m), 1.85-1.95 (0.5H, m), 1.06-1.11 (3H, m) ppm.

LRMS (electrospray): m/z [M+Na]⁺ 426, [M−H]⁺ 402

### Example 3

### 5-Fluoro-N-(1-isobutyryl-pyrrolidin-3-yl)-2-(3-methylsulfanyl-phenoxy)-nicotinamide

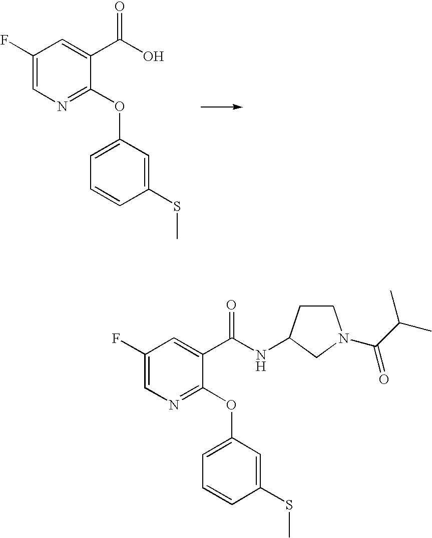

Carbonyidiimidazole (64 mg, 0.393 mmol) was added to a suspension of 5-fluoro-2-(3-methylsulfanyl-phenoxy)-nicotinic acid (100 mg, 0.358 mmol) in dichloromethane (1.5 ml) under nitrogen at room temperature. The resulting solution was stirred for 15 min after which a solution of 1-(3-amino-pyrrolidin-1-yl)-2-methyl-propan-1-one (62 mg, 0.393 mmol) and 4-dimethylaminopyridine (2 mg, 0.02 mmol) in dichloromethane (1.5 ml) was added. The reaction was stirred at room temperature for 4 h then quenched with sat. ammonium chloride solution (0.5 ml) and diluted with dichloromethane (5 ml). The mixture was then passed through a phase separation cartridge, and the solvent was removed from the organic phase under reduced pressure. The residue was purified by flash column chromatography on silica gel eluting with a solvent gradient of dichloromethane:methanol (99:1 changing to 98:2, by volume) to give N-(1-Acetyl-pyrrolidine-3-yl)-5-fluoro-2-(3-methylsulfanyl-phenoxy)-nicotinamide (110 mg ) as an off-white solid which was a mixture of rotamers.

¹H NMR (400 MHz, CDCl₃): δ=8.29-8.33 (1H, dd), 8.04-8.07 (1H, t), 7.94-8.00 (0.5H, d), 7.90-7.94 (0.5H, d), 7.28-7.35 (1H, m), 7.12-7.16 (1H, d), 6.94-6.99 (1H, m), 6.80-6.86 (1H, t), 4.63-4.72 (1H, m), 3.85-3.92 (0.5H, m), 3.75-3.81 (0.5H, m), 3.55-3.61 (2H, t), 3.43-3.51 (1H, m), 2.50-2.61 (1H, m), 2.45 (3H, s), 2.30-2.42 (0.5H, m), 2.20-2.30 (0.5H, m), 2.01-2.10 (0.5H, m), 1.84-1.96 (0.5H, m), 1.06-1.12 (3H, m), 0.98-1.06 (3H, 2xd) ppm.

LRMS (electrospray): m/z [M+Na]⁺ 440, [M−H]⁺ 416

### Example 4

### 5-Fluoro-N-[1-(3-methyl-butyryl)-pyrrolidin-3-yl]-2-(3-methylsulfanyl-phenoxy)-nicotinamide

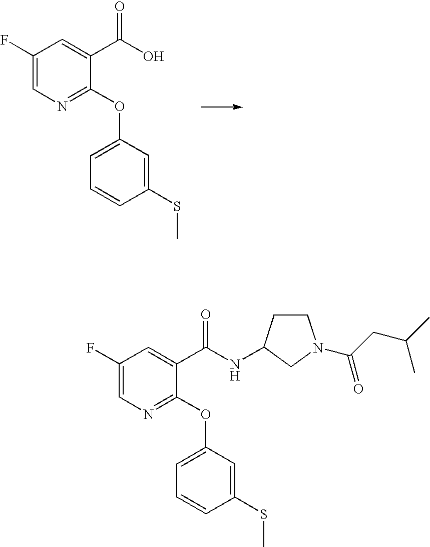

Carbonyldiimidazole (64 mg, 0.393 mmol) was added to a suspension of 5-fluoro-2-(3-methylsulfanyl-phenoxy)-nicotinic acid (100 mg, 0.358 mmol) in dichloromethane (1.5 ml) under nitrogen at room temperature. The resulting solution was stirred for 15 min after which a solution of 1-(3-amino-pyrrolidin-1-yl)-3-methyl-butan-1-one (67 mg, 0.393 mmol) and 4-dimethylaminopyridine (2 mg, 0.02 mmol) in dichloromethane (1.5 ml) was added. The reaction was stirred at room temperature for 4 h then quenched with sat. ammonium chloride solution (0.5 ml) and diluted with dichloromethane (5 ml). The mixture was then passed through a phase separation cartridge, and the solvent was removed from the organic phase under reduced pressure. The residue was purified by flash column chromatography on silica gel eluting with a solvent gradient of dichloromethane:methanol (99:1 changing to 98:2, by volume) to give N-(1-Acetyl-pyrrolidine-3-yl)-5-fluoro-2-(3-methylsulfanyl-phenoxy)-nicotinamide (104 mg) as an off-white solid which was a mixture of rotamers.

¹H NMR (400 MHz, CDCl₃): δ=8.30-8.33 (1H, dd), 8.02-8.05 (1H, m), 7.96-8.00 (0.5H, d), 7.90-7.95 (0.5H, d), 7.28-7.36 (1H, m), 7.10-7.16 (1H, d), 6.95-6.99 (1H, m), 6.80-6.85 (1H, t), 4.62-4.70 (1H, m), 3.82-3.87 (0.5H, m), 3.77-3.82 (0.5H, m), 3.56-3.61 (2H, t), 3.38-3.55 (2H, 2xm), 2.44 (3H, s), 2.30-2.40 (0.5H, m), 2.19-2.29 (0.5H, m), 2.01-2.18 (2.5H, m), 1.85-1.96 (0.5H, m), 0.83-0.96 (6H, m) ppm.

LRMS (electrospray): m/z [M+Na]⁺ 454, [M−H]⁺ 430

### Example 5

### 5-Fluoro-2-(3-methylsulfanyl-phenoxy)-N-piperidin-4-yl-nicotinamide

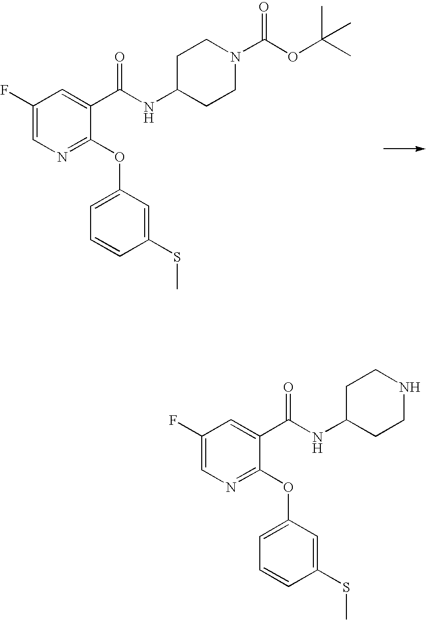

Trifluoroacetic acid (25 ml) was added to a solution of 4-{[5-fluoro-2-(3-methylsulfanyl-phenoxy)-pyridin-3-carbonyl]-amino}-piperidine-1-carboxylic acid tert-butyl ester (2.3 g, 4.99 mmol) and in dichloromethane (25 ml) and the reaction was stirred under nitrogen at room temperature for 2 h. The solvent was removed under reduced pressure and the residue was partitioned between ethyl acetate (100 ml) and sat. sodium bicarbonate solution (100 ml). The organic phase was removed, washed with water (50 ml), brine (50 ml), dried over MgSO₄ and the solvent was removed under reduced pressure. The residue was purified by flash column chromatography on silica gel eluting with a solvent gradient of dichloromethane:methanol:concentrated aqueous ammonia (95:5:0.5 changing to 85:15:1.5, by volume) to give 5-Fluoro-2-(3-methylsulfanyl-phenoxy)-N-piperidin-4-yl-nicotinamide (110 mg) as an off-white solid.

¹H NMR (400 MHz, CD₃OD): δ=8.08-8.10 (1H, d), 7.97-8.00 (1H, m), 7.27-7.32 (1H, t), 7.11-7.15 (1H, d), 7.04 (1H, s), 6.84-6.88 (1H, d), 4.03-4.13 (1H, m), 3.20-3.30 (2H, m, partially masked by solvent), 2.88-2.98 (2H, m), 2.45 (3H, s), 2.03-2.13 (2H, m), 1.57-1.73 (2H, m) ppm.

LRMS (electrospray): m/z [M+H]⁺ 362.

Anal. Found C, 54.18; H, 5.18; N, 10.26. C₁₈H₂₀FN₃O₂S. 0.6 mol CH₂Cl₂ requires C, 54.17; H, 5.18; N, 10.19%.

### Example 6

### 5-Fluoro-N-(1-methyl-piperidin-4-yl)-2-(3-methylsulfanyl-phenoxy)-nicotinamide

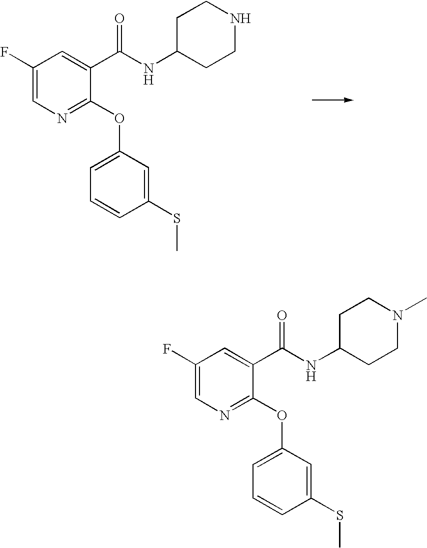

37% Aqueous formaldehyde (33 μl, 0.404 mmol) was added to a solution of 5-fluoro-2-(3-methylsulfanyl-phenoxy)-N-piperidin-4-yl-nicotinamide (107 mg, 0.269 mmol) in dichloromethane (4 ml) under nitrogen at room temperature and the reaction was stirred for 1 h. Sodium triacatoxyborohydride (125 mg, 0.538 mmol) was then added and the reaction was stirred at room temperature for 3 h. The reaction was diluted with dichloromethane (5 ml), quenched with sat. sodium bicarbonate solution (1 ml) and further diluted with water (3 ml). The organic phase was removed, concentrated under reduced pressure and the residue was purified by flash column chromatography on silica gel eluting with dichloromethane:methanol:concentrated aqueous ammonia (98:2:0.2 changing to 90:10:1, by volume) to give 5-fluoro-N-(1-methyl-piperidin-4-yl)-2-(3-methylsulfanyl-phenoxy)-nicotinamide (85 mg) as an off-white solid.

¹H NMR (400 MHz, CD₃OD): δ=8.06-8.08 (1H, d), 7.97-8.00 (1H, m), 7.28-7.33 (1H, t), 7.09-7.13 (1H, d), 7.04 (1H, s), 6.87-6.91 (1H, d), 3.86-3.96 (1H, m), 2.75-2.83 (2H, d), 2.45 (3H, s), 2.18-2.27 (5H, s+m), 1.93-2.00 (2H, m), 1.58-1.66 (2H, m) ppm.

LRMS (electrospray): m/z [M+H]⁺ 376, [M+Na]⁺ 398 , [M−H]⁺ 374.

Anal. Found C, 59.27; H, 5.83; N, 9.91. C₁₉H₂₂FN₃O₂S. 0.1 mol H₂O. 0.15 mol CH₂Cl₂ requires C, 58.98; H, 5.82; N, 10.77%.

### Example 7

### 5-Fluoro-N-(1-ethyl-piperidin-4-yl)-2-(3-methylsulfanyl-phenoxy)-nicotinamide

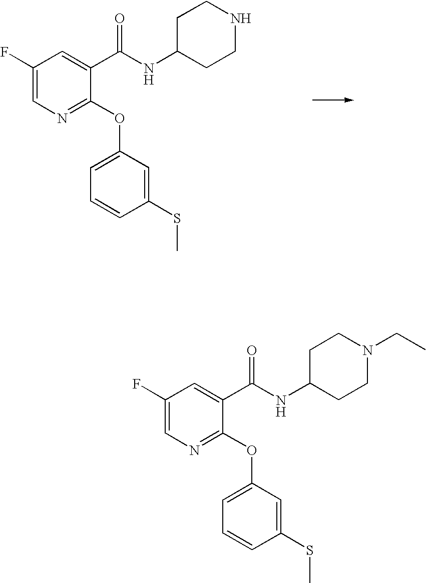

Acetaldehyde (25 μl, 0.404 mmol) was added to a solution of 5-fluoro-2-(3-methylsulfanyl-phenoxy)-N-piperidin-4-yl-nicotinamide (107 mg, 0.269 mmol) in dichloromethane (4 ml) under nitrogen at room temperature and the reaction was stirred for 1 h. Sodium triacatoxyborohydride (125 mg, 0.538 mmol) was then added and the reaction was stirred at room temperature for 3 h. The reaction was diluted with dichloromethane (5 ml), quenched with sat. sodium bicarbonate solution (1 ml) and further diluted with water (3 ml). The organic phase was removed, concentrated under reduced pressure and the residue was purified by flash column chromatography on silica gel eluting with dichloromethane:methanol:concentrated aqueous ammonia (98:2:0.2 changing to 90:10:1, by volume) to give 5-fluoro-N-(1-ethyl-piperidin-4-yl)-2-(3-methylsulfanyl-phenoxy)-nicotinamide (20 mg) as an off-white solid.

¹H NMR (400 MHz, CD₃OD): δ=8.07-8.09 (1H, d), 7.97-8.00 (1H, m), 7.27-7.33 (1H, t), 7.09-7.13 (1H, d), 7.04 (1H, s), 6.86-6.90 (1H, d), 3.90-3.96 (1H, m), 2.83-2.92 (2H, m), 2.40-2.47 (5H, s+quart), 2.17-2.24 (2H, m), 1.95-2.02 (2H, m), 1.58-1.68 (2H, m), 1.05-1.12 (3H, t) ppm.

LRMS (electrospray): m/z [M+H]⁺ 391, [M+Na]⁺ 412 , [M−H]⁺ 389.

HRMS: Found [M+H]⁺ 390.4612. C₂₀H₂₅FN₃O₂S requires 390.1646.

### Example 8

### 5-Fluoro-N-[1-(2-hydroxy-ethyl)-piperidin-4-yl]-2-(3-methylsulfanyl-phenoxy)-nicotinamide

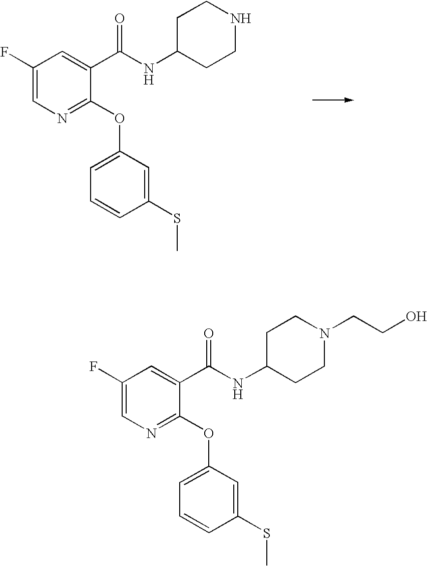

Glyceraldehyde dimer (27 mg, 0.202 mmol) was added to a solution of 5-fluoro-2-(3-methylsulfanyl-phenoxy)-N-piperidin-4-yl-nicotinamide (107 mg, 0.269 mmol) in dichloromethane (4 ml) under nitrogen at room temperature and the reaction was stirred for 1 h. Sodium triacatoxyborohydride (125 mg, 0.538 mmol) was then added and the reaction was stirred at room temperature for 3 h. The reaction was diluted with dichloromethane (5 ml), quenched with sat. sodium bicarbonate solution (1 ml) and further diluted with water (3 ml). The organic phase was removed, concentrated under reduced pressure and the residue was purified by flash column chromatography on silica gel eluting with dichloromethane:methanol:concentrated aqueous ammonia (98:2:0.2 changing to 90:10:1, by volume) to give 5-Fluoro-N-[1-(2-hydroxy-ethyl)-piperidin-4-yl]-2-(3-methylsulfanyl-phenoxy)-nicotinamide (83 mg) as an off-white solid.

¹H NMR (400 MHz, CD₃OD): δ=8.07-8.09 (1H, d), 7.96-8.00 (1H, dd), 7.23-7.35 (1H, t), 7.10-7.14 (1H, d), 7.04 (1H, s), 6.85-6.91 (1H, d), 3.87-3.97 (1H, m), 3.62-3.66 (2H, t), 2.87-2.93 (2H, m), 2.48-2.55 (2H, t), 2.45 (3H, s), 2.24-2.32 (2H, m), 1.93-2.00 (2H, d), 1.77-1.89 (2H, d) ppm.

LRMS (electrospray): m/z [M+H]⁺ 406, [M+Na]⁺ 428 , [M−H]⁺ 404.

Anal. Found C, 57.13; H, 5.74; N, 9.82. C₂₀H₂₄FN₃O₃S. 0.7 mol H₂O requires C, 57.46; H, 6.12; N, 10.05%.

### Example 9

### N-(1-Acetyl-piperidin-4-yl)-5-fluoro-2-(3-methylsulfanyl-phenoxy)-nicotinamide

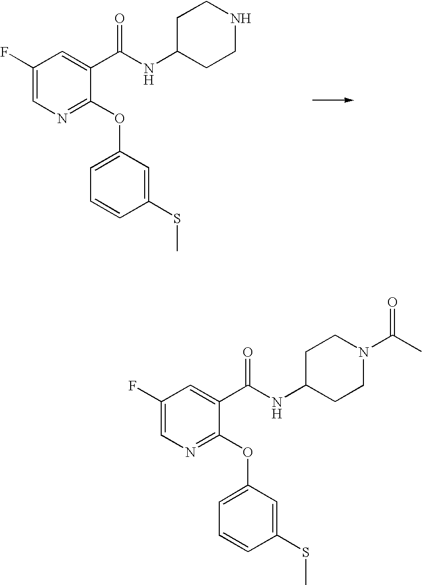

5-Fluoro-2-(3-methylsulfanyl-phenoxy)-N-piperidin-4-yl-nicotinamide (107 mg, 0.269 mmol) and N,N-dimethylaminopyridine (5 mg) were suspended in dichloromethane (4 ml) under nitrogen at 0° C. and triethylamine (83□l, 0.538 mmol) was added followed by acetyl chloride (23 μl, 0.296 mmol). The reaction was allowed to warm to room temperature for 2 h and was quenched with sat. sodium bicarbonate solution (1 ml). After diluting with water (4 ml) the aqueous phase was extractred with dichloromethane (10 ml) and the organic phase was removed and concentrated under reduced pressure. The residue was purified by flash column chromatography on silica gel eluting with dichloromethane:methanol:concentrated aqueous ammonia (99:1:0.1 changing to 92:8:0.8, by volume) to give N-(1-Acetyl-piperidin-4-yl)-5-fluoro-2-(3-methylsulfanyl-phenoxy)-nicotinamide (82 mg) as a white foam.

¹H NMR (400 MHz, CD₃OD): δ=8.08-8.10 (1H, d), 7.97-8.00 (1H, m), 7.28-7.34 (1H, t), 7.10-7.14 (1H, d), 7.04 (1H, s), 6.86-6.90 (1H, d), 4.30-4.38 (1H, d), 4.09-4.19 (1H, m), 3.80-3.88 (1H, d), 3.21-3.30 (1H, m, partially masked by solvent), 2.86-2.94 (1H, t), 2.45 (3H, s), 2.09 (3H, s), 1.94-2.08 (2H, m), 1.40-1.60 (2H, m) ppm.

LRMS (electrospray): m/z [M+Na]⁺ 426, [2M+Na]⁺ 829, [M−H]⁺ 402.

Anal. Found C, 58.41; H, 5.49; N, 9.84. C₂₀H₂₂FN₃O₃S. 0.55 mol H₂O requires C, 58.11; H, 5.63; N, 10.16%.

### Example 10

### 5-Fluoro-2-(3-methylsulfanyl-phenoxy)-N-[1-(3-pyridin-2-yl-propionyl)-piperidin-4-yl]-nicotinamide

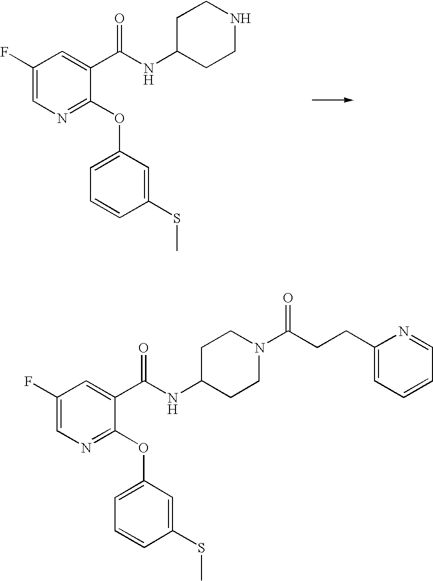

1-(3-Dimethylaminopropyl)-3-ethylcarbodiimide hydrochloride (91 mg, 0.479 mmol), 1-hydroxybenzotriazole (47 mg, 0.351 mmol) and 3-pyridinepropionic acid (72 mg, 0.479 mmol) were added to a solution of 5-fluoro-2-(3-methylsulfanyl-phenoxy)-N-piperidin-4-yl-nicotinamide (115 mg, 0.319 mmol) in dichloromethane (5 ml) under nitrogen under room temperature and N-methylmorpholine (70 μl, 0.638 mmol) was added. The reaction was stirred at room temperature for 18 h. The reaction mixture was quenched with sat. ammonium chloride (1 ml) and diluted with dichloromethane (5 ml) and water (1 ml). The organic phase was collected via a hydrophobic separation cartridge, concentrated under reduced pressure and the residue was purified by flash column chromatography on silica gel eluting with a solvent gradient of dichloromethane:methanol:concentrated aqueous ammonia (99:1:0.1 changing to 95:5:0.5, by volume) to give 5-fluoro-2-(3-methylsulfanyl-phenoxy)-N-[1-(3-pyridin-2-yl-propionyl)-piperidin-4-yl]-nicotinamide (167 mg) as a yellow gum.

¹H NMR (400 MHz, CD₃OD): δ=8.42 (1H, s), 8.27-8.30 (1H, d), 8.08-8.10 (1H, d), 7.97-8.00 (1H, m), 7.72-7.76 (1H, d), 7.29-7.34 (2H, m), 7.08-7.13 (1H, d), 7.05 (1H, s), 6.86-6.90 (1H, m), 4.30-4.38 (1H, d), 4.05-4.15 (1H, m), 3.82-3.90 (1H, d), 3.16-3.24 (1H, t), 2.84-2.96 (3H, t+m), 2.70-2.78 (2H, m), 2.45 (3H, s), 1.91-1.99 (2H, d), 1.30-1.43 (2H, m) ppm.

LRMS (electrospray): m/z [M+Na]⁺ 517, [M−H]⁺ 493.

Anal. Found C, 61.95; H, 5.52; N, 10.91. C₂₆H₂₇FN₄O₃S. 0.5 mol H₂O requires C, 62.01; H, 5.60; N, 11.13%.

### Example 11

### 5-Fluoro-2-(3-methylsulfanyl-phenoxy)-N-(1-propionyl-piperidin-4-yl)-nicotinamide

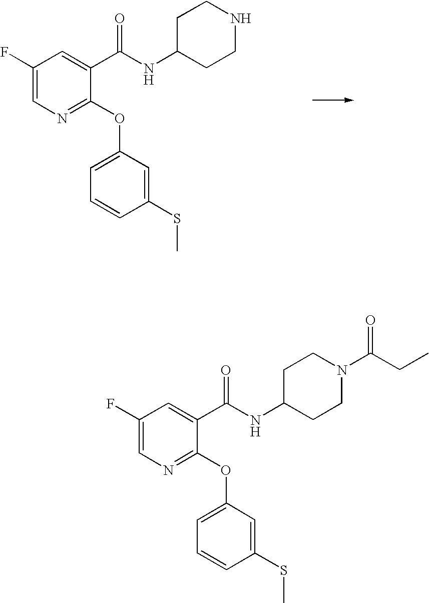

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by propionyl chloride (39 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120 μl, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-(1-propionyl-piperidin-4-yl)-nicotinamide (90 mg) as an off-white solid.

¹H NMR (400 MHz, CD₃OD): δ=8.08-8.10 (1H, d), 7.97-8.00 (1H, m), 7.26-7.32 (1H, t), 7.09-7.13 (1H, d), 7.03 (1H, s), 6.84-6.88 (1H, d), 4.33-4.40 (1H, d), 4.10-4.18 (1H, m), 3.83-3.92 (1H, d), 3.20-3.30 (1H, t, partially masked by solvent), 2.86-2.97 (1H, t), 2.45 (3H, s), 2.36-2.43 (2H, quart), 1.93-2.06 (2H, m), 1.40-1.60 (2H, m), 1.08-1.13 (3H, t) ppm.

LRMS (electrospray): m/z [M+Na]⁺ 440, [2M+Na]⁺ 857 , [M−H]⁺ 416.

Anal. Found C, 58.03; H, 5.63; N, 9.24. C₂₁H₂₄FN₃O₃S. 0.95 mol H₂O requires C, 57.80; H, 6.03; N, 9.63%.

### Example 12

### 5-Fluoro-2-(3-methylsulfanyl-phenoxy)-N-(1-cyclopropanecarbonyl-piperidin-4-yl)-nicotinamide

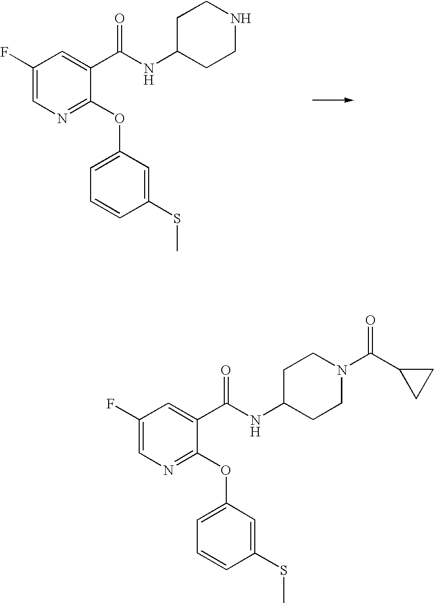

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by cyclopropanecarbonyl chloride (43 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120 μl, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-(1-cyclopropylcarbonyl-piperidin-4-yl)-nicotinamide (100 mg) as an off-white solid.

¹H NMR (400 MHz, CD₃OD): δ=8.07-8.09 (1H, d), 7.97-8.00 (1H, dd), 7.27-7.32 (1H, t), 7.08-7.12 (1H, d), 7.03 (1H, s), 6.85-6.90 (1H, m), 4.20-4.38 (2H, m), 4.12-4.20 (1H, m), 3.22-3.41 (1H, m, partially masked by solvent), 2.88-2.99 (1H, m), 2.45 (3H, s), 2.00-2.09 (1H, m), 1.90-2.00 (2H, m), 1.40-1.63 (2H, m), 0.77-0.86 (4H, m) ppm.

LRMS (electrospray): m/z [M+Na]⁺ 452, [2M+Na]⁺ 881 , [M−H]⁺ 428.

HRMS: Found [M+H]⁺ 430.1597. C₂₂H₂₅FN₃O₃S requires 430.1595 Found [M+Na]⁺ 452.1414. C₂₂H₂₄FN₃O₂SNa requires 452.1414.

### Example 13

### 5-Fluoro-2-(3-methylsulfanyl-phenoxy)-N-(1-isobutyryl-piperidin-4-yl)-nicotinamide

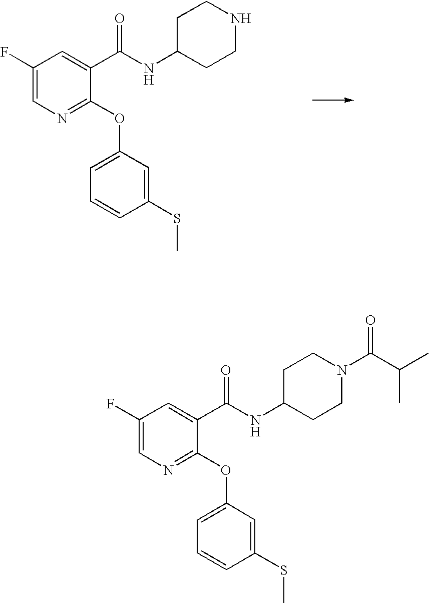

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by isobutyryl chloride (44 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120□l, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-(1-isobutyryll-piperidin-4-yl)-nicotinamide (73 mg) as an off-white solid.

¹H NMR (400 MHz, CD₃OD): δ=8.08-8.10 (1H, d), 7.97-8.00 (1H, m), 7.26-7.32 (1H, t), 7.08-7.13 (1H, d), 7.03 (1H, s), 6.86-6.89 (1H, d), 4.33-4.40 (1H, d), 4.12-4.19 (1H, m), 3.96-4.02 (1H, d), 3.23-3.31 (1H, m, partially masked by solvent), 2.86-2.97 (2H, m), 2.45 (3H, s), 1.93-2.03 (2H, m), 1.40-1.59 (2H, m), 1.04-1.09 (6H, d) ppm.

LRMS (electrospray): m/z [M+Na]⁺ 454, [M−H]⁺ 430.

Anal. Found C, 59.64; H, 5.86; N, 9.28. C₂₂H₂₆FN₃O₃S. 0.66 mol H₂O requires C, 59.58; H, 6.21; N, 9.47%.

### Example 14

### 5-Fluoro-2-(3-methylsulfanyl-phenoxy)-N-(1-butyryl-piperidin-4-yl)-nicotinamide

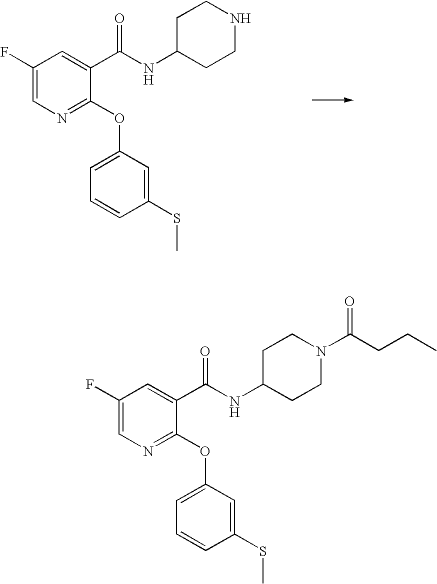

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by butyryl chloride (44 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120□l, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-(1-butyryl-piperidin-4-yl)-nicotinamide (100 mg) as an off-white solid.

¹H NMR (400 MHz, CD₃OD): δ=8.09 (1H, s), 7.97-8.01 (1H, m), 7.28-7.33 (1H, t,), 7.09-7.14 (1H, d), 7.04 (1H, s), 6.86-6.90 (1H, d), 4.31-4.39 (1H, d), 4.10-4.19 (1H, m), 3.87-3.96 (1H, d), 3.20-3.32 (1H, m, partially masked by solvent), 2.85-2.98 (1H, t), 2.45 (3H, s), 2.34-2.39 (2H, t), 1.93-2.05 (2H, m), 1.40-1.63 (2H, sext+m), 0.93-0.98 (3H, t) ppm.

LRMS (electrospray): m/z [M+Na]⁺ 454, [2M+Na]⁺ 885, [M−H]⁺ 431.

Anal. Found C, 60.73; H, 6.09; N, 9.31. C₂₂H₂₆FN₃O₃S. 0.25 mol H₂O requires C, 60.60; H, 6.13; N, 9.64%.

### Example 15

### 5-Fluoro-2-(3-methylsulfanyl-phenoxy)-N-(1-cyclobutylcarbonyl-piperidin-4-yl)-nicotinamide

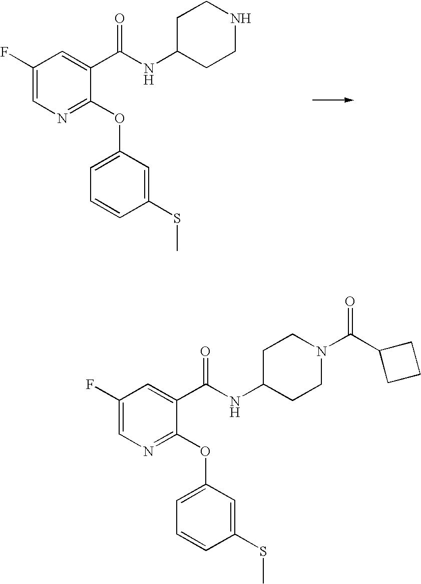

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by cyclobutylcarbonyl chloride (50 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120□l, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-(1-cyclobutylcarbonyl-piperidin-4-yl)-nicotinamide (92 mg) as an off-white solid.

¹H NMR (400 MHz, CD₃OD): δ=8.08-8.10 (1H, d), 7.97-8.01 (1H, m), 7.26-7.32 (1H, t), 7.07-7.13 (1H, d), 7.04 (1H, s), 6.84-6.89 (1H, d), 4.29-4.36 (1H, d), 4.09-4.18 (1H, m), 3.72-3.79 (1H, d), 3.36-3.42 (1H, m), 3.12-3.19 (1H, t), 2.86-2.97 (1H, t), 2.45 (3H, s), 2.12-2.30 (4H, 2xm), 1.92-2.04 (3H, m), 1.78-1.88 (1H, m), 1.40-1.52 (2H, m) ppm.

LRMS (electrospray): m/z [M+Na]⁺ 466, [M−H]⁺ 442.

Anal. Found C, 58.71; H, 5.50; N, 8.92. C₂₃H₂₆FN₃O₃S. 0.5 mol H₂O requires C, 58.94; H, 5.85; N, 8.87%.

### Example 16

### 5-Fluoro-N-[1-(3-methylbutyryl)-piperidin-4-yl)-2-(3-methylsulfanyl-phenoxy)-nicotinamide

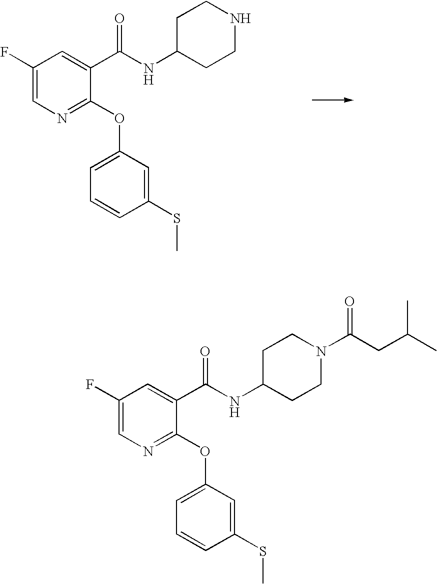

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by 3-methyl-butyryl chloride (50 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120□l, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-N-[1-(3-methylbutyryl)-piperidin-4-yl)-2-(3-methylsulfanyl-phenoxy)-nicotinamide (91 mg) as an off-white solid.

¹H NMR (400 MHz, CD₃OD): δ=8.08-8.10 (1H, d), 7.97-8.00 (1H, m), 7.27-7.32 (1H, t), 7.08-7.12 (1H, d), 7.03 (1H, s), 6.85-6.88 (1H, d), 4.34-4.40 (1H, d), 4.12-4.19 (1H, m), 3.88-3.97 (1H, d), 3.20-3.30 (1H, t, partially masked by solvent), 2.87-2.97 (1H, t), 2.45 (3H, s), 2.23-2.27 (2H, d), 1.93-2.07 (3H, m), 1.40-1.58 (2H, m), 0.94-0.98 (6H, d) ppm.

LRMS (electrospray): m/z [M+Na]⁺ 468, [M−H]⁺ 445.

HRMS: Found [M+H]⁺ 446.1909. C₂₃H₂₉FN₃O₃S requires 446.1908 Found [M+Na]⁺ 468.1727. C₂₃H₂₈FN₃O₃SNa requires 468.1727.

### Example 17

### N-[1-(2,2-Dimethyl-propionyl)-piperidin-4-yl]-5-fluoro-2-(3-methylsulfanyl-phenoxy)-nicotinamide

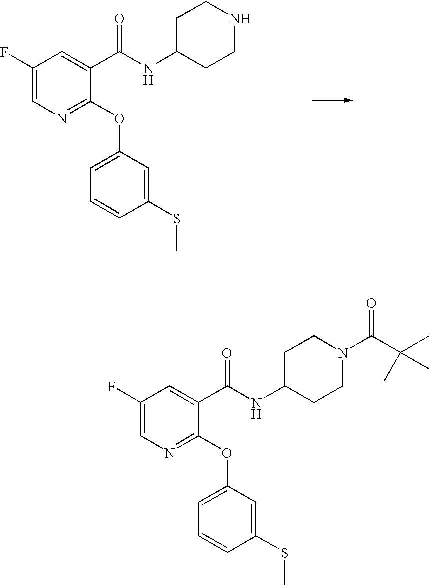

5-Flouro-2-(3methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by tert-butylcarbonyl chloride (50 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120 μl, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give N-[1-(2,2-Dimethyl-propionyl)-piperidin-4-yl]-5-fluoro-2-(3-methylsulfanyl-phenoxy)-nicotinamide (23 mg) as an off-white solid.

¹H NMR (400 MHz, CD₃OD): δ=8.08-8.10 (1H, d), 7.96-8.00 (1H, m), 7.26-7.32 (1H, t), 7.08-7.12 (1H, d), 7.03 (1H, s), 6.85-6.88 (1H, d), 4.23-4.30 (2H, d), 4.10-4.20 (1H, m), 3.05-3.15 (2H, t), 2.45 (3H, s), 1.95-2.04 (2H, m), 1.40-1.55 (2H, m), 1.24 (9H, s) ppm.

LRMS (electrospray): m/z [M+Na]⁺ 468, [M−H]⁺ 444.

Anal. Found C, 60.93; H, 6.30; N, 8.98. C₂₃H₂₈FN₃O₃S. 0.5 mol H₂O requires C, 60.77; H, 6.43; N, 9.24%.

### Example 18

### 5-Fluoro-2-(3-methylsulfanyl-phenoxy)-N-(1-cyclopentylcarbonyl-piperidin-4-yl)-nicotinamide

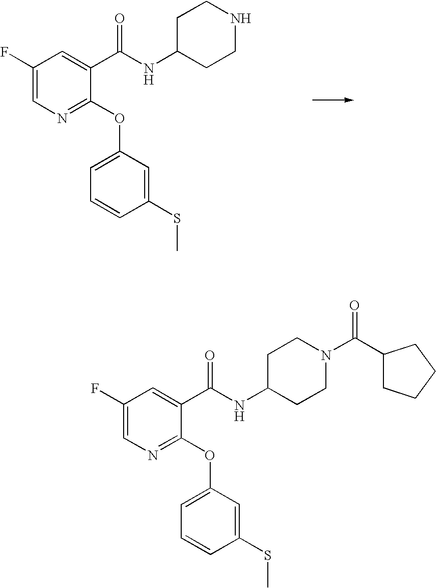

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by cyclopentylcarbonyl chloride (55 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120 μl, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-(1-cyclopentylcarbonyl-piperidin-4-yl)-nicotinamide (105 mg) as an off-white solid.

¹H NMR (400 MHz, CD₃OD): δ=8.09 (1H, s), 7.95-7.99 (1H, m), 7.27-7.33 (1H, t), 7.09-7.14 (1H, d), 7.04 (1H, s), 6.84-6.91 (1H, d), 4.30-4.40 (1H, m), 4.09-4.19 (1H, m), 3.94-4.06 (1H, m), 3.20-3.35 (1H, m, partially masked by solvent), 3.00-3.10 (1H, m), 2.86-2.98 (1H, m), 2.46 (3H, s), 1.90-2.10 (2H, m), 1.47-1.90 (8H, 3xm), 1.02 (2H, s) ppm.

LRMS (electrospray): m/z [M+Na]⁺ 480, [M−H]⁺ 456.

Anal. Found C, 62.33; H, 6.23; N, 8.82. C₂₄H₂₈FN₃O₃S. 0.25 mol H₂O requires C, 62.39; H, 6.22; N, 9.09%.

### Example 19

### Acetic acid 2-(4-{[5-fluoro-2-(3-methylsulfanyl-phenoxy)-pyridine-3-carbonyl]-amino}-piperidin-1-yl)-2-oxo-ethyl ester

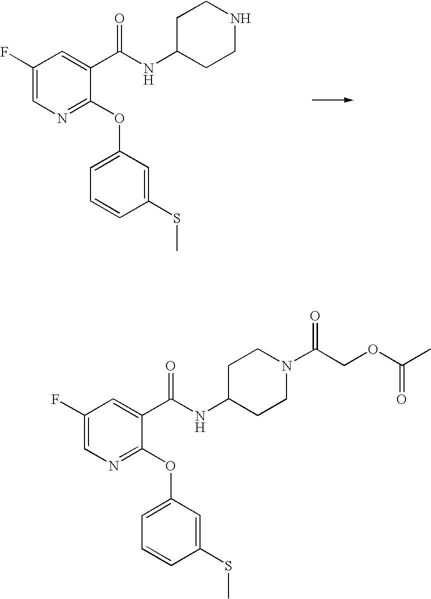

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by acetic acid chlorocarbonyl methyl ester (56 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120 ml, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane methanol:concentrated aqueous ammonia (99.5:0.5:0.05 changing to 95:5:0.5, by volume) to give Acetic acid 2-(4-({[5-fluoro-2-(3-methylsulfanyl-phenoxy)-pyridine-3-carbonyl]-amino}-piperidin-1-yl)-2-oxo-ethyl ester (81 mg) as an off-white solid.

¹H NMR (400 MHz, CD₃OD): δ=8.08-8.10 (1H, d), 7.97-8.00 (1H, dd), 7.28-7.32 (1H, t), 7.06-7.13 (1H, d), 7.03 (1H, s), 6.85-6.90 (1H, d), 4.75-4.84 (2H, quart, partially masked by solvent), 4.26-4.34 (1H, d), 4.12-4.18 (1H, m), 3.72-3.81 (1H, d), 3.18-3.29 (1H, m, partially masked by solvent), 2.90-3.00 (1H, d), 2.45 (3H, s), 2.10 (3H, s), 1.95-2.09 (2H, m), 1.46-1.63 (2H, m) ppm.

LRMS (electrospray): m/z [M+Na]⁺ 484, [2M+Na]⁺ 945, [M−H]⁺ 460.

Anal. Found C, 56.76; H, 65.26; N, 8.64. C₂₂H₂₄FN₃O₅S. 0.2 mol H₂O requires C, 56.81; H, 5.29; N, 9.03%.

### Example 20

### 5-Fluoro-2-(3-methylsulfanyl-phenoxy)-N-[1-(3-methylsulfanyl-propionyl)-piperidin-4-yl]-nicotinamide

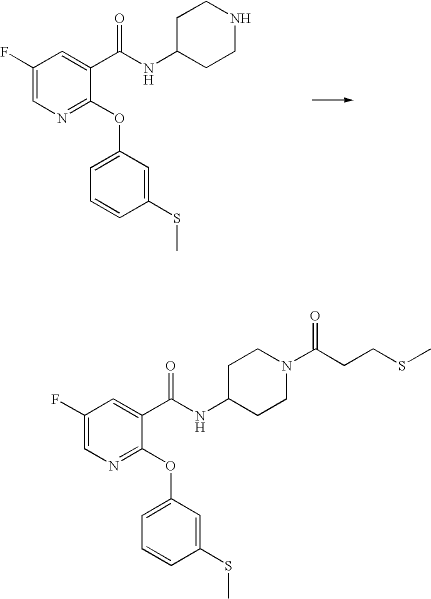

5-Fluoro-2-(3-methylsulfanyl-phenoxy)-N-piperidin-4-yl-nicotinamide (100 mg, 0.251 mmol) and N,N-dimethylaminopyridine (5 mg) were dissolved in dichloromethane (3 ml) under nitrogen at room temperature and triethylamine (120□l, 0.753 mmol) was added followed by 1-(3-methylsulfanyl)-propionyl chloride (57 mg, 0.42 mmol). The reaction was allowed to stir at room temperature for 2 h and 1-(3-Dimethylaminopropyl)-3-ethylcarbodiimide hydrochloride (80 mg, 0.377 mmol) followed by triethylamine (120 μl, 0.753 mmol). The reaction was stirred at room temperature for 18 h, quenched with sat. ammonium chloride solution (0.5 ml), diluted with water (3 ml) and the organic layer was removed via a separation tube. The solvent was removed under reduced pressure and the residue was by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(3-methylsulfanyl-propionyl)-piperidin-4-yl]-nicotinamide (103 mg) as an off-white solid.

¹H NMR (400 MHz, CD₃OD): δ=8.09 (1H, s), 7.97-8.00 (1H, m), 7.27-7.32 (1H, t), 7.09-7.14 (1H, d), 7.03 (1H, s), 6.84-6.88 (1H, d), 4.32-4.38 (1H, d), 4.11-4.19 (1H, m), 3.89-3.95 (1H, d), 3.22-3.30 (1H, m, partially masked by solvent), 2.88-2.98 (1H, t), 2.63-2.77 (4H, m), 2.45 (3H, s), 2.09 (3H, s), 1.94-2.08 (2H, m), 1.42-1.60 (2H, m) ppm.

LRMS (electrospray): m/z [M+Na]⁺ 486, [2M+Na]⁺ 949, [M−H]⁺ 462.

Anal. Found C, 55.66; H, 5.58; N, 8.56. C₂₂H₂₆FN₃O₃S. 0.66 mol H₂O requires C, 55.51; H, 5.46; N, 8.83%.

### Example 21

### N-[1-(2-Dimethylamino-acetyl)-piperidin-4-yl]-5-fluoro-2-(3-methylsulfanyl-phenoxy)-nicotinamide

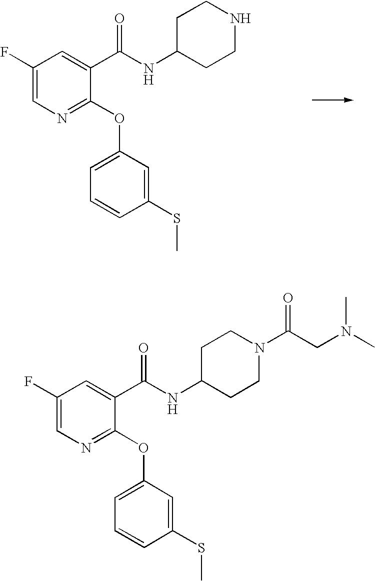

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67 ml, 0.611 mmol) were added to a solution of 2-dimethylaminoacetic acid (43 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give N-[1-(2-Dimethylamino-acetyl)-piperidin-4-yl]-5-fluoro-2-(3-methylsulfanyl-phenoxy)-nicotinamide (90 mg) as an off-white solid.

¹H NMR (400 MHz, CD₃OD): δ=8.08-8.10 (1H, d), 7.97-8.00 (1H, m), 7.27-7.32 (1H, t), 7.09-7.13 (1H, d), 7.03 (1H, s), 6.84-6.88 (1H, d), 4.26-4.36 (1H, d), 4.09-4.19 (1H, m), 3.95-4.01 (1H, d), 3.18-3.32 (3H, m, partially masked by solvent), 2.90-2.98 (1H, t), 2.46 (3H, s), 2.30 (6H, s), 1.94-2.04 (2H, m), 1.40-1.61 (2H, m) ppm.

LRMS (electrospray): m/z [M+H]⁺ 447, [M−H]⁺ 445.

Anal. Found C, 55.14; H, 5.92; N, 11.07. C₂₂H₂₇FN₄O₃S. 0.33 mol CH₂Cl₂. 0.75 mol H₂O requires C, 54.98; H, 6.02; N, 11.47%.

### Example 22

### N-[1-(2-Ethoxy-acetyl)-piperidin-4-yl]-5-fluoro-2-(3-methylsulfanyl-phenoxy)-nicotinamide

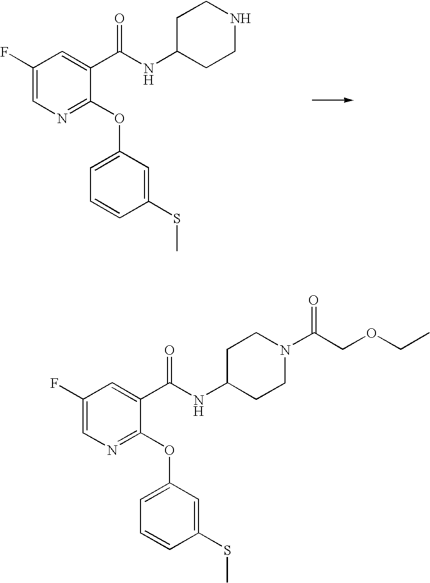

5-Fluoro-2-(3-methylsulfanyl-phenoxy)-N-piperidin-4-yl-nicotinamide (110 mg , 0.276 mmol), 1-hydroxybenzotriazole (46 mg, 0.304 mmol) and N-methylmorpholine (67□l, 0.611 mmol) were added to a solution of 2-ethoxyacetic acid (43 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol concentrated aqueous ammonia (99.5:0.5:0.05 changing to 95:5:0.5, by volume) to give N-[1-(2-ethoxy-acetyl)-piperidin-4-yl]-5-fluoro-2-(3-methylsulfanyl-phenoxy)-nicotinamide (96 mg) as an off-white solid.

¹H NMR (400 MHz, CD₃OD): δ=8.08-8.10 (1H, d), 7.97-8.00 (1H, m), 7.28-7.33 (1H, t), 7.08-7.12 (1H, d), 7.04 (1H, s), 6.84-6.88 (1H, d), 4.27-4.36 (1H, d), 4.10-4.21 (3H, quart+m), 3.81-3.91 (1H, d), 3.49-3.58 (2H, quart), 3.17-3.29 (1H, t), 2.90-2.99 (1H, t), 2.45 (3H, s), 1.96-2.07 (2H, t), 1.42-1.62 (2H, m), 1.17-1.22 (3H, t) ppm.

LRMS (electrospray): m/z [M+Na]⁺ 470, [2M+H]⁺ 917, [M−H]⁺ 446.

Anal. Found C, 58.22; H, 5.80; N, 9.08. C₂₂H₂₆FN₃O₄S. 0.33 mol H₂O requires C, 58.26; H, 5.93; N, 9.27%.

### Example 23

### 5-Fluoro-2-(3-methylsulfanyl-phenoxy)-N-[1-(tetrahydro-furan-3-carbonyl)-piperidin-4-yl]-nicotinamide

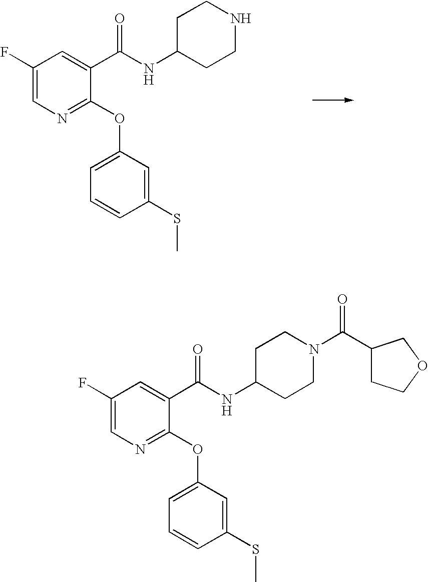

5-Fluoro-2-(3-methylsulfanyl-phenoxy)-N-piperidin-4-yl-nicotinamide (110 mg , 0.276 mmol), 1-hydroxybenzotriazole (46 mg, 0.304 mmol) and N-methylmorpholine (67□l, 0.611 mmol) were added to a solution of tetrahydrofuran-3-carboxylic acid (48 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-Fluoro-2-(3-methylsulfanyl-phenoxy)-N-[1-(tetrahydro-furan-3-carbonyl)-piperidin-4-yl]-nicotinamide (85 mg) as an off-white solid.

¹H NMR (400 MHz, CD₃OD): δ=8.08-8.10 (1H, d), 7.97-8.01 (1H, m), 7.27-7.32 (1H, t), 7.08-7.11 (1H, d), 7.03 (1H, s), 6.84-6.88 (1H, d), 4.12-4.20 (1H, d), 4.10-4.20 (1H, m), 3.90-4.02 (2H, t+d), 3.74-3.85 (3H, m), 3.38-3.46 (1H, m), 3.24-3.36 (1H, m, partially masked by solvent), 2.88-2.98 (1H, m), 2.45 (3H, s), 1.95-2.16 (4H, m), 1.40-1.60 (2H, m) ppm.

LRMS (electrospray): m/z [M+Na]⁺ 482, [M−H]⁺ 458.

Anal. Found C, 59.73; H, 5.73; N, 8.93. C₂₃H₂₆FN₃O₄S requires C, 60.12; H, 5.70; N, 9.14%.

### Example 24

### N-[1-(2-Acetyl-amino-acetyl)-piperidin-4-yl]-5-fluoro-2-(3-methylsulfanyl-phenoxy)-nicotinamide

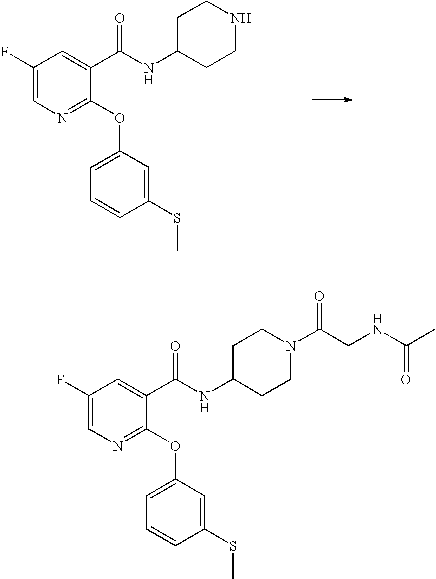

5-Fluoro-2-(3-methylsulfanyl-phenoxy)-N-piperidin-4-yl-nicotinamide (110 mg , 0.276 mmol), 1-hydroxybenzotriazole (46 mg, 0.304 mmol) and N-methylmorpholine (67□l, 0.611 mmol) were added to a solution of 2-acetyl-amino-acetic acid (49 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give N-[1-(2-acetyl-amino-acetyl )-piperidin-4-yl]-5-fluoro-2-(3-methylsulfanyl-phenoxy)-nicotinamide (96 mg) as an off-white solid.

¹H NMR (400 MHz, CD₃OD): δ=8.09-8.11 (1H, d), 7.97-8.00 (1H, m), 7.27-7.34 (1H, t), 7.10-7.14 (1H, d), 7.04 (1H, s), 6.86-6.90 (1H, d), 4.30-4.38 (1H, d), 4.06-4.19 (2H, d+m), 3.97-4.02 (1H, d), 3.79-3.87 (1H, d), 3.20-3.30 (1H, m, partially masked by solvent), 2.90-3.00 (1H, t), 2.45 (3H, s), 1.96-2.07 (5H, s+m), 1.42-1.63 (2H, m) ppm.

LRMS (electrospray): m/z [M+Na]⁺ 483, [M−H]⁺ 459.

Anal. Found C, 56.61; H, 5.57; N, 11.70. C₂₂H₂₅FN₄O₄S. 0.33 mol H₂O requires C, 56.64; H, 5.55; N, 12.01%.

### Example 25

### N-(1-Benzoyl-piperidin-4-yl)-5-fluoro-2-(3-methylsulfanyl-phenoxy)-nicotinamide

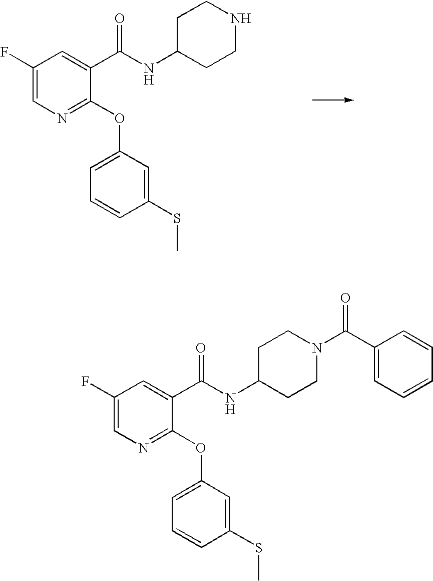

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67□l, 0.611 mmol) were added to a solution of benzoic acid (51 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give N-(1-Benzoyl-piperidin-4-yl )-5-fluoro-2-(3-methylsulfanyl-phenoxy)-nicotinamide (105 mg) as an off-white solid.

¹H NMR (400 MHz, CD₃OD): δ=8.13 (1H, s), 7.95-8.02 (1H, dd), 7.41-7.49 (3H, m), 7.35-7.40 (2H, m), 7.27-7.32 (1H, t), 7.08-7.13 (1H, d), 7.05 (1H, s), 6.85-6.90 (1H, d), 4.40-4.55 (1H, brs), 4.10-4.21 (1H, m), 3.62-3.77 (1H, brs), 3.07-3.25 (2H, brs), 2.45 (3H, s), 1.87-2.14 (2H, m), 1.48-1.70 (2H, m) ppm.

LRMS (electrospray): m/z [M+Na]⁺ 488, [M−H]⁺ 464.

Anal. Found C, 63.83; H, 5.21; N, 8.89. C₂₅H₂₄FN₃O₃S. 0.25 mol H₂O requires C, 63.88; H, 5.25; N, 8.94%.

### Example 26

### 5-Fluoro-2-(3-methylsulfanyl-phenoxy)-N-[1-(pyridine-4-carbonyl)-piperidin-4-yl]-nicotinamide

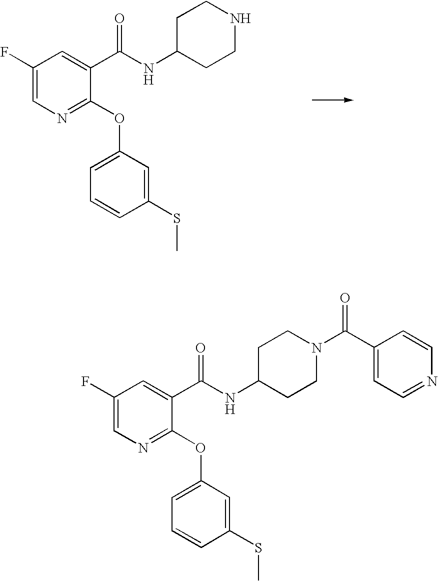

5-Fluoro-2-(3-methylsulfanyl-phenoxy)-N-piperidin-4-yl-nicotinamide (110 mg , 0.276 mmol), 1-hydroxybenzotriazole (46 mg, 0.304 mmol) and N-methylmorpholine (67□l, 0.611 mmol) were added to a solution of pyridine-4-carboxylic acid (51 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(pyridine-4-carbonyl)-piperidin-4-yl]-nicotinamide (95 mg) as an off-white solid.

¹H NMR (400 MHz, CD₃OD): δ=8.60-8.63 (2H, d), 8.10-8.12 (1H, d), 7.98-8.02 (1H, dd), 7.40-7.43 (2H, d), 7.30-7.34 (1H, t), 7.11-7.15 (1H, d), 7.05 (1H, s), 6.85-6.89 (1H, d), 4.44-4.52 (1H, m), 4.16-4.22 (1H, m), 3.53-3.62 (1H, m), 3.21-3.20 (1H, m, partially masked by solvent), 3.11-3.20 (1H, m), 2.45 (3H, s), 2.05-2.12 (1H, m), 1.92-2.00 (1H, m), 1.50-1.69 (2H, m) ppm.

LRMS (electrospray): m/z [M+Na]⁺ 489, [M−H]⁺ 465.

Anal. Found C, 61.00; H, 5.07; N, 11.56. C₂₄H₂₃FN₄O₃S. 0.33 mol H₂O requires C, 61.00; H, 5.05; N, 11.86%.

### Example 27

### 5-Fluoro-2-(3-methylsulfanyl-phenoxy)-N-[1-(pyridine-3-carbonyl)-piperidin-4-yl]-nicotinamide

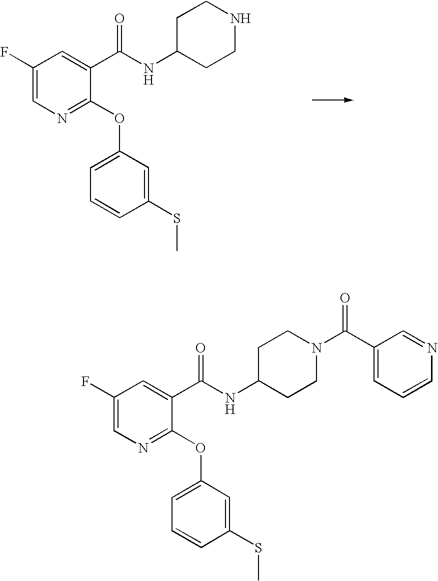

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67□l, 0.611 mmol) were added to a solution of pyridine-3-carboxylic acid (51 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(pyridine-3-carbonyl)-piperidin-4-yl]-nicotinamide (97 mg) as an off-white solid.

¹H NMR (400 MHz, CD₃OD): δ=8.60-8.64 (1H, d), 8.59 (1H, s), 8.10-8.13 (1H, d), 7.98-8.02 (1H, dd), 7.85-7.89 (1H, m), 7.48-7.53 (1H, m), 7.29-7.35 (1H, t), 7.11-7.14 (1H, d), 7.03 (1H, s), 6.85-6.89 (1H, d), 4.414.55 (1H, brs), 4.15-4.22 (1H, m), 3.57-3.70 (1H, brs), 3.10-3.40 (2H, 2xbrs, partially masked by solvent), 2.45 (3H, s), 1.94-2.17 (1H, m), 1.53-1.69 (2H, m) ppm.

LRMS (electrospray): m/z [M+Na]⁺ 489, [M−H]⁺ 465.

Anal. Found C, 61.08; H, 4.84; N, 11.53. C₂₄H₂₃FN₄O₃S. 0.25 mol H₂O requires C, 61.20; H, 5.03; N, 11.89%.

### Example 28

### 5-Fluoro-2-(3-methylsulfanyl-phenoxy)-N-[1-(pyridine-2-carbonyl)-piperidin-4-yl]-nicotinamide

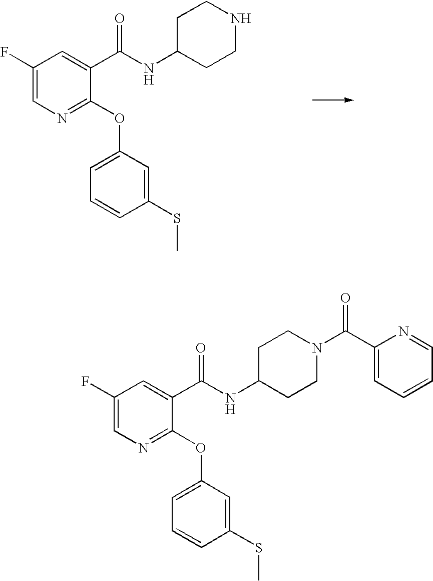

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67□l, 0.611 mmol) were added to a solution of pyridine-2-carboxylic acid (51 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(pyridine-2-carbonyl)-piperidin-4-yl]-nicotinamide (100 mg) as an off-white solid.

¹H NMR (400 MHz, CD₃OD): δ=8.56-8.59 (1H, d), 8.09-8.11 (1H, d), 7.96-8.00 (1H, dd), 7.90-7.95 (1H, t), 7.56-7.59 (1H, d), 7.47-7.50 (1H, m), 7.27-7.33 (1H, t), 7.08-7.12 (1H, d), 7.04 (1H, s), 6.85-6.89 (1H, m), 4.45-4.54 (1H, d), 4.16-4.23 (1H, m), 3.63-3.71 (1H, d), 3.12-3.28 (2H, m), 2.44 (3H, s), 2.05-2.14 (1H, d), 1.90-1.99 (1H, d), 1.57-1.71 (2H, m) ppm.

LRMS (electrospray): m/z [M+Na]⁺ 489, [M−H]⁺ 465.

Anal. Found C, 61.33; H, 4.97; N, 11.67. C₂₄H₂₃FN₄O₃S. 0.15 mol H₂O requires C, 61.43; H, 5.01; N, 11.94%.

### Example 29

### 5-Fluoro-2-(3-methylsulfanyl-phenoxy)-N-[1-(pyrimidine-5-carbonyl)-piperidin-4-yl]-nicotinamide

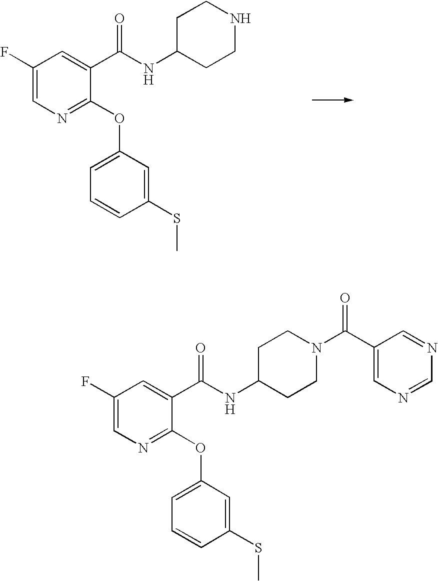

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67□l, 0.611 mmol) were added to a solution of pyrimidine-5-carboxylic acid (52 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane: methanol:concentrated aqueous ammonia (99.5:0.5:0.05 changing to 95:5:0.5, by volume) to give 5-fluoro-2-(3-methylsulfanyl-phenoxy)-N-[1-(pyrimidine-5-carbonyl)-piperidin-4-yl]-nicotinamide (40 mg) as an off-white solid.

¹H NMR (400 MHz, CD₃OD): δ=9.21 (1H, s), 8.82 (2H, s), 8.11-8.13 (1H, d), 7.98-8.02 (1H, m), 7.30-7.35 (1H, t), 7.11-7.15 (1H, d), 7.04 (1H, s), 6.84-6.88 (1H, d), 4.41-4.58 (1H, brs), 4.16-4.22 (1H, m), 3.61-3.75 (1H, brs), 3.12-3.40 (2H, m, partially masked by solvent), 2.45 (3H, s), 1.96-2.16 (2H, m), 1.56-1.72 (2H, m) ppm.

LRMS (electrospray): m/z [M−H]⁺ 466.

### Example 30

### 5-Fluoro-2-(3-methylsulfanyl-phenoxy)-N-[1-(2-methyl-pyridine-3-carbonyl)-piperidin-4-yl]-nicotinamide

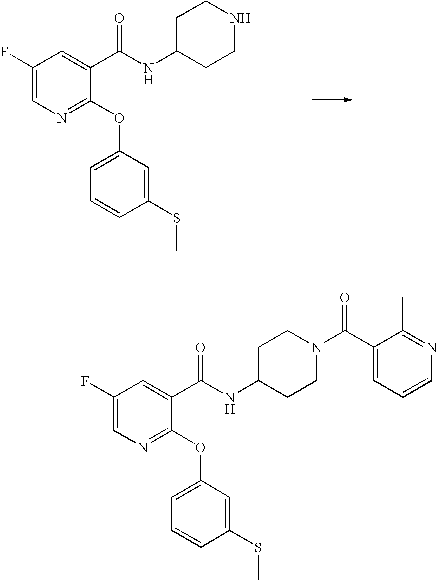

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67□l, 0.611 mmol) were added to a solution of 2-methyl-pyridine-3-carboxylic acid (57 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(2-methyl-pyridine-3-carbonyl)-piperidin-4-yl]-nicotinamide (110 mg ) as an off-white solid.

¹H NMR (400 MHz, CD₃OD): δ=8.45-8.48 (1H, d), 8.10-8.12 (1H, d), 7.96-8.02 (1H, d), 7.63-7.67 (1H, d), 7.28-7.34 (2H, m), 7.08-7.13 (1H, d), 7.03 (1H, s), 6.85-6.89 (1H, d), 4.40-4.60 (1H, brs), 4.12-4.22 (1H, m), 3.38-3.46 (1H, m), 3.10-3.30 (2H, m, partially masked by solvent), 2.44-2.50 (6H, 2xs), 2.04-2.14 (1H, d), 1.88-1.96 (1H, d), 1.59-1.71 (1H, m), 1.46-1.58 (1H, m) ppm.

LRMS (electrospray): m/z [M+Na]⁺ 503, [M−H]⁺ 479.

Anal. Found C, 61.52; H, 5.34; N, 11.45. C₂₅H₂₅FN₄O₃S. 0.4 mol H₂O requires C, 61.50; H, 5.12; N, 11.48%.

### Example 31

### 5-Fluoro-2-(3-methylsulfanyl-phenoxy)-N-[1-(2-methyl-benzoyl)-piperidin-4-yl]-nicotinamide

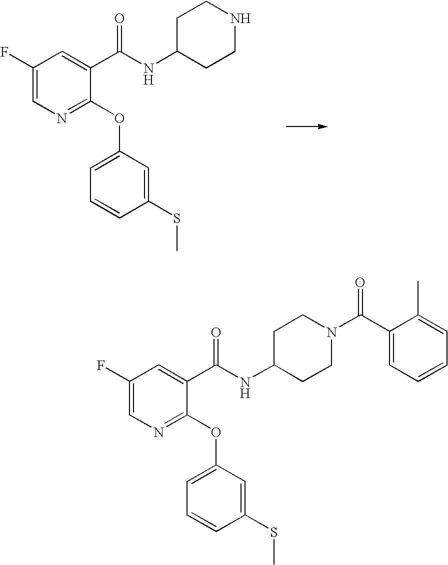

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67 μl, 0.611 mmol) were added to a solution of 2-methyl-benzoic acid (57 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(2-methyl-benzoyl)-piperidin-4-yl]-nicotinamide (110 mg) as an off-white solid.

¹H NMR (400 MHz, CD₃OD): δ=8.08-8.10 (1H, d), 7.96-8.00 (1H, brs), 7.20-7.31 (4H, m), 7.07-7.19 (2H, m+d), 7.05 (1H, s), 6.85-6.90 (1H, d), 4.40-4.60 (1H, 2xm), 4.12-4.20 (1H, m), 3.39-3.46 (1H, m), 3.07-3.24 (2H, m), 2.44 (3H, s), 2.03-2.13 (1H, d), 1.83-1.93 (1H, m), 1.57-1.69 (1H, m), 1.42-1.57 (1H, m) ppm.

LRMS (electrospray): m/z [M+Na]⁺ 502, [M−H]⁺ 478.

Anal. Found C, 62.94; H, 5.41; N, 8.34. C₂₆H₂₆FN₃O₃S. 1 mol H₂O requires C, 62.76; H, 5.67; N, 8.44%.

### Example 32

### N-(1-Cyclohexylcarbonyl-piperidin-4-yl)-5-fluoro-2-(3-methylsulfanyl-phenoxy)-nicotinamide

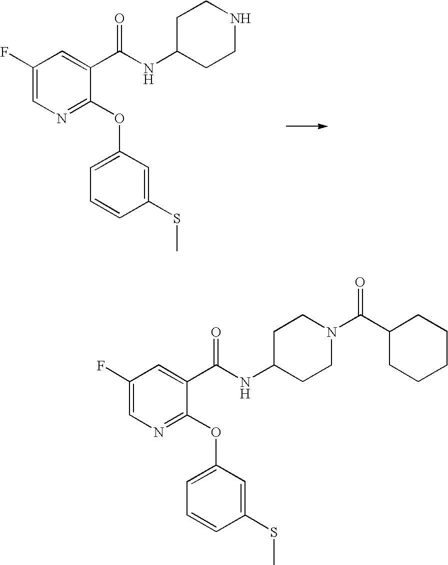

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67 μl, 0.611 mmol) were added to a solution of cyclohexylcarboxylic acid (53 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give N-(1-Cyclohexylcarbonyl-piperidin-4-yl)-5-fluoro-2-(3-methylsulfanyl-phenoxy)-nicotinamide (37 mg) as an off-white solid.

¹H NMR (400 MHz, CD₃OD): δ=8.09 (1H, s), 7.97-8.00 (1H, m), 7.27-7.32 (1H, t), 7.08-7.13 (1H, d), 7.03 (1H, s), 6.84-6.88 (1H, d), 4.31-4.39 (1H, d), 4.10-4.19 (1H, m), 3.93-3.99 (1H, d), 3.20-3.30 (1H, m, partially masked by solvent), 2.84-2.96 (1H, t), 2.59-2.66 (1H, t), 2.44 (3H, s), 2.00-2.07 (1H, d), 1.92-1.99 (1H, d), 1.64-1.80 (5H, 2xd), 1.18-1.59 (7H, m) ppm.

LRMS (electrospray): m/z [M+Na]⁺ 494, [M−H]⁺ 470.

Anal. Found C, 63.04; H, 6.46; N, 8.97. C₂₅H₃₀FN₃O₃S requires C, 63.07; H, 6.46; N, 8.83%.

### Example 33

### 5-Fluoro-2-(3-methylsulfanyl-phenoxy)-N-[1-(5-oxo-pyrrolidine-2-carbonyl)-piperidin-4-yl]-nicotinamide

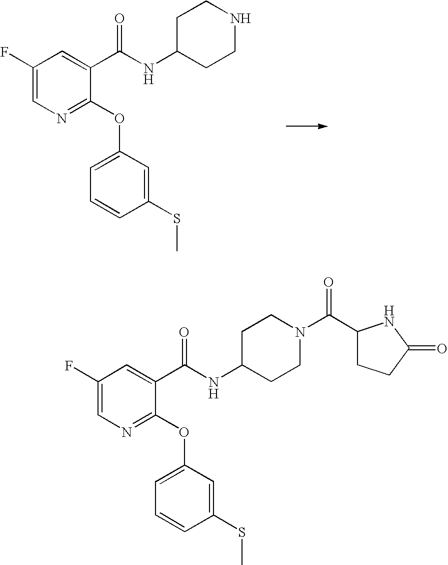

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67 μl, 0.611 mmol) were added to a solution of 5-oxo-pyrrolidine-2-carboxylic acid (54 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-2-(3-methylsulfanyl-phenoxy)-N-[1-(5-oxo-pyrrolidine-2-carbonyl)-piperidin-4-yl]-nicotinamide (100 mg) as an off-white solid.

¹H NMR (400 MHz, CD₃OD): δ=8.08-8.10 (1H, d), 7.98-8.02 (1H, m), 7.29-7.34 (1H, t), 7.10-7.14 (1H, d), 7.04 (1H, s), 6.84-6.88 (1H, d), 4.65-4.71 (1H, m), 4.30-4.38 (1H, m), 4.12-4.20 (1H, m), 3.83-3.92 (1H, m), 3.27-3.36 (2H, m, partially masked by solvent), 2.92-3.02 (1H, m), 2.42-2.54 (4H, s+m), 2.28-2.38 (2H, m), 1.95-2.10 (3H, m), 1.45-1.62 (2H, m) ppm.

LRMS (electrospray): m/z [M+Na]⁺ 495, [M−H]⁺ 472.

Anal. Found C, 57.54; H, 5.50; N, 11.41. C₂₅H₂₅FN₄O₄S. 0.4 mol H₂O requires C, 57.58; H, 5.42; N, 11.68%.

### Example 34

### 5-Fluoro-N-[1-(2-methanesulfonyl-acetyl)-piperidin-4-yl]-2-(3-methylsulfanyl-phenoxy)-nicotinamide

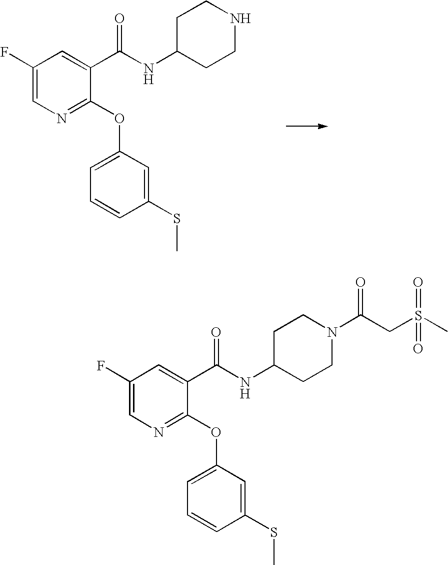

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67 μl, 0.611 mmol) were added to a solution of 2-methanesulfonyl-acetic acid (57 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-N-[1-(2-methanesulfonyl-acetyl)-piperidin-4-yl]-2-(3-methylsulfanyl-phenoxy)-nicotinamide (85 mg) as an off-white solid.

¹H NMR (400 MHz, CD₃OD): δ=8.10-8.11 (1H, d), 7.97-8.00 (1H, m), 7.28-7.33 (1H, t), 7.08-7.13 (1H, d), 7.04 (1H, s), 6.95-6.99 (1H, d), 4.36-4.41 (1H, d), 4.12-4.20 (1H, m), 3.97-4.03 (1H, d), 3.10-3.20 (1H, m, partially masked by solvent), 3.09 (3H, s), 2.98-3.04 (1H, m), 2.44 (3H, s), 1.98-2.08 (2H, t), 1.60-1.72 (1H, m), 1.44-1.58 (1H, m) ppm.

LRMS (electrospray): m/z [M+Na]⁺ 505, [M−H]⁺ 481.

Anal. Found C, 52.15; H, 5.08; N, 8.56. C₂₁H₂₄FN₃O₅S₂ requires C, 52.38; H, 5.02; N, 8.73%.

### Example 35

### 5-Fluoro-N-[1-(2-hydroxy-acetyl)-piperidin-4-yl]-2-(3-methylsulfanyl-phenoxy)-nicotinamide

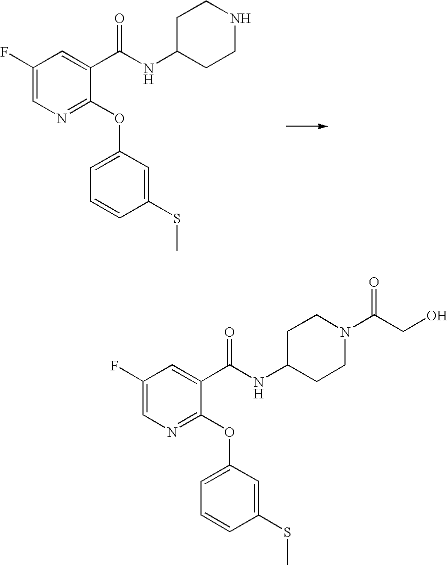

5-Fluoro-2-(3-methylsulfanyl-phenoxy)-N-piperidin-4-yl-nicotinamide (110 mg, 0.276 mmol), 1-hydroxybenzotriazole (46 mg, 0.304 mmol) and N-methylmorpholine (67 μl, 0.611 mmol) were added to a solution of 2-hydroxy-acetic acid (30 mg, 0.42 mmol) in dichloromethane (2.5 ml) under nitrogen at room temperature and 1-(3-dimethylaminopropyl)-3-ethylcarbodiimide hydrochloride (89 mg, 0.468 mmol) was added. The reaction was stirred at room temperature for 18 h and quenched with sat. ammonium chloride (0.5 ml) and diluted with water (3 ml). The organic phase was collected via a hydrophobic separation cartridge, concentrated under reduced pressure and the residue was purified by flash column chromatography on a biotage system eluting with a solvent gradient of dichloromethane:methanol:concentrated aqueous ammonia (99.5:0.5:0.05 changing to 95:5:0.5, by volume) to give 5-fluoro-N-[1-(2-hydroxy-acetyl)-piperidin-4-yl]-2-(3-methylsulfanyl-phenoxy)-nicotinamide (113 mg) as an off-white solid.

¹H NMR (400 MHz, CD₃OD): δ=8.08-8.09 (1H, d), 7.98-8.00 (1H, dd), 7.28-7.34 (1H, t), 7.09-7.13 (1H, d), 7.03-7.04 (1H, m), 6.85-6.89 (1H, m), 4.30-4.39 (1H, d), 4.20 (2H, s), 4.10-4.19 (1H, m), 3.66-3.75 (1H, d), 3.17-3.22 (1H, t), 2.92-3.01 (1H, t), 2.45 (3H, s), 1.96-2.04 (2H, m), 1.42-1.60 (2H, m) ppm.

LRMS (electrospray): m/z [M+Na]⁺ 442, [M−H]⁺ 418.

Anal. Found C, 54.87; H, 5.11; N, 9.18. C₂₀H₂₂FN₃O₄S. 0.33 mol CH₂Cl₂ requires C, 54.54; H, 5.20; N, 9.38%.

### Example 36

### N-[1-(4-Chloro-2-hydroxy-benzoyl)-piperidin-4-yl]-5-fluoro-2-(3-methylsulfanyl-phenoxy)-nicotinamide

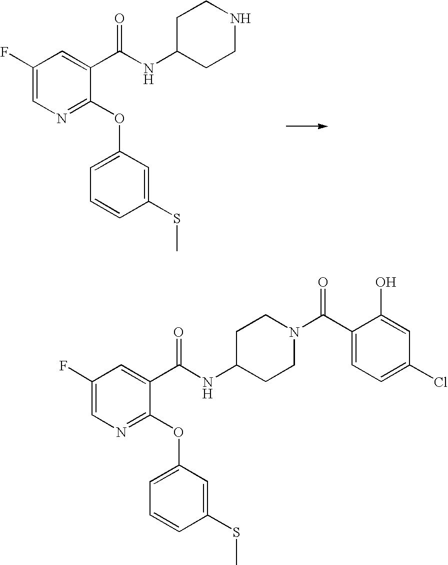

4-Chloro-2-hydroxy-benzoic acid (77 mg, 0.45 mmol) was added to a solution of 5-fluoro-2-(3-methylsulfanyl-phenoxy)-N-piperidin-4-yl-nicotinamide (180 mg, 0.499 mmol), 1-hydroxybenzotriazole (71 mg, 0.523 mmol) and N-methylmorpholine (219 μl, 2.0 mmol) in dichloromethane (2 ml) under nitrogen at room temperature. 1-(3-Dimethylaminopropyl)-3-ethylcarbodiimide hydrochloride (143 mg, 0.746 mmol) was then added and the reaction was stirred at room temperature for 20 h. After quenching with sat. ammonium chloride (1 ml) and diluting with water (3 ml), the organic phase was collected via a hydrophobic separation cartridge, concentrated under reduced pressure and the residue was purified by flash column chromatography on a biotage system eluting with a solvent gradient of ethyl acetate:cyclohexane (15:85 changing to 80:20, by volume) to give N-[1-(4-Chloro-2-hydroxy-benzoyl)-piperidin-4-yl]-5-fluoro-2-(3-methylsulfanyl-phenoxy)-nicotinamide (65 mg) as an off-white foam.

¹H NMR (400 MHz, CD₃OD): δ=8.08-8.10 (1H, d), 7.98-8.01 (1H, dd), 7.29-7.36 (1H, t), 7.11-7.17 (2H, s+d), 7.03-7.05 (1H, m), 6.84-6.91 (3H, m), 4.14-4.22 (1H, m), 3.13-3.21 (2H, t), 2.45 (3H, s), 1.96-2.05 (2H, m), 1.57-1.66 (2H, m) ppm.

LRMS (electrospray): m/z [M+Na]⁺ 538, [M−H]⁺ 514.

HRMS: Found [M+H]⁺ 516.1136. C₂₅H₂₄ClFN₃O₄S requires 516.1155 Found [M+Na]⁺ 538.0952. C₂₅H₂₃ClFN₃O₄SNa requires 538.0974.

### Example 37

### 5-Fluoro-N-[1-(2-hydroxy-4-methyl-benzoyl)-piperidin-4-yl]-2-(3-methylsulfanyl-phenoxy)-nicotinamide

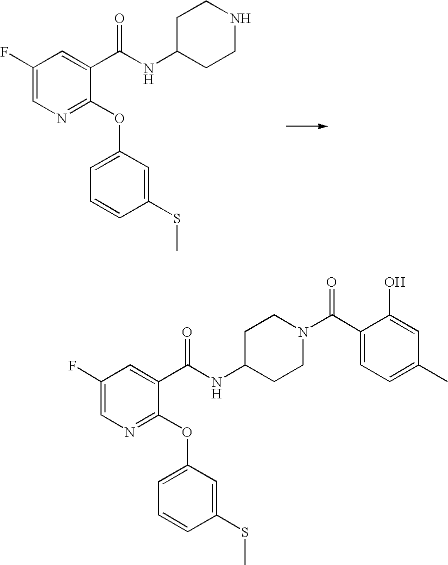

2-Hydroxy-4-methyl-benzoic acid (68 mg, 0.45 mmol) was added to a solution of 5-fluoro-2-(3-methylsulfanyl-phenoxy)-N-piperidin-4-yl-nicotinamide (180 mg, 0.499 mmol), 1-hydroxybenzotriazole (71 mg, 0.523 mmol) and N-methylmorpholine (219 μl, 2.0 mmol) in dichloromethane (2 ml) under nitrogen at room temperature. 1-(3-Dimethylaminopropyl)-3-ethylcarbodiimide hydrochloride (143 mg, 0.746 mmol) was then added and the reaction was stirred at room temperature for 20 h. After quenching with sat. ammonium chloride (1 ml) and diluting with water (3 ml), the organic phase was collected via a hydrophobic separation cartridge, concentrated under reduced pressure and the residue was purified by flash column chromatography on a biotage system eluting with a solvent gradient of ethyl acetate:cyclohexane (15:85 changing to 80:20, by volume) to give 5-fluoro-N-[1-(2-hydroxy-4-methyl-benzoyl)-piperidin-4-yl]-2-(3-methylsulfanyl-phenoxy)-nicotinamide (50 mg) as an off-white foam.

¹H NMR (400 MHz, CD₃OD): δ=8.10-8.12 (1H, d), 7.98-8.02 (1H, dd), 7.26-7.35 (1H, m), 7.10-7.14 (1H, d), 7.04-7.07 (1H, m), 7.03 (1H, s), 6.86-6.90 (1H, m), 6.68-6.92 (1H, d), 6.67 (1H, s), 4.03-4.20 (1H, m), 3.13-3.20 (2H, t), 2.45 (3H, s), 2.28 (3H, s), 1.94-2.03 (2H, m), 1.56-1.66 (2H, m) ppm.

LRMS (electrospray): m/z [M+Na]⁺ 518, [M−H]⁺ 494.

HRMS: Found [M+H]⁺ 496.1685. C₂₆H₂₇FN₃O₄S requires 496.1701 Found [M+Na]⁺ 518.1504. C₂₆H₂₆FN₃O₄SNa requires 518.1520.

### Example 38

### 5-Fluoro-N-[1-(2-hydroxy-4-methoxy-benzoyl)-piperidin-4-yl]-2-(3-methylsulfanyl-phenoxy)-nicotinamide

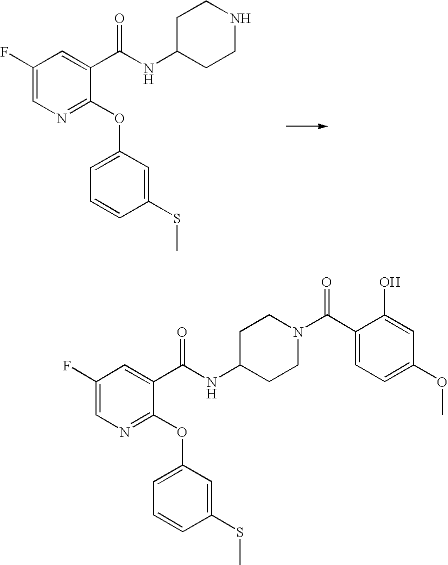

2-Hydroxy-4-methoxy-benzoic acid (75 mg, 0.45 mmol) was added to a solution of 5-fluoro-2-(3-methylsulfanyl-phenoxy)-N-piperidin-4-yl-nicotinamide (180 mg, 0.499 mmol), 1-hydroxybenzotriazole (71 mg, 0.523 mmol) and N-methylmorpholine (219 μl, 2.0 mmol) in dichloromethane (2 ml) under nitrogen at room temperature. 1-(3-Dimethylaminopropyl)-3-ethylcarbodiimide hydrochloride (143 mg, 0.746 mmol) was then added and the reaction was stirred at room temperature for 20 h. After quenching with sat. ammonium chloride (1 ml) and diluting with water (3 ml), the organic phase was collected via a hydrophobic separation cartridge, concentrated under reduced pressure and the residue was purified by flash column chromatography on a biotage system eluting with a solvent gradient of ethyl acetate:cyclohexane (15:85 changing to 80:20, by volume) to give 5-fluoro-N-[1-(2-hydroxy-4-methoxy-benzoyl)-piperidin-4-yl]-2-(3-methylsulfanyl-phenoxy)-nicotinamide (84 mg) as an off-white foam.

¹H NMR (400 MHz, CD₃OD): δ=8.10-8.13 (1H, d), 7.97-8.00 (1H, dd), 7.29-7.35 (2H, t), 7.08-7.13 (1H, t), 7.04-7.06 (1H, m), 6.87-6.91 (1H, d), 6.44-6.48 (1H, d), 6.38-6.40 (1H, d), 4.00-4.20 (2H, brs+m), 3.78 (3H, s), 3.12-3.21 (2H, t), 2.44 (3H, s), 1.97-2.04 (2H, m), 1.56-1.66 (2H, m), 1.26-1.30 (2H, brs) ppm.

LRMS (electrospray): m/z [M+Na]⁺ 534, [M−H]⁺ 510.

HRMS: Found [M+H]⁺ 512.1634. C₂₆H₂₇FN₃O₅S requires 512.1650.

### Example 39

### 5-Fluoro-N-[1-(2-hydroxy-benzoyl)-piperidin-4-yl]-2-(3-methylsulfanyl-phenoxy)-nicotinamide

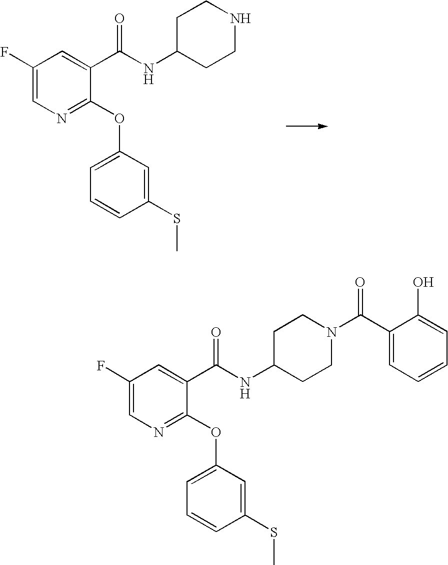

2-Hydroxy-benzoic acid (62 mg, 0.45 mmol) was added to a solution of 5-fluoro-2-(3-methylsulfanyl-phenoxy)-N-piperidin-4-yl-nicotinamide (180 mg, 0.499 mmol), 1-hydroxybenzotriazole (71 mg, 0.523 mmol) and N-methylmorpholine (219 μl, 2.0 mmol) in dichloromethane (2 ml) under nitrogen at room temperature. 1-(3-Dimethylaminopropyl)-3-ethylcarbodiimide hydrochloride (143 mg, 0.746 mmol) was then added and the reaction was stirred at room temperature for 20 h. After quenching with sat. ammonium chloride (1 ml) and diluting with water (3 ml), the organic phase was collected via a hydrophobic separation cartridge, concentrated under reduced pressure and the residue was purified by flash column chromatography on a biotage system eluting with a solvent gradient of ethyl acetate:cyclohexane (15:85 changing to 80:20, by volume) to give 5-fluoro-N-[1-(2-hydroxy-benzoyl)-piperidin-4-yl]-2-(3-methylsulfanyl-phenoxy)-nicotinamide (72 mg) as an off-white foam.

¹H NMR (400 MHz, CD₃OD): δ=8.10-8.13 (1H, d), 7.97-8.01 (1H, dd), 7.30-7.35 (1H, t), 7.22-7.27 (1H, t), 7.11-7.18 (2H, m), 7.06 (1H, s), 6.82-6.90 (3H, m), 4.13-4.20 (1H, m), 3.13-3.22 (2H, t), 2.45 (3H, s), 1.95-2.05 (2H, m), 1.57-1.68 (2H, m), ppm.

LRMS (electrospray): m/z [M+Na]⁺ 504, [M−H]⁺ 480.

HRMS: Found [M+H]⁺ 482.1529. C₂₅H₂₅FN₃O₄S requires 482.1545 Found [M+Na]⁺ 504.1349. C₂₅H₂₄FN₃O₄SNa requires 504.1364.

### Example 40

### N-(1-Acetyl-piperidin-4-yl)-2-(4-ethylsulfanyl-phenoxy)-5-fluoro-nicotinamide

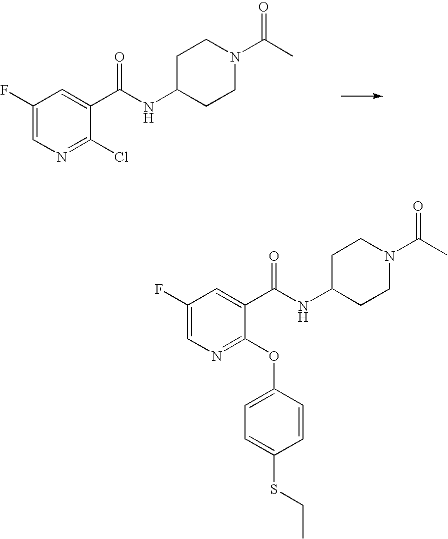

4-ethylsulfanyl-phenol (79 mg, 0.51 mmol) was added to a solution of N-(1-Acetyl-piperidin-4-yl)-2-chloro-5-fluoro-nicotinamide (140 mg, 0.47 mmol) and caesium carbonate (213 mg, 0.65 mmol) in toluene:N-methylpyrrolidine (4:1, 2 ml) under nitrogen at room temperature. Copper(I)iodide (9 mg, 0.05 mmol) was then added and the reaction was heated to 110° C. and stirred at this temperature under nitrogen for 2 h. The reaction mixture was then cooled, filtered through arbocel and the filtrate was diluted with ethyl acetate (15 ml). The mixture was washed with water (15 ml), dried over Mg₂SO₄ and concentrated under reduced pressure. The residue was purified by flash column chromatography on silica gel eluting with a solvent gradient of dichloromethane:methanol:concentrated aqueous ammonia (99.5:0.5:0.05 changing to 95:5:0.5, by volume). The product was then dissolved in ethyl acetate (15 ml), washed with water (15 ml), dried over Mg₂SO₄ and concentrated under reduced pressure to give N-(1-Acetyl-piperidin-4-yl)-2-4-ethylsulfanyl-phenoxy)-5-fluoro-nicotinamide (51 mg) as an off-white foam.

¹H NMR (400 MHz, CDCl₃): δ=8.31-8.38 (1H, dd), 8.03-8.05 (1H, d), 7.78-7.86 (1H, d), 7.37-7.43 (2H, d), 7.00-7.09 (2H, d), 4.39-4.48 (1H, d), 4.19-4.29 (1H, m), 3.71-3.79 (1H, d), 3.20-3.29 (1H, t), 2.83-2.99 (3H, quart+t), 2.00-2.20 (5H, m+s), 1.39-1.53 (2H, m), 1.33-1.39 (3H, t) ppm.

LRMS (electrospray): m/z [M+Na]⁺ 440, [M−H]⁺ 417.

Anal. Found C, 59.06; H, 5.82; N, 9.57. C₂₁H₂₄FN₃O₃S. 0.1 mol H₂O requires C, 59.24; H, 5.75; N, 9.82%.

### Example 41

### N-(1-Acetyl-piperidin-4-yl)-2-(4-methylsulfanyl-phenoxy)-5-fluoro-nicotinamide

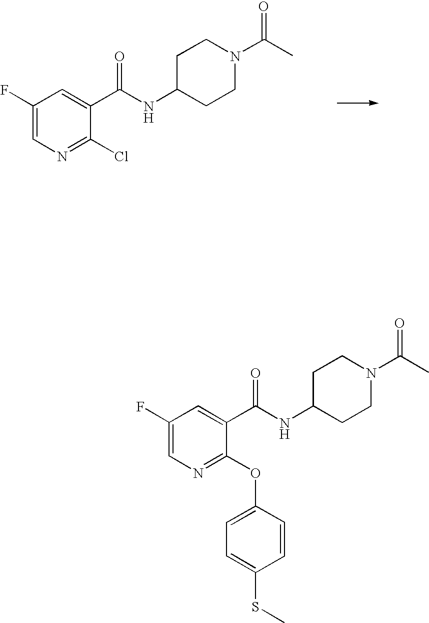

N-(1-Acetyl-piperidin-4-yl)-2-chloro-5-fluoro-nicotinamide (100 mg, 0.33 mmol), 4-methylsulfanyl-phenol (51 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to N-(1-acetyl-piperidin-4-yl)-2-(4-methylsulfanyl-phenoxy)-5-fluoro-nicotinamide (27 mg) as an off-white solid.

¹H NMR (400 MHz, CDCl₃): δ=8.31-8.36 (1H, dd), 8.01-8.03 (1H, d), 7.80-7.86 (1H, d), 7.30-7.34 (2H, d), 7.02-7.06 (2H, d), 4.37-4.44 (1H, d), 4.18-4.28 (1H, m), 3.72-3.79 (1H, d), 3.19-3.28 (1H, t), 2.83-2.95 (1H, t), 2.48 (3H, s), 2.00-2.17 (5H, 2xm+s), 1.39-1.50 (2H, m) ppm.

LRMS (electrospray): m/z [M+Na]⁺ 426, [M−H]⁺ 402.

HRMS: Found [M+H]⁺ 404.1446. C₂₀H₂₃FN₃O₃S requires 404.1439 Found [M+Na]⁺ 426.1265. C₂₀H₂₂FN₂O₃SNa requires 426.1258.

### Example 42

### N-(1-Acetyl-piperidin-4-yl)-2-(4-methylsulfanyl-3-methyl-phenoxy)-5-fluoro-nicotinamide

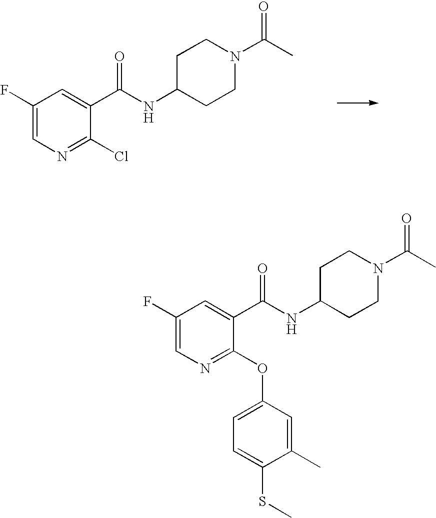

N-(1-Acetyl-piperidin-4-yl)-2-chloro-5-fluoro-nicotinamide (100 mg, 0.33 mmol), 4-methylsulfanyl-3-methyl-phenol (57 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(4-methylsulfanyl-3-methyl-phenoxy)-5-fluoro-nicotinamide (34 mg) as an off-white solid.

¹H NMR (400 MHz, CDCl₃): δ=8.29-8.35 (1H, dd), 8.02-8.04 (1H, d), 7.83-7.89 (1H, d), 7.21-7.24 (1H, d), 6.92-6.96 (2H, d+s), 4.37-4.44 (1H, d), 4.19-4.28 (1H, m), 3.69-3.78 (1H, d), 3.20-3.28 (1H, t), 2.84-2.93 (1H, t), 2.46 (3H, s), 2.37 (3H, s), 2.00-2.17 (5H, 2xm+s), 1.40-1.50 (2H, m) ppm.

LRMS (electrospray): m/z [M+Na]⁺ 440, [M−H]⁺ 416.

HRMS: Found [M+H]⁺ 418.1603 C₂₁H₂₅FN₃O₃S requires 418.1595.

### Example 43

### N-(1-Acetyl-piperidin-4-yl)-2-(3-methylsulfanyl-4-chloro-phenoxy)-5-fluoro-nicotinamide

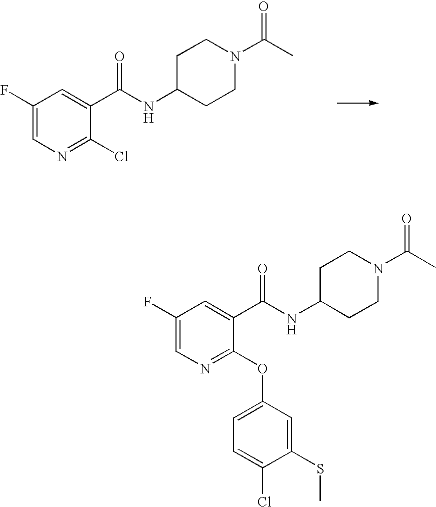

N-(1-Acetyl-piperidin-4-yl)-2-chloro-5-fluoro-nicotinamide (100 mg, 0.33 mmol), 3-methylsulfanyl-4-chloro-phenol (U.S. Pat. No. 4,005,148) (64 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(3-methylsulfanyl-4-chloro-phenoxy)-5-fluoro-nicotinamide (21 mg) as an off-white solid.

¹H NMR (400 MHz, CDCl₃): δ=8.31-8.36 (1H, dd), 8.02-8.04 (1H, d), 7.69-7.74 (1H, d), 7.37-7.40 (1H, d), 6.92 (1H, s), 6.79-6.83 (1H, d), 4.42-4.49 (1H, d), 4.18-4.29 (1H, m), 3.74-3.81 (1H, d), 3.20-3.28 (1H, t), 2.82-2.90 (1H, t), 2.44 (3H, s), 2.00-2.19 (5H, d+s), 1.39-1.51 (2H, m) ppm.

LRMS (electrospray): m/z [M+Na]⁺ 460, 462, [M−H]⁺ 436, 438.

HRMS: Found [M+H]⁺ 438.1055. C₂₀H₂₂ClFN₃O₃S requires 438.1049 Found [M+Na]⁺ 460.0868. C₂₀H₂₁ClFN₃O₃SNa requires 460.0868.

### Example 44

### N-(1-Acetyl-piperidin-4-yl)-2-(4-methylsulfanyl -3-chloro-phenoxy)-5-fluoro-nicotinamide

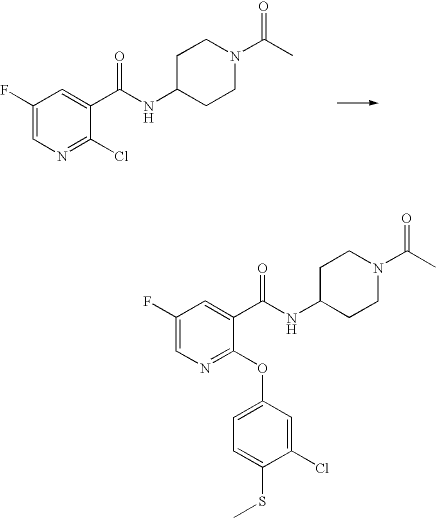

N-(1-Acetyl-piperidin-4-yl)-2-chloro-5-fluoro-nicotinamide (100 mg, 0.33 mmol), 3-methylsulfanyl-4-chloro-phenol (64 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(4-methylsulfanyl-3-chloro-phenoxy)-5-fluoro-nicotinamide (4.6 mg) as an off-white solid.

¹H NMR (400 MHz, CDCl₃): δ=8.31-8.36 (1H, dd), 8.03-8.05 (1H, d), 7.67-7.74 (1H, d), 7.24 (1H, s), 7.18-7.20 (1H, d), 7.02-7.05 (1H, dd), 4.40-4.49 (1H, d), 4.18-4.29 (1H, m), 3.72-3.79 (1H, d), 3.20-3.28 (1H, t), 2.82-2.93 (1H, t), 2.49 (3H, s), 2.00-2.19 (5H, m+s), 1.39-1.53 (2H, m) ppm.

LRMS (electrospray): m/z [M+Na]⁺ 460, 462, [M−H]⁺ 436, 438.

HRMS: Found [M+H]⁺ 438.1056. C₂₀H₂₂ClFN₃O₃S requires 438.1049 Found [M+Na]⁺ 460.0874. C₂₀H₂₁ClFN₃O₃SNa requires 460.0868.

### Example 45

### N-(1-Acetyl-piperidin-4-yl)-2-(4-methylsulfanyl-3,5-dimethyl-phenoxy)-5-fluoro-nicotinamide

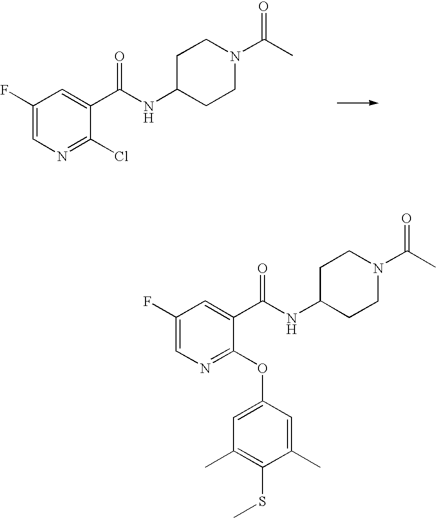

N-(1-Acetyl-piperidin-4-yl)-2-chloro-5-fluoro-nicotinamide (100 mg, 0.33 mmol), 4-methylsulfanyl-3,5-dimethyl-phenol (62 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(4-methylsulfanyl-3,5-dimethyl-phenoxy)-5-fluoro-nicotinamide (24 mg) as an off-white solid.

¹H NMR (400 MHz, CDCl₃): δ=8.28-8.35 (1H, dd), 8.03-8.05 (1H, d), 7.77-7.84 (1H, m), 6.83 (2H, s), 4.37-4.46 (1H, d), 4.17-4.28 (1H, m), 3.70-3.79 (1H, d), 3.18-3.28 (1H, t), 2.81-2.93 (1H, t), 2.58 (6H, s), 2.23 (3H, s), 2.00-2.19 (5H, 2xm+s), 1.38-1.50 (2H, m) ppm.

LRMS (electrospray): m/z [M+Na]⁺ 454, [M−H]⁺ 430.

HRMS: Found [M+H]⁺ 432.1757. C₂₂H₂₇FN₃O₃S requires 432.1752 Found [M+Na]⁺ 454.1580. C₂₂H₂₆FN₃O₃SNa requires 454.1571.

### Example 46

### N-(1-Acetyl-piperidin-4-yl)-2-(2,3-dihydrobenzo[b]thiophen-5-yloxy)-5-fluoro-nicotinamide

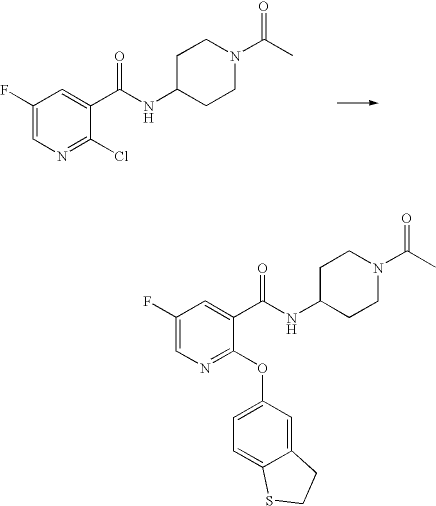

N-(1-Acetyl-piperidin-4-yl)-2-chloro-5-fluoro-nicotinamide (100 mg, 0.33 mmol), 2,3-dihydrobenzo[b]thiophen-5-ol (56 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(2,3-dihydrobenzo[b]thiophen-5-yloxy)-5-fluoro-nicotinamide (34 mg) as an off-white solid.

¹H NMR (400 MHz, CDCl₃): δ=8.28-8.35 (1H, dd), 8.02-8.04 (1H, d), 7.80-7.94 (1H, d), 7.22-7.26 (1H, d), 6.93 (1H, s), 6.83-6.88 (1H, d), 4.38-4.48 (1H, d), 4.18-4.29 (1H, m), 3.71-3.80 (1H, d), 3.38-3.45 (2H, m), 3.19-3.37 (3H, m), 2.84-2.97 (1H, t), 1.90-2.20 (5H, m+s), 1.39-1.55 (2H, m) ppm.

LRMS (electrospray): m/z [M+Na]⁺ 438, [M−H]⁺ 414.

HRMS: Found [M+H]⁺ 416.1445. C₂₁ H₂₃FN₃O₃S requires 416.1439 Found [M+Na]⁺ 438.1269. C₂₁H₂₂FN₃O₃SNa requires 438.1258.

### Example 47

### N-(1-Acetyl-piperidin-4-yl)-2-(2,3-dihydrobenzo[1,4]oxathiin-7-yloxy)-5-fluoro-nicotinamide

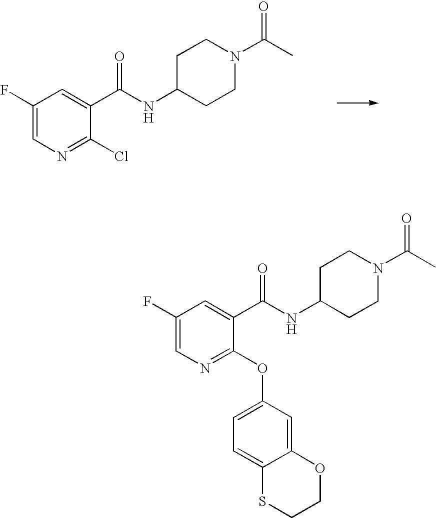

N-(1-Acetyl-piperidin-4-yl)-2-chloro-5-fluoro-nicotinamide (100 mg, 0.33 mmol), 2,3-dihydrobenzo[1,4]oxathiin-7-ol (62 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(2,3-dihydrobenzo[1,4]oxathiin-7-yloxy)-5-fluoro-nicotinamide (33 mg) as an off-white solid.

¹H NMR (400 MHz, CDCl₃): δ=8.30-8.35 (1H, dd), 8.04-8.06 (1H, d), 7.78-7.87 (1H, d), 7.05-7.09 (1H, d), 6.62-6.66 (2H, s+d), 4.36-4.47 (3H, 2xm), 4.18-4.29 (1H, m), 3.72-3.79 (1H, d), 3.20-3.30 (1H, t), 3.12-3.17 (2H, m), 2.86-2.98 (1H, t), 1.99-2.17 (5H, 2xm+s), 1.39-1.53 (2H, m) ppm.

LRMS (electrospray): m/z [M+Na]⁺ 454, [M−H]⁺ 430.

HRMS: Found [M+H]⁺ 432.1398. C₂₁ H₂₃FN₃O₄S requires 432.1388 Found [M+Na]⁺ 454.1215. C₂₁ H₂₂FN₃O₄SNa requires 454.1207.

### Example 48

### N-(1-Acetyl-piperidin-4-yl)-2-(4-methylsulfanyl-2-fluoro-phenoxy)-5-fluoro-nicotinamide

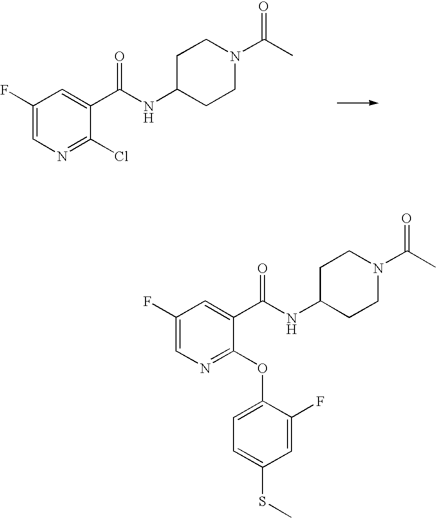

N-(1-Acetyl-piperidin-4-yl)-2-chloro-5-fluoro-nicotinamide (100 mg, 0.33 mmol), 4-methylsulfanyl-2-fluoro-phenol (58 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(4-methylsulfanyl-2-fluoro-phenoxy)-5-fluoro-nicotinamide (12 mg) as an off-white solid.

¹H NMR (400 MHz, CDCl₃): δ=8.30-8.35 (1H, dd), 8.00-8.02 (1H, d), 7.73-7.79 (1H, d), 7.22-7.25 (1H, d), 7.08-7.13 (2H, m), 4.38-4.47 (1H, d), 4.20-4.30 (1H, m), 3.74-3.80 (1H, d), 3.20-3.29 (1H, t), 2.87-2.97 (1H, t), 2.51 (3H, s), 2.00-2.19 (5H, 2xm+s), 1.41-1.55 (2H, m) ppm.

LRMS (electrospray): m/z [M+Na]⁺ 444, [M−H]⁺ 420.

HRMS: Found [M+H]⁺ 422.1350. C₂₀H₂₂F₂N₃O₃S requires 422.1345 Found [M+Na]⁺ 444.1172. C₂₀H₂₁F₂N₃O₃SNa requires 444.1164.

### Example 49

### N-(1-Acetyl-piperidin-4-yl)-2-(3-methylsulfanyl-4-methyl-phenoxy)-5-fluoro-nicotinamide

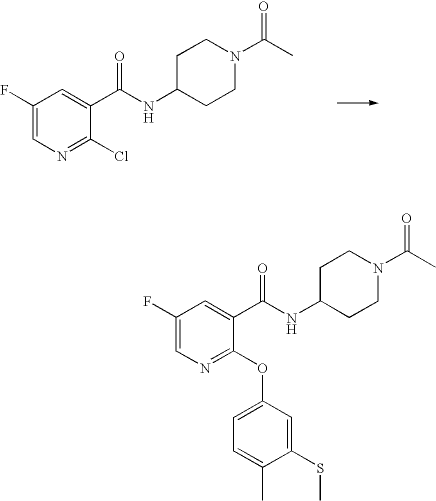

N-(1-Acetyl-piperidin-4-yl)-2-chloro-5-fluoro-nicotinamide (100 mg, 0.33 mmol), 3-methylsulfanyl-4-methyl-phenol (57 mg, 0.37 mmol) and caesium carbonate (163 mg, 0.5 mmol) were suspended in dimethylformamide (1.5 ml) and the reaction was heated to 55° C. and stirred at this temperature under nitrogen for 18 h. The reaction was quenched with sat. ammonium chloride solution (1 ml) and water (1 ml) and the organic phase was collected by passing the mixture through a chemelute cartridge, washing with ethyl acetate (20 ml). The solvent was removed on a Genevac and the residue was purified by preparative HPLC to give N-(1-acetyl-piperidin-4-yl)-2-(3-methylsulfanyl-4-methyl-phenoxy)-5-fluoro-nicotinamide (24 mg) as an off-white solid.

¹H NMR (400 MHz, CDCl₃): δ=8.32-8.36 (1H, dd), 8.02-8.04 (1H, d), 7.83-7.89 (1H, d), 7.17-7.21 (1H, d), 6.87-6.89 (1H, d), 6.77-6.81 (1H, dd), 4.39-4.47 (1H, d), 4.19-4.29 (1H, m), 3.73-3.80 (1H, d), 3.20-3.28 (1H, t), 2.84-2.94 (1H, t), 2.43 (3H, s), 2.34 (3H, s), 2.00-2.18 (5H, s+m), 1.39-1.53 (2H, m) ppm.

LRMS (electrospray): m/z [M+Na]⁺ 440, [M−H]⁺ 416.

HRMS: Found [M+H]⁺ 418.1593. C₂₁H₂₅FN₃O₃S requires 418.1595.

### Example 50

### 5-Fluoro-N-(1-methanesulfonyl-piperidin-4-yl)-2-(3-methylsulfanyl-phenoxy)-nicotinamide

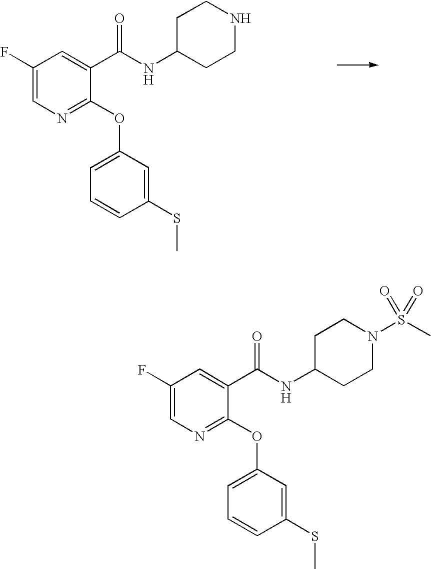

5-Fluoro-2-(3-methylsulfanyl-phenoxy)-N-piperidin-4-yl-nicotinamide (107 mg, 0.269 mmol) was suspended in dichloromethane (4 ml) under nitrogen at −78° C. and triethylamine (83□l, 0.538 mmol) was added followed by methanesulfonyl chloride (35□l, 0.403 mmol). The reaction was allowed to warm to room temperature for 2 h and was quenched with sat. sodium bicarbonate solution (1 ml). After diluting with water (4 ml) the aqueous phase was extractred with dichloromethane (10 ml) and the organic phase was removed and concentrated under reduced pressure. The residue was purified by flash column chromatography on silica gel eluting with ethyl acetate:cyclohexane (40:60 changing to 90:10, by volume) to give 5-fluoro-N-(1-methanesulfonyl-piperidin-4-yl)-2-(3-methylsulfanyl-phenoxy)-nicotinamide (99 mg) as a white foam.

¹H NMR (400 MHz, CD₃OD): δ=8.08-8.10 (1H, d), 7.97-8.00 (1H, m), 7.28-7.33 (1H, t), 7.10-7.14 (1H, d), 7.03 (1H, s), 6.84-6.89 (1H, d), 3.98-4.06 (1H, m), 3.58-3.65 (2H, m), 2.93-3.00 (2H, t), 2.78 (3H, s), 2.43 (3H, s), 2.00-2.08 (2H, m), 1.61-1.73 (2H, m) ppm.

LRMS (electrospray): m/z [M+Na]⁺ 462, [M−H]⁺ 438.

Anal. Found C, 50.91; H, 4.96; N, 8.99. C₁₉H₂₂FN₃O₄S₂. 0.6 mol H₂O requires 50.68; H, 5.19; N, 9.33%.

### Example 51

### N-(2-Ethyl-2H -pyrazol -3-yl)-5-fluoro-2-(3-methylsulfanyl-phenoxy)-nicotinamide

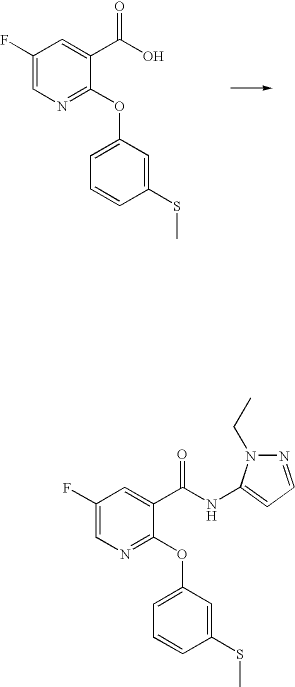

1-(3-Dimethylaminopropyl)-3-ethylcarbodiimide hydrochloride (75 mg, 0.394 mmol) and 1-hydroxybenzotriazole (60 mg, 0.394 mmol) were added to a suspension of 5-fluoro-2-(3-methylsulfanyl-phenoxy)-nicotinic acid (100 mg, 0.358 mmol) in dichloromethane (2 ml). This was stirred under nitrogen at room temperature for 15 min, and a solution of 2-ethyl-2H-pyrazol-3-ylamine (44 mg, 0.394 mmol) in dichloromethane (2 ml) was added. The reaction was stirred under nitrogen at room temperature for 72 h and the mixture was quenched with water (1 ml) and diluted with dichloromethane (5 ml). The mixture was then passed through a phase separation cartridge and the organic layer was concentrated under reduced pressure. The residue was purified by flash column chromatography on silica gel eluting with ethyl acetate:cyclohexane (1:2, by volume) to give N-(2-Ethyl-2H-pyrazol-3-yl)-5-fluoro-2-(3-methylsulfanyl-phenoxy)-nicotinamide (53 mg) as a crystalline white solid.

¹H NMR (400 MHz, CDCl₃): δ=9.82 (1H, s), 8.40-8.44 (1H, dd), 8.13-8.16 (1H, d), 7.46-7.47 (1H, d), 7.37-7.41 (1H, t), 7.18-7.22 (1H, d), 7.05-7.08 (1H, t), 6.91-6.96 (1H, m), 6.57 (1H, s), 4.024.10 (2H, quart), 2.50 (3H, s), 1.34-1.40 (3H, t) ppm.

LRMS (electrospray): m/z [M+H]⁺ 373, [M+Na]⁺ 395, [M−H]⁺ 371.

### Example 52

### 5-Fluoro-N-(3-hydroxy-pyridin-2-yl)-2-(3-methylsulfanyl-phenoxy)-nicotinamide

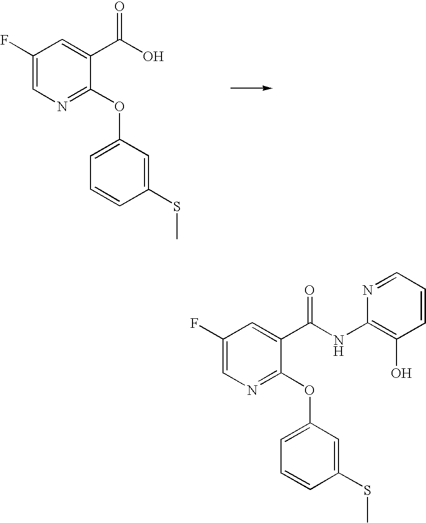

2-Amino-3-hydroxypyridine (83 mg, 0.752 mmol) and triethylamine (300□l, 2.14 mmol) were dissolved in dimethylformamide (800□l) under nitrogen at room temperature and a solution of 5-fluoro-2-(3-methylsulfanyl-phenoxy)-nicotinic acid (150 mg, 0.537 mmol), 1-(3-dimethylaminopropyl)-3-ethylcarbodiimide hydrochloride (113 mg, 0.590 mmol) and 1-hydroxybenzotriazole (80 mg, 0.590 mmol) in dimethylformamide (5 ml) and the reaction was stirred at room temperature for 24 h. The reaction mixture was concentrated under reduced pressure, and the residue was partitioned between ethyl acetate (20 ml) and 1M HCl (20 ml). The organic phase was removed, washed with water (20 ml) and the solvent was removed under reduced pressure. The residue was purified by flash column chromatography on silica gel eluting with dichloromethane:methanol:concentrated aqueous ammonia (95:5:0.5, by volume) and the product was crystallised from DIISOPROPYLETHER (5 ml) to give 5-fluoro-N-(3-hydroxy-pyridin-2-yl)-2-(3-methylsulfanyl-phenoxy)-nicotinamide (87 mg) as a white solid.

¹H NMR (400 MHz, CDCl₃): δ=10.42-10.50 (1H, brs), 10.13-10.18 (1H, brs), 8.38-8.42 (1H, m), 8.27-8.31 (1H, m), 8.12-8.16 (1H, m), 7.33-7.39 (2H, m), 7.14-7.18 (1H, d), 7.10-7.14 (2H, m), 6.95-7.00 (1H, d), 2.48 (3H, s) ppm.

LRMS (electrospray): m/z [M+H]⁺ 372, [M+Na]⁺ 394, [M−H]⁺ 370.

Anal. Found C, 57.62; H, 3.70; N, 11.11. C₁₈H₁₄FN₃O₃S. 0.25 mol H₂O requires C, 57.51; H, 3.88; N, 11.18%.

### Example 53

### 5-Fluoro-2-(3-methylsulfanyl-phenoxy)-N-pyridin-4-yl-nicotinamide

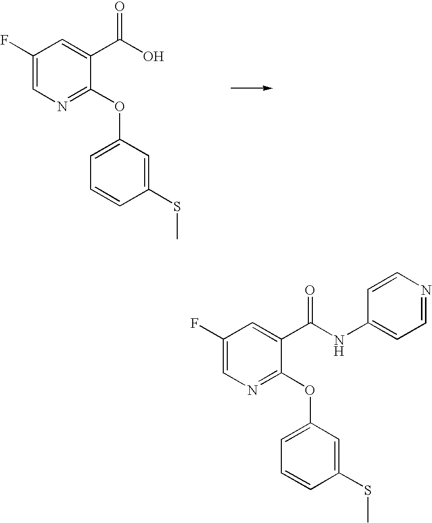

4-Aminopyridine (120 mg, 1.28 mmol) and triethylamine (355□l, 2.56 mmol) were dissolved in dimethylformamide (1 ml) under nitrogen at room temperature and a solution of 5-fluoro-2-(3-methylsulfanyl-phenoxy)-nicotinic acid (355 mg, 1.28 mmol), 1-(3-dimethylaminopropyl)-3-ethylcarbodiimide hydrochloride (268 mg, 1.41 mmol) and 1-hydroxybenzotriazole (189 mg, 1.41 mmol) in dimethylformamide (7.5 ml) and the reaction was stirred at room temperature for 24 h. The reaction mixture was concentrated under reduced pressure, and the residue was partitioned between ethyl acetate (20 ml) and 1M HCl (20 ml). The organic phase was removed, washed with 10% sodium bicarbonate solution (20 ml), brine (20 ml), dried over Na₂SO₄ and the solvent was removed under reduced pressure. The residue was purified by flash column chromatography on silica gel eluting with dichloromethane:methanol:concentrated aqueous ammonia (97.5:2.5:0.25, by volume) and the product was crystallised from DIISOPROPYLETHER (5 ml) to give 5-fluoro-2-(3-methylsulfanyl-phenoxy)-N-pyridin-4-yl-nicotinamide (87 mg) as a white solid.

¹H NMR (400 MHz, CDCl₃): δ=9.84-9.96 (1H, brs), 8.54-8.60 (2H, m), 8.40-8.46 (1H, dd), 8.13-8.16 (1H, d), 7.58-7.63 (2H, d), 7.38-7.44 (1H, t), 7.20-7.24 (1H, d), 7.10 (1H, s), 6.94-7.00 (1H, d), 2.50 (3H, s) ppm.

LRMS (electrospray): m/z [M+H]⁺ 356, [M+Na]⁺ 378, [M−H]⁺ 354.

Anal. Found C, 60.56; H, 3.91; N, 11.69. C₁₈H₁₄FN₃O₃S requires C, 60.83; H, 3.97; N, 11.82%.

### Example 54

### 5-Fluoro-2-(3-methylsulfanyl-phenoxy)-N-pyridin-2-yl-nicotinamide

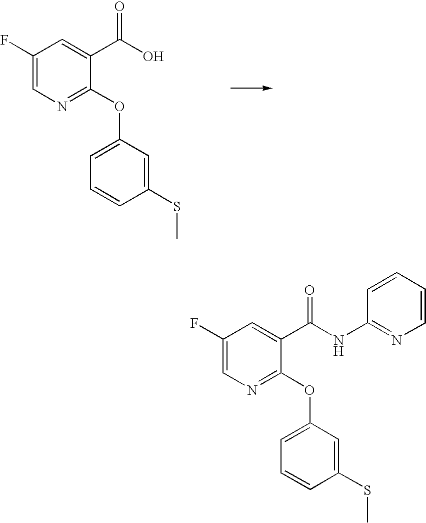

2-Aminopyridine (120 mg, 1.28 mmol) and triethylamine (355□l, 2.56 mmol) were dissolved in dimethylformamide (1 ml) under nitrogen at room temperature and a solution of 5-fluoro-2-(3-methylsulfanyl-phenoxy)-nicotinic acid (355 mg, 1.28 mmol), 1-(3-dimethylaminopropyl)-3-ethylcarbodiimide hydrochloride (268 mg, 1.41 mmol) and 1-hydroxybenzotriazole (189 mg, 1.41 mmol) in dimethylformamide (7.5 ml) and the reaction was stirred at room temperature for 24 h. The reaction mixture was concentrated under reduced pressure, and the residue was partitioned between ethyl acetate (20 ml) and 1M HCl (20 ml). The organic phase was removed, washed with 10% sodium bicarbonate solution (20 ml), brine (20 ml), dried over Na₂SO₄ and the solvent was removed under reduced pressure. The residue was purified by flash column chromatography on silica gel eluting with dichloromethane:methanol:concentrated aqueous ammonia (97.5:2.5:0.25, by volume) and the product was crystallised from DIISOPROPYLETHER (5 ml) to give 5-fluoro-2-(3-methylsulfanyl-phenoxy)-N-pyridin-4-yl-nicotinamide (89 mg) as a white solid.

¹H NMR (400 MHz, CDCl₃): δ=10.20-10.29 (1H, brs), 8.38-8.46 (2H, dm), 8.31-8.36 (1H, m), 8.13-8.15 (1H, d), 7.75-7.80 (1H, m), 7.35-7.40 (1H, t), 7.17-7.21 (1H, d), 7.06-7.16 (2H, m), 6.98-7.02 (1H, dd), 2.47 (3H, s) ppm.

LRMS (electrospray): mlz [M+Na]⁺ 378, [2M+Na]⁺ 733, [M−H]⁺ 354.

Anal. Found C, 60.34; H, 3.96; N, 11.59. C₁₈H₁₄FN₃O₃S requires C, 60.83; H, 3.97; N, 11.83%.

### Example 55

### 5-Fluoro-2-(3-methylsulfanyl-phenoxy)-N-pyridin-3-yl-nicotinamide

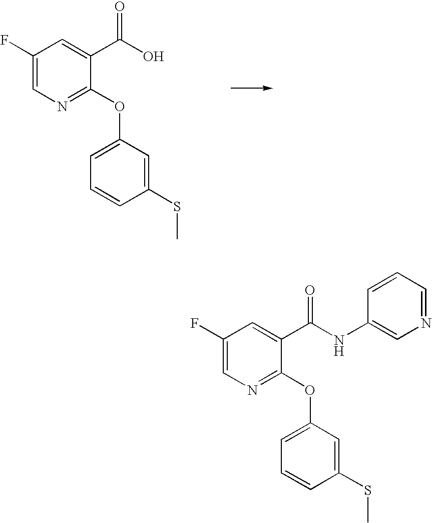

3-Aminopyridine (120 mg, 1.28 mmol) and triethylamine (355□l, 2.56 mmol) were dissolved in dimethylformamide (1 ml) under nitrogen at room temperature and a solution of 5-fluoro-2-(3-methylsulfanyl-phenoxy)-nicotinic acid (355 mg, 1.28 mmol), 1-(3-dimethylaminopropyl)-3-ethylcarbodiimide hydrochloride (268 mg, 1.41 mmol) and 1-hydroxybenzotriazole (189 mg, 1.41 mmol) in dimethylformamide (7.5 ml) and the reaction was stirred at room temperature for 24 h. The reaction mixture was concentrated under reduced pressure, and the residue was partitioned between ethyl acetate (20 ml) and 1M HCl (20 ml). The organic phase was removed, washed with 10% aqueous ammonia solution (20 ml), brine (20 ml), dried over Na₂SO₄ and the solvent was removed under reduced pressure. The residue was purified by flash column chromatography on silica gel eluting with dichloromethane:methanol:concentrated aqueous ammonia (97.5:2.5:0.25, by volume) and the product was crystallised from DIISOPROPYLETHER (5 ml) to give 5-fluoro-2-(3-methylsulfanyl-phenoxy)-N-pyridin-3-yl-nicotinamide (197 mg) as a white solid.

¹H NMR (400 MHz, CDCl₃): δ=9.77-9.84 (1H, brs), 8.61-8.74 (1H, d), 8.39-8.49 (2H, dd), 8.26-8.32 (1H, d), 8.14 (1H, s), 7.38-7.44 (1H, t), 7.29-7.37 (1H, m), 7.19-7.24 (1H, d), 7.11 (1H, s), 6.95-7.02 (1H, d), 2.50 (3H, s) ppm.

LRMS (electrospray): m/z [M+Na]⁺ 378, [2M+Na]⁺ 733, [M−H]⁺ 354.

Anal. Found C, 60.33; H, 3.89; N, 11.66. C₁₈H₁₄FN₃O₃S. 0.15 mol H₂O requires C, 60.38; H, 4.03; N, 11.73%.

### Example 56

### 5-Fluoro-N-(4-hydroxy-pyrimidin-2-yl-)2-(3-methylsulfanyl-phenoxy)-nicotinamide

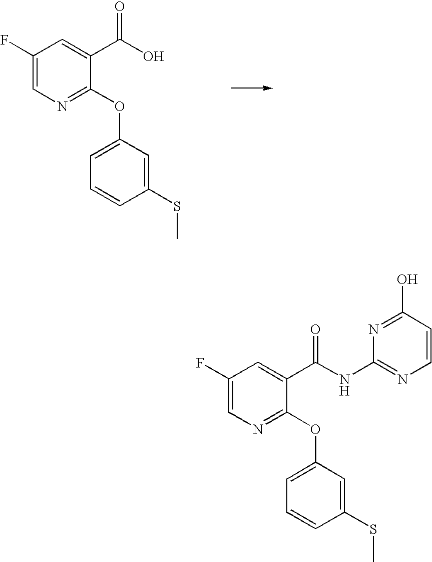

2-Amino-4-hydroxypyrimidine (142 mg, 1.28 mmol) and triethylamine (355□l, 2.56 mmol) were dissolved in dimethylformamide (1 ml) under nitrogen at room temperature and a solution of 5-fluoro-2-(3-methylsulfanyl-phenoxy)-nicotinic acid (355 mg, 1.28 mmol), 1-(3-dimethylaminopropyl)-3-ethylcarbodiimide hydrochloride (268 mg, 1.41 mmol) and 1-hydroxybenzotriazole (189 mg, 1.41 mmol) in dimethylformamide (7.5 ml) and the reaction was stirred at room temperature for 24 h. The reaction mixture was concentrated under reduced pressure, and the residue was partitioned between dichloromethane (20 ml) and water (20 ml). The organic phase was dried over Na₂SO₄ and the solvent was removed under reduced pressure. The residue was purified by flash column chromatography on silica gel eluting with dichloromethane:methanol:concentrated aqueous ammonia (97.5:2.5:0.25, by volume) and the product was crystallised from DIISOPROPYLETHER (5 ml) to give 5-fluoro-N-(4-hydroxy-pyrimidin-2-yl-)2-(3-methylsulfanyl-phenoxy)-nicotinamide (7 mg) as a white solid.

¹H NMR (400 MHz, CDCl₃): δ=8.38-8.41 (1H, dd), 8.09-8.11 (1H, d), 7.72-7.77 (1H, d), 7.36-7.41 (1H, t), 7.23-7.26 (2H, m, partially masked by solvent), 7.19-7.22 (1H, dd), 7.07 (1H, s), 6.94-6.98 (1H, dd), 6.19-6.23 (1H, d), 2.48 (3H, s) ppm.

LRMS (electrospray): m/z [M+Na]⁺ 395, [2M+Na]⁺ 767, [M−H]⁺ 371.

### Example 57

### 5-Fluoro-N-(6-methoxy-pyridin-3-yl)-2-(3-methylsulfanyl-phenoxy)-nicotinamide

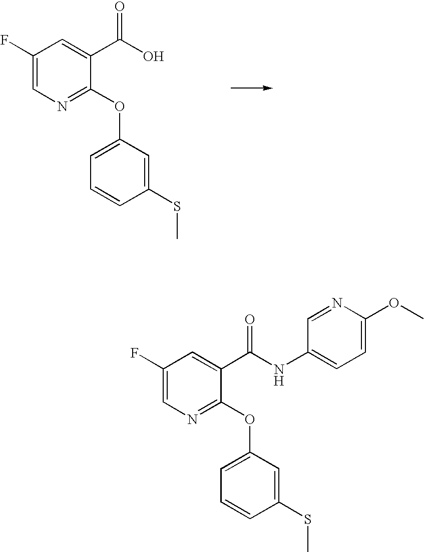

5-Amino-2-methoxypyridine (89 mg, 0.716 mmol) and triethylamine (200□l, 2.14 mmol) were dissolved in dimethylformamide (1 ml) under nitrogen at room temperature and a solution of 5-Fluoro-2-(3-methylsulfanyl-phenoxy)-nicotinic acid (200 mg, 0.716 mmol), 1-(3-dimethylaminopropyl)-3-ethylcarbodiimide hydrochloride (151 mg, 0.788 mmol) and 1-hydroxybenzotriazole (106 mg, 0.788 mmol) in dimethylformamide (5 ml) and the reaction was stirred at room temperature for 18 h. The reaction mixture was concentrated under reduced pressure, and the residue was purified by flash column chromatography on silica gel eluting with a solvent gradient of dichloromethane:methanol:concentrated aqueous ammonia (100:0:0 changing to 98:2:0 then 97.5:2.5 0.25, by volume) and the product was crystallised from DIISOPROPYLETHER (5 ml) to give 5-Fluoro-N-(6-methoxy-pyridin-3-yl)-2-(3-methylsulfanyl-phenoxy)-nicotinamide (139 mg) as an off-white solid.

¹H NMR (400 MHz, CDCl₃): δ=9.47 (1H, s), 8.46-8.51 (1H, d), 8.18 (1H, s), 7.94-7.96 (1H, d), 7.83-7.88 (1H, dd), 7.20-7.26 (1H, t), 7.04-7.08 (1H, d), 6.93 (1H, s), 6.80-6.84 (1H, d), 6.60-6.65 (1H, d), 3.77 (3H, s), 2.36 (3H, s) ppm.

LRMS (electrospray): m/z [M+H]⁺ 386, [M+Na]⁺ 408, [2M+Na]⁺ 793, [M−H]⁺ 384.

Anal. Found C, 58.76; H, 4.14; N, 10.77. C₁₉H₁₆FN₃O₃S requires C, 59.21; H, 4.18; N, 10.90%.

### Example 58

### 5-Fluoro-N-(2-methoxy-pyridin-3-yl)-2-(3-methylsulfanyl-phenoxy)-nicotinamide

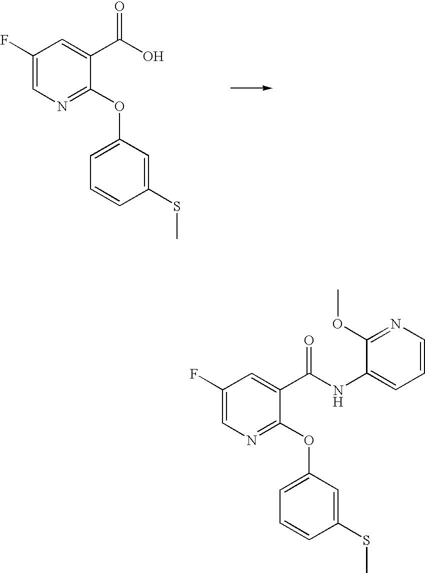

3-Amino-2-methoxypyridine (89 mg, 0.716 mmol) and triethylamine (200□l, 2.14 mmol) were dissolved in dimethylformamide (1 ml) under nitrogen at room temperature and a solution of 5-Fluoro-2-(3-methylsulfanyl-phenoxy)-nicotinic acid (200 mg, 0.716 mmol), 1-(3-dimethylaminopropyl)-3-ethylcarbodiimide hydrochloride (151 mg, 0.788 mmol) and 1-hydroxybenzotriazole (106 mg, 0.788 mmol) in dimethylformamide (5 ml) and the reaction was stirred at room temperature for 3 h. The reaction mixture was concentrated under reduced pressure, and the residue was purified by flash column chromatography on silica gel eluting with a solvent gradient of dichloromethane:methanol:concentrated aqueous ammonia (97.5:2.5:0.25, by volume) and the product was crystallised from DIISOPROPYLETHER (5 ml) to give 5-fluoro-N-(2-methoxy-pyridin-3-yl)-2-(3-methylsulfanyl-phenoxy)-nicotinamide (92 mg) as an off-white solid.

¹H NMR (400 MHz, CDCl₃): δ=10.57 (1H, s), 8.74-8.80 (1H, d), 8.40-8.44 (1H, dd), 8.12-8.14 (1H, d), 7.88-7.92 (1H, d), 7.37-7.42 (1H, t), 7.18-7.22 (1H, d), 7.17 (1H, s), 7.01-7.04 (1H, m), 6.94-6.98 (1H, m), 3.88 (3H, s), 2.53 (3H, s) ppm.

LRMS (electrospray): m/z [M+Na]⁺ 408, [2M+Na]⁺ 793, [M−H]⁺ 384.

Anal. Found C, 59.04; H, 4.16; N, 10.93. C₁₉H₁₆FN₃O₃S requires C, 59.21; H, 4.18; N, 10.90%.

### Example 59

### 5-Fluoro-N-(3-hydroxymethyl-pyridin-2-yl)-2-(3-methylsulfanyl-phenoxy)-nicotinamide

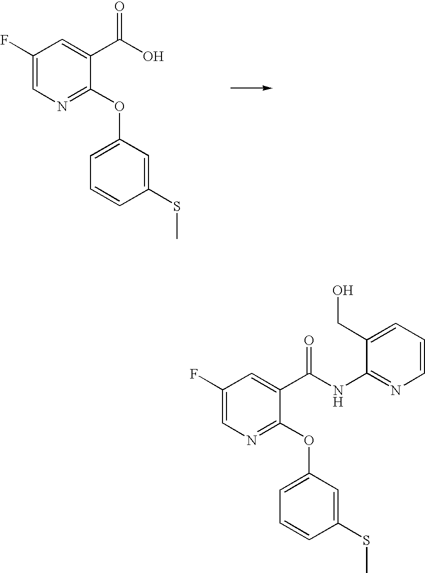

2-Hydroxymethyl-3-aminopyridine (89 mg, 0.716 mmol) and triethylamine (200□l, 2.14 mmol) were dissolved in dimethylformamide (1 ml) under nitrogen at room temperature and a solution of 5-Fluoro-2-(3-methylsulfanyl-phenoxy)-nicotinic acid (200 mg, 0.716 mmol), 1-(3-dimethylaminopropyl)-3-ethylcarbodiimide hydrochloride (151 mg, 0.788 mmol) and 1-hydroxybenzotriazole (106 mg, 0.788 mmol) in dimethylformamide (5 ml) and the reaction was stirred at room temperature for 18 h. The reaction mixture was concentrated under reduced pressure, and the residue was purified by flash column chromatography on silica gel eluting with a solvent gradient of dichloromethane:methanol:concentrated aqueous ammonia (97.5:2.5:0.25 changing to 95:5:0.5, by volume) and the product was crystallised from diisopropyl ether (5 ml) to give 5-Fluoro-N-(3-hydroxymethyl-pyridin-2-yl)-2-(3-methylsulfanyl-phenoxy)-nicotinamide (127 mg) as an off-white solid.

¹H NMR (400 MHz, CDCl₃): δ=8.14-8.16 (1H, d), 8.06-8,08 (1H, dd), 8.02-8.04 (1H, dd), 7.50-7.54 (1H, d), 7.28-7.32 (1H, t), 7.10-7.14 (1H, d), 6.97 (1H, s), 6.84-6.88 (1H, d), 6.46-6.50 (1H, d), 5.33 (2H, s), 4.89 (2H, brs), 2.45 (3H, s) ppm.

LRMS (electrospray): m/z [M+H]⁺ 386, [M+Na]⁺ 408, [2M+Na]⁺ 793, [M−H]⁺ 384.

Anal. Found C, 58.83; H, 4.16; N, 10.73. C₁₉H₁₆FN₃O₃S requires C, 59.21; H, 4.19; N, 10.90%.

### Example 60

### N-(3-Ethoxy-pyridin-2-yl)-5-fluoro-2-(3-methylsulfanyl-phenoxy)-nicotinamide

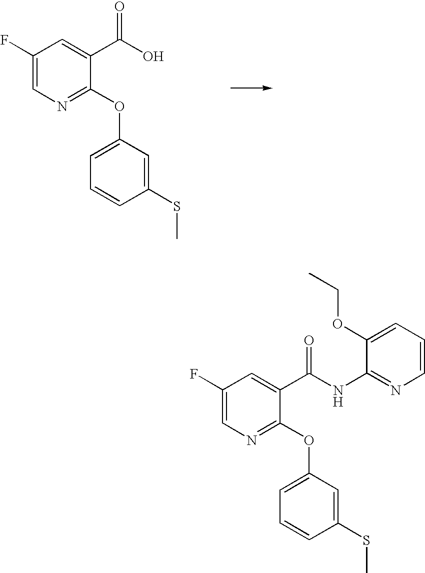

3-Ethoxy-2-aminopyridine (99 mg, 0.716 mmol) and triethylamine (200□l, 2.14 mmol) were dissolved in dimethylformamide (1 ml) under nitrogen at room temperature and a solution of 5-Fluoro-2-(3-methylsulfanyl-phenoxy)-nicotinic acid (200 mg, 0.716 mmol), 1-(3-dimethylaminopropyl)-3-ethylcarbodiimide hydrochloride (151 mg, 0.788 mmol) and 1-hydroxybenzotriazole (106 mg, 0.788 mmol) in dimethylformamide (5 ml) and the reaction was stirred at room temperature for 18 h. The reaction mixture was concentrated under reduced pressure, and the residue was purified by flash column chromatography on silica gel eluting with a solvent gradient of dichloromethane:methanol:concentrated aqueous ammonia (97.5:2.5:0.25, by volume) and the product was crystallised from diisopropylether (5 ml) to give N-(3-ethoxy-pyridin-2-yl)-5-fluoro-2-(3-methylsulfanyl-phenoxy)-nicotinamide (38 mg) as an off-white solid.

¹H NMR (400 MHz, CDCl₃): δ=10.59 (1H, brs), 8.46-8.52 (1H, dd), 8.14-8.18 (1H, d), 8.10-8.13 (1H, d), 7.35-7.40 (1H, t), 7.17-7.21 (1H, d), 7.08-7.15 (2H, m), 7.01-7.06 (1H, m), 6.94-6.98 (1H, m), 3.95-4.01 (2H, quart), 2.50 (3H, s), 1.03-1.11 (3H, t) ppm.

LRMS (electrospray): m/z [M+H]⁺ 400, [M+Na]⁺ 422, [2M+Na]⁺ 821, [M−H]⁺ 398.

Anal. Found C, 59.90; H, 4.50; N, 10.44. C₂₀H₁₈FN₃O₃S requires C, 60.14; H, 4.54; N, 10.52%.

### Example 61

### N-(2-Methyl-pyridin-3-yl)-5-fluoro-2-(3-methylsulfanyl-phenoxy)-nicotinamide

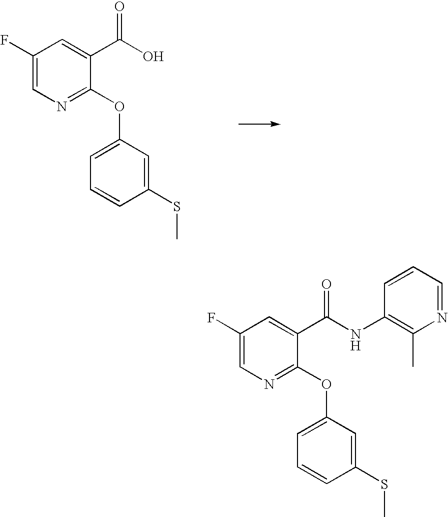

5-Fluoro-2-(3-methylsulfanyl-phenoxy)-nicotinic acid (150 mg, 0.54 mmol) was dissolved in dimethylformamide (5 ml) and triethylamine (225□l, 1.61 mmol) was added followed by 3-amino-2-methyl-pyridine (61 mg, 0.56 mmol), 1-(3-dimethylaminopropyl)-3-ethylcarbodiimide hydrochloride (113 mg, 0.59 mmol) and 1-hydroxybenzotriazole (80 mg, 0.59 mmol). The reaction was stirred under nitrogen at room temperature for 48 h and the solvent was removed under reduced pressure. The residue was partitioned between water (10 ml) and dichloromethane (20 ml) and the aqueous phase was extracted with dichloromethane (2×20 ml). The combined organic extracts were washed with brine (10 ml), dried over MgSO₄ and the solvent was removed under reduced pressure. The residual brown oil was purified by column chromatography on silica gel using pentane:ethylacetate (70:30) as eluent to give the title compound (96 mg) as an off-white solid.

¹H NMR (400 MHz, CDCl₃): δ=9.91 (1H, brs), 8.63-8.65 (1H, d), 8.45-8.47 (1H, dd), 8.30-8.31 (1H, d), 8.13-8.14 (1H, d), 7.38-7.42 (1H, t), 7.19-7.25 (2H, m), 7.07 (1H, s), 6.95-6.97 (1H, d), 2.52 (3H, s), 2.50 (3H, s) ppm.

LRMS (electrospray): mlz [M+H]⁺ 370 and [M+Na]⁺ 392.

Anal. Found C, 60.70; H, 4.41; N, 10.91. C₁₉H₁₆FN₃O₂S. 0.3 mol H₂O requires C, 60.88; H, 4.46; N, 11.21%.

### Example 62

### 5-Fluoro-N-(4-hydroxy-5-pyrimidinyl)-2-(3-methylsulfanyl-phenoxy)-nicotinamide

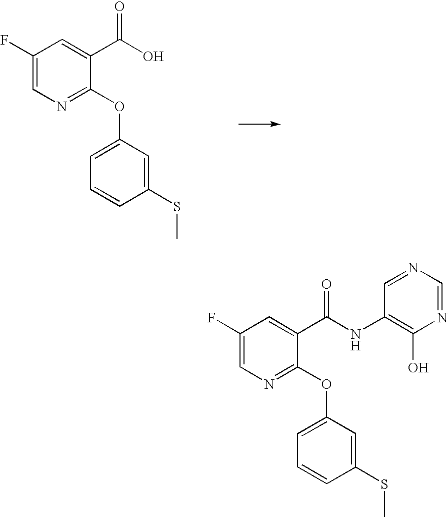

5-fluoro-2-(3-methylsulfanyl-phenoxy)-nicotinic acid (150 mg, 0.54 mmol) was dissolved in dimethylformamide (5 ml) and triethylamine (225□l, 1.61 mmol) was added followed by 3-amino-2-hydroxy-pyrimidine (85 mg, 0.56 mmol), 1-(3-dimethylaminopropyl)-3-ethylcarbodiimide hydrochloride (113 mg, 0.59 mmol) and 1-hydroxybenzotriazole (80 mg, 0.59 mmol). The reaction was stirred under nitrogen at room temperature for 48 h and the solvent was removed under reduced pressure. The residue was partitioned between water (10 ml) and dichloromethane (20 ml) and the aqueous phase was extracted with dichloromethane (2×20 ml). The combined organic extracts were washed with brine (10 ml), dried over MgSO₄ and the solvent was removed under reduced pressure. The residue was purified by column chromatography on silica gel using dichloromethane:methanol:ammonia (95:5:0.5) as eluent to give the title compound (80 mg) as an off-white solid.

¹H NMR (400 MHz, D₆-DMSO): δ=12.85 (1H, brs), 10.43 (1H, brs), 8.90 (1H, s), 8.34 (1H, d), 8.27-8.30 (1H, dd), 8.04 (1H, s), 7.35-7.39 (1H, t), 7.21 (1H, s), 7.14-7.16 (1H, d), 7.04-7.07 (1H, d) ppm. N.B. Suspect peak hidden under DMSO peak □□ 3.52 (3H, s) ppm.

LRMS (electrospray): m/z [M+Na]⁺ 395 and [M−H]⁻ 371.

Anal. Found C, 54.76; H, 3.53; N, 14.81. C₁₇H₁₃FN₄O₃S requires C, 54.83; H, 3.52; N, 15.05%.

In vitro Activity of the Nicotinamide Derivatives

The PDE4 inhibitory activity of the nicotinamide derivatives of the formula (1) is determined by the ability of compounds to inhibit the hydrolysis of cAMP to AMP by PDE4 (see also reference 1). Tritium labelled cAMP is incubated with PDE4. Following incubation, the radiolabelled AMP produced is able to bind yttrium silicate SPA beads. These SPA beads subsequently produce light that can be quantified by scintillation counting. The addition of a PDE4 inhibitor prevents the formation of AMP from cAMP and counts are diminished. The IC₅₀ of a PDE4 inhibitor can be defined as the concentration of a compound that leads to a 50% reduction in counts compared to the PDE4 only (no inhibitor) control wells.

The anti-inflammatory properties of the nicotinamide derivatives of the formula (1) are demonstrated by their ability to inhibit TNFα release from human peripheral blood mononuclear cells (see also reference 2). Venous blood is collected from healthy volunteers and the mononuclear cells purified by centrifugation through Histopaque (Ficoll) cushions. TNFα production from these cells is stimulated by addition of lipopolysaccharide. After 18 hours incubation in the presence of LPS, the cell supernatant is removed and the concentration of TNFα in the supernatant determined by ELISA. Addition of PDE4 inhibitors reduces the amount of TNFα produced. An IC₅₀ is determined which is equal to the concentration of compound that gives 50% inhibition of TNFα production as compared to the LPS stimulated control wells.

All the examples were tested in the assay described above and found to have an IC₅₀ (TNFα screen) of less than 300 nM. And for most of the tested compounds, they were found to have an IC₅₀ (TNFα screen) of even less than 100 nM.

It has been found that the compounds of Examples 1-47, and 56-69, all exhibit IC₅₀ values of 3 μM or less vs PDE4A, PDE4B and/or PDE4D.

References

1. Thompson J W, Teraski W L, Epstein P M, Strada S J., “Assay of nucleotidephosphodiesterase and resolution of multiple molecular forms of the isoenzyme”, Advances in cyclic nucleotides research, edited by Brooker G, Greengard P, Robinson G A. Raven Press, New York 1979, 10, p. 69-92.
2. Yoshimura T, Kurita C, Nagao T, Usami E, Nakao T, Watanabe S, Kobayashi J, Yamazaki F, Tanaka H, Nagai H., “Effects of cAMP-phosphodiesterase isozyme inhibitor on cytokine production by lipopolysaccharide-stimulated human peripheral blood mononuclear cells”, Gen. Pharmacol., 1997, 29(4), p. 63

1. A compound of formula (1) 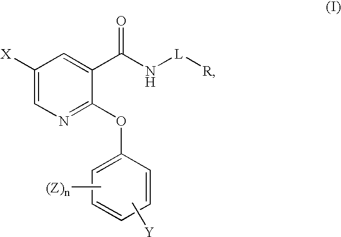
or a pharmaceutically acceptable salt or solvate thereof,
wherein:
X is hydrogen, methyl or halo;
Y and Z are taken separately and
Y is attached to the 3- or 4-position on the phenyl ring, and is S(O)ₚR¹, wherein R¹ is (C₁-C₄) alkyl optionally substituted by (C₃-C₆)cycloalkyl, and p is 0, 1 or 2;
n is 1 or 2;
Z is hydrogen, (C₁-C₃)alkyl, halo or (C₁-C₃)alkoxy, each Z being independently selected when n is 2;
or Y and (Z)ₙ, when attached to adjacent carbon atoms at the 3- or 4-position on the phenyl ring, are taken together with the carbon atoms to which they are attached to form a dihydrothienyl or dihydro-1,4-oxathiinyl ring;
L is a 5- or 6-membered heterocyclic ring containing one or two nitrogen ring atoms, which ring is optionally substituted independently by one to three hydroxy, hydroxy(C₁-C₃)alkyl, (C₁-C₃)alkoxy(C₁-C₃)alkyl, (C₁-C₃)alkyl, halo or (C₁-C₃)alkoxy;
R is H; (C₁-C₆)alkyl, hydroxy(C₁-C₆)alkyl, (C₁-C₃)alkoxy(C₁-C₆)alkyl, (C₁-C₃)alkyl)SO₂— or —COR²;
R² is
(C₁-C₆)alkyl optionally substituted by a 5- or 6-membered heterocyclic ring containing one to three hetero ring atoms independently selected from nitrogen, oxygen and sulfur, said heterocyclic ring being substituted independently by one to three three hydroxy, hydroxy(C₁-C₃)alkyl, (C₁-C₃)alkoxy(C₁-C₃)alkyl, (C₁-C₃)alkyl, halo or (C₁-C₃)alkoxy;
(C₁-C₃)alkoxy(C₁-C₆)alkyl;
hydroxy(C₁-C₆)alkyl;
NR³R⁴(C₁-C₆)alkyl;
(C₁-C₃)alkylCO₂(C₁-C₆)alkyl;
(C₁-C₃)alkylS(O)ₚ(C₁-C₆)alkyl;
(C₃-C₆)cycloalkyl;
a 5- or 6-membered heterocyclic ring containing one to three hetero ring atoms independently selected from nitrogen, oxygen and sulfur, which ring is optionally substituted independently by one to three (C₁-C₃)alkyl, hydroxy(C₁-C₃)alkyl, halo, oxo, hydroxy or (C₁-C₃)alkoxy; or
phenyl optionally substituted independently by one to three hydroxy, halo, (C₁-C₃)alkyl, (C₁-C₃)alkoxy, carboxy, CO₂(C₁-C₃)alkyl) or OC(═O)(C₁-C₃)alkyl; and
R³ and R⁴ are each independently H, (C₁-C₃)alkyl or C(O)(C₁-C₃)alkyl);
with the proviso that the compound is not
(i) 5-methyl-2-(3-methylsulphonyl)-N-(pyrazin-5-yl)nicotinamide;
(ii) 5-methyl-2-(3-ethylsulphonyl)-N-(pyrazin-5-yl)nicotinamide;
(iii) 2-(3-methylsulphonyl)-N-(pyrazin-5-yl)nicotinamide; or
(iv) 2-(3-ethylsulphonyl)-N-(pyrazin-5-yl)nicotinamide.

2. A compound of claim 1, or a pharmaceutically acceptable salt thereof, wherein X is F.

3. A compound of claim 1, or a pharmaceutically acceptable salt thereof, wherein p is 0.

4. A compound of claim 1, or a pharmaceutically acceptable salt thereof, wherein Y is attached to the 3-position on the phenyl ring.

5. A compound of claim 1, or a pharmaceutically acceptable salt thereof, wherein Y is S(O)ₚCH₃, S(O)ₚC₂H₅ or S(O)ₚCH₂-(cyclopropyl).

6. A compound of claim 1, or a pharmaceutically acceptable salt thereof, wherein Y is SCH₃, SC₂H₅ or SCH₂-(cyclopropyl).

7. A compound of claim 6, or a pharmaceutically acceptable salt thereof, wherein Y is SCH₃.

8. A compound of claim 6, or a pharmaceutically acceptable salt thereof, wherein Y is 3-SCH₃.

9. A compound of claim 1, or a pharmaceutically acceptable salt thereof, wherein when (Z)ₙ is not H or F and is attached to the 3-, 4-, and/or 5-position on the phenyl ring.

10. A compound of claim 1, or a pharmaceutically acceptable salt thereof, wherein (Z)ₙ is H.

11. A compound of claim 1, or a pharmaceutically acceptable salt thereof, wherein L is piperidinyl, pyrrolidinyl, pyrazinyl, pyridyl or pyrimidinyl, each of which is optionally substituted independently by one to three hydroxy, methoxy, hydroxymethyl, ethoxy or methyl.

12. A compound of claim 1, or a pharmaceutically acceptable salt thereof, wherein L is piperidin-1,3-ylenyl, piperidin-1,4-ylenyl, pyrazin-5,1-ylenyl, 3-hydroxypyridin-6,4-ylenyl, pyridin-4,2-ylenyl, pyridin-2,6-ylenyl, pyridin-4,6-ylenyl, pyridin-3,6-ylenyl, 3-methoxypyridin-6,4-ylenyl, 2-methoxypyridin-5,3-ylenyl, 2-methoxypyridin-3,5-ylenyl, 3-ethoxypyridin-2,6-ylenyl, 3-hydroxymethylpyridin-2,6-ylenyl, 2-methylpyridin-3,6-ylenyl, 3-methylpyridin-4,6-ylenyl, 4-hydroxypirimidin-2,5-ylenyl or 4-hydroxypyrimidin-5,2-ylenyl, where the first number of the linkage indicates the attachment to the NH of the nicotinamide moiety, and the second number of the linkage is attached to the R moiety, each L being optionally substituted independently by one to three hydroxy, hydroxy(C₁-C₃)alkyl, (C₁-C₃)alkoxy(C₁-C₃)alkyl, (C₁-C₃)alkyl, halo or (C₁-C₃)alkoxy.

13. A compound of claim 1 wherein L is piperidin-1,4-ylenyl, pyrazin-5,1-ylenyl, 3-hydroxypyridin-6,4-ylenyl, pyridin-4,2-ylenyl, pyridin-2,6-ylenyl, pyridin-4,6-ylenyl, pyridin-3,6-ylenyl, 2-methoxypyridin-3,5-ylenyl, 3-hydroxymethylpyridin-2,6-ylenyl or 3-methylpyridin-4,6-ylenyl, where the first number of the linkage indicates the attachment to the NH of the nicotinamide moiety, and the second number of the linkage is attached to the R moiety, each L being optionally substituted independently by one to three hydroxy, hydroxy(C₁-C₃)alkyl, (C₁-C₃)alkoxy(C₁-C₃)alkyl, (C₁-C₃)alkyl, halo or (C₁-C₃)alkoxy.

14. A compound of claim 13 wherein L is piperidin-1,4-ylenyl where the first number of the linkage indicates the attachment to the NH of the nicotinamide moiety, and the second number of the linkage is attached to the R moiety, said piperidin-1,4-ylenyl being optionally substituted independently by one to three hydroxy, hydroxy(C₁-C₃)alkyl, (C₁-C₃)alkoxy(C₁-C₃)alkyl, (C₁-C₃)alkyl, halo or (C₁-C₃)alkoxy.

15. A compound of claim 1 wherein R is attached to a nitrogen atom on the ring L.

16. A compound of claim 1 wherein R is H, (C₁-C₃)alkyl, SO₂(C₁-C₃)alkyl), or COR²;
R² is (C₁-C₃)alkoxy(C₁-C₃)alkyl; hydroxy(C₁-C₃)alkyl; NR³R⁴(C₁-C₃)alkyl; (C₁-C₃)alkyl optionally substituted by a 5- or 6-membered heterocyclic ring containing one to three hetero ring atoms independently selected from nitrogen, oxygen and sulfur; cyclopropyl; a 5- or 6-membered heterocyclic ring containing one to three hetero ring atoms independently selected from nitrogen, oxygen or sulfur, which ring is optionally substituted by (C₁-C₃)alkyl, hydroxy(C₁-C₃)alkyl, halo, oxo, hydroxy or (C₁-C₃)alkoxy; or phenyl optionally substituted by one to three substituents independently selected from hydroxy, halo, (C₁-C₃)alkyl, (C₁-C₃)alkoxy, carboxy, CO₂(C₁-C₃)alkyl) or OC(═O)(C₁-C₃)alkyl.

17. A compound of claim 1 wherein R is H, methyl, ethyl, methylcarbonyl, methylsulfonyl, ethylcarbonyl, pyridyl-CH₂CH₂-carbonyl, cyclopropylcarbonyl, hydroxymethylcarbonyl, (2-hydroxy-4-methylphenyl)carbonyl, (2-hydroxy-4-methoxyphenyl)carbonyl or (2-hydroxyphenyl)carbonyl.

18. A compound of claim 17 wherein R is H, methylcarbonyl or methylsulfonyl.

19. A compound of claim 18 wherein R is H or methylcarbonyl.

20. A process for preparing a compound of claim 1 comprising reacting a compound of formula (II), 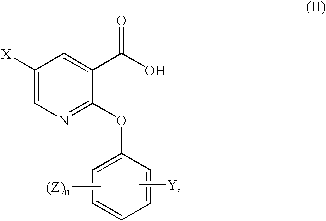
with a compound of formula NH₂—L—R, wherein X, (Z)ₙ, Y, L and R are defined as set forth in claim 1.

21. A process for preparing a compound of claim 1 comprising reacting a compound of formula (III), chloro-compound of formula (III), 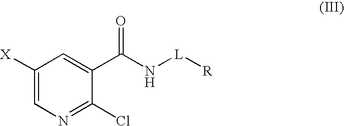
with a phenol of formula (IV), 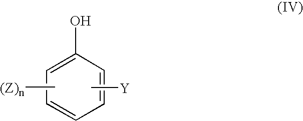
wherein X, (Z)ₙ, Y, L and R are defined as set forth in claim 1.

22. A pharmaceutical composition comprising a compound of claim 1 or a pharmaceutically acceptable salt or solvate thereof and a pharmaceutically acceptable excipient, diluent or carrier.

23. A method of treating a disease, disorder or condition in which PDE4 inhibition is beneficial in a mammal suffering from a disease, disorder or condition in which PDE4 inhibition is beneficial, said method comprising administering to said mammal in need of such treatment a therapeutically effective amount of a compound of claim 1, a pharmaceutically acceptable salt thereof or a pharmaceutical composition comprising a compound of claim 1 or a pharmaceutically acceptable salt thereof and a pharmaceutically acceptable carrier, diluent or excipient.

24. A method of claim 23 wherein the disease, disorder or condition is selected from:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy
pneumoconiosis of whatever type, etiology, or pathogenesis, in particular pneumoconiosis that is a member selected from the group consisting of aluminosis or bauxite workers' disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,
seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,
rheumatoid arthritis of whatever type, etiology, or pathogenesis, in particular rheumatoid arthritis that is a member selected from the group consisting of acute arthritis, acute gouty arthritis, chronic inflammatory arthritis, degenerative arthritis, infectious arthritis, Lyme arthritis, proliferative arthritis, psoriatic arthritis and vertebral arthritis,
gout, and fever and pain associated with inflammation,
an eosinophil-related disorder of whatever type, etiology, or pathogenesis, in particular an eosinophil-related disorder that is a member selected from the group consisting of eosinophilia, pulmonary infiltration eosinophilia, Loffler's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrotizing vasculitis,
atopic dermatitis, allergic dermatitis, contact dermatitis, or allergic or atopic eczema,
urticaria of whatever type, etiology, or pathogenesis, in particular urticaria that is a member selected from the group consisting of immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria and papular urticaria,
conjunctivitis of whatever type, etiology, or pathogenesis, in particular conjunctivitis that is a member selected from the group consisting of actinic conjunctivitis, acute catarrhal conjunctivitis, acute contagious conjunctivitis, allergic conjunctivitis, atopic conjunctivitis, chronic catarrhal conjunctivitis, purulent conjunctivitis and vernal conjunctivitis,
uveitis of whatever type, etiology, or pathogenesis, in particular uveitis that is a member selected from the group consisting of inflammation of all or part of the uvea, anterior uveitis, iritis, cyclitis, iridocyclitis, granulomatous uveitis, nongranulomatous uveitis, phacoantigenic uveitis, posterior uveitis, choroiditis; and chorioretinitis,
psoriasis;
multiple sclerosis of whatever type, etiology, or pathogenesis, in particular multiple sclerosis that is a member selected from the group consisting of primary progressive multiple sclerosis and relapsing remitting multiple sclerosis,
autoimmune/inflammatory diseases of whatever type, etiology, or pathogenesis, in particular an autoimmune/inflammatory disease that is a member selected from the group consisting of autoimmune hematological disorders, hemolytic anemia, aplastic anemia, pure red cell anemia, idiopathic thrombocytopenic purpura, systemic lupus erythematosus, polychondritis, scleroderma, Wegner's granulomatosis, dermatomyositis, chronic active hepatitis, myasthenia gravis, Stevens-Johnson syndrome, idiopathic sprue, autoimmune inflammatory bowel diseases, ulcerative colitis, endocrin opthamopathy, Grave's disease, sarcoidosis, alveolitis, chronic hypersensitivity pneumonitis, primary biliary cirrhosis, juvenile diabetes or diabetes mellitus type I, keratoconjunctivitis sicca, epidemic keratoconjunctivitis, diffuse interstitial pulmonary fibrosis or interstitial lung fibrosis, idiopathic pulmonary fibrosis, cystic fibrosis, glomerulonephritis with and without nephrotic syndrome, acute glomerulonephritis, idiopathic nephrotic syndrome, minimal change nephropathy, inflammatory/hyperproliferative skin diseases, benign familial pemphigus, pemphigus erythematosus, pemphigus foliaceus, and pemphigus vulgaris,
prevention of allogeneic graft rejection following organ transplantation,
inflammatory bowel disease (IBD) of whatever type, etiology, or pathogenesis, in particular inflammatory bowel disease that is a member selected from the group consisting of collagenous colitis, colitis polyposa, transmural colitis, ulcerative colitis and Crohn's disease (CD),
septic shock of whatever type, etiology, or pathogenesis, in particular septic shock that is a member selected from the group consisting of renal failure, acute renal failure, cachexia, malarial cachexia, hypophysial cachexia, uremic cachexia, cardiac cachexia, cachexia suprarenalis or Addison's disease, cancerous cachexia and cachexia as a consequence of infection by the human immunodeficiency virus (HIV),
liver injury,
pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension,
bone loss diseases, primary osteoporosis and secondary osteoporosis,
central nervous system disorders of whatever type, etiology, or pathogenesis, in particular a central nervous system disorder that is a member selected from the group consisting of depression, Alzheimers disease, Parkinson's disease, learning and memory impairment, tardive dyskinesia, drug dependence, arteriosclerotic dementia and dementias that accompany Huntington's chorea, Wilson's disease, paralysis agitans, and thalamic atrophies,
infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex,
yeast and fungus infections wherein said yeast and fungi are sensitive to upregulation by TNF-α or elicit TNF-α production in their host, e.g., fungal meningitis, particularly when administered in conjunction with other drugs of choice for the treatment of systemic yeast and fungus infections, including but are not limited to, polymixins, e.g. Polymycin B, imidazoles, e.g. clotrimazole, econazole, miconazole, and ketoconazole, triazoles, e.g. fluconazole and itranazole as well as amphotericins, e.g. Amphotericin B and liposomal Amphotericin B,
ischemia-reperfusion injury, ischemic heart disease, autoimmune diabetes, retinal autoimmunity, chronic lymphocytic leukemia, HIV infections, lupus erythematosus, kidney and ureter disease, urogenital and gastrointestinal disorders and prostate diseases,
reduction of scar formation in the human or animal body, such as scar formation in the healing of acute wounds, and psoriasis, other dermatological and cosmetic uses, including antiphlogistic, skin-softening, skin elasticity and moisture-increasing activities.

25. A method of claim 24 wherein the disease, disorder or condition is chronic obstructive pulmonary disease, asthma or chronic bronchitis.

26. A combination of a compound of claim 1 or a pharmaceutically acceptable salt or solvate thereof with other therapeutic agents selected from:
(a) 5-Lipoxygenase (5-LO) inhibitors or 5-lipoxygenase activating protein (FLAP) antagonists,
(b) Leukotriene antagonists (LTRAs) including antagonists of LTB4, LTC4, LTD4, and LTE4,
(c) Histaminic receptor antagonists including H1, H3 and H4 antagonists,
(d) α1- and α2-adrenoceptor agonist vasoconstrictor sympathomimetic agents for decongestant use,
(e) Muscarinic M3 receptor antagonists or anticholinergic agents,
(f) β2-adrenoceptor agonists,
(g) Theophylline,
(h) Sodium cromoglycate,
(i) COX-1 inhibitors (NSAIDs) and COX-2 selective inhibitors,
(j) Oral or inhaled Glucocorticosteroids,
(k) Monoclonal antibodies active against endogenous inflammatory entities,
(l) Anti-tumor necrosis factor (anti-TNF-α) agents,
(m) Adhesion molecule inhibitors including VLA-4 antagonists,
(n) Kinin-B1- and B2-receptor antagonists,
(o) Immunosuppressive agents,
(p) Inhibitors of matrix metalloproteases (MMPs),
(q) Tachykinin NK1, NK2 and NK3 receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFkb pathway, e.g. IKK inhibitors,
(w) Agents that can be classed as mucolytics or anti-tussive,
(x) antibiotics, and
(y) p38 MAP kinase inhibitors.
